(19) United States
(12) Patent Application Publication
(10) Pub. No.: US 2005/0096278 A1
(43) Pub. Date: May 5, 2005

(54) Antibacterial agents

(76) Inventors: Edmund Lee Ellsworth, Pinckney, MI (US); Chris Limberakis, Saline, MI (US); Clarke Taylor, Arbor, MI (US)

Correspondence Address: WARNER-LAMBERT COMPANY, 2800 PLYMOUTH RD, ANN ARBOR, MI, 48105, US

(21) Appl. No.: 10/936,224

(22) Filed: Sep. 8, 2004

Related U.S. Application Data
(60) Provisional application No. 60/502,328, filed on Sep. 12, 2003.

(51) Int. Cl. C07F 5/02; A61K 31/69; A61K 38/04; A61K 31/4745; C07K 5/04; C07D 471/02

(52) U.S. Cl. 514/19; 514/64; 514/300; 546/123; 546/13

(57) ABSTRACT

Compounds of formula I, II, III, IV, IV, and VI and methods for their preparation are disclosed. Further disclosed are methods of making biologically active compounds of formula I as well as pharmaceutically acceptable compositions comprising compounds of formula I. Compounds of formula I as disclosed herein can be used in a variety of applications including use as antibacterial agents.

### FIELD OF THE INVENTION

The invention relates to compounds which exhibit antibacterial activity, methods for their preparation, as well as pharmaceutically acceptable compositions comprising such compounds.

### BACKGROUND OF THE INVENTION

Antibacterial resistance is a global clinical and public health problem that has emerged with alarming rapidity in recent years and undoubtedly will increase in the near future. Resistance is a problem in the community as well as in health care settings, where transmission of bacteria is greatly amplified. Because multiple drug resistance is a growing problem, physicians are now confronted with infections for which there is no effective therapy. The morbidity, mortality, and financial costs of such infections pose an increasing burden for health care systems worldwide. Strategies to address these issues emphasize enhanced surveillance of drug resistance, increased monitoring and improved usage of antimicrobial drugs, professional and public education, development of new drugs, and assessment of alternative therapeutic modalities.

As a result, alternative and improved agents are needed for the treatment of bacterial infections, particularly for the treatment of infections caused by resistant strains of bacteria, e.g. penicillin-resistant, methicillin-resistant, ciprofloxacin-resistant, and/or vancomycin-resistant strains.

### SUMMARY OF THE INVENTION

These and other needs are met by the present invention, which is directed to a compound of formula I 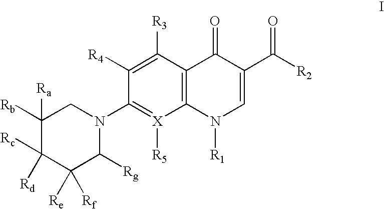
or a pharmaceutically acceptable salt thereof, wherein:
X is N or C, provided that when X is N, R₅ is absent at that position;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is OH,
   OBF₂,
   O(C₁-C₆)alkyl,
   O(C₃-C₆)cycloalkyl, 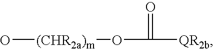
   wherein m is an integer of from 1 to 10, Q is O or is absent, and R₂ₐ is H or (C₁-C₆)alkyl and R_{2b} is (C₁-C₆)alkyl, aryl, or heteroaryl,
   O—(CHR₂ₐ)ₙ—Y, wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
   NR_{2d}, wherein R_{2d} is as defined above, 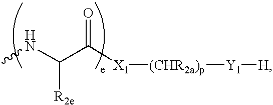
   wherein “ 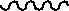
   ” indicates the point of attachment, 2a is as defined above, R₂ₑ is H or (C₁-C₆)alkyl, e is an integer of from 1 to 10, p is an integer of from 2 to 10, and X₁ and Y₁ are each independently NH or O;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
R₁ and R₅, together with the carbons to which they are attached, form a substituted or unsubstituted 6-membered ring containing an additional heteroatom selected from O, S, NH, or N(C₁-C₆)alkyl;
at least one of Rₐ, R_{b}, R_{c}, R_{d}, R_{d}, R_{f}, and R_{g} is
   OH,
   OPO(OH)₂,
   OPO(O(C₁-C₆)alkyl)₂, 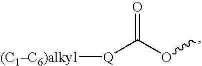
   wherein “ 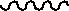
   ” indicates the point of attachment and Q is O or is absent 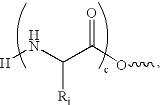
   wherein “ 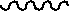
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)hydroxyalkyl
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 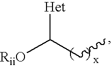
   wherein “ 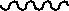
   ” indicates the point of attachment, het is a 5- or 6-membered heterocyclo or heteroaryl group, and x is an integer of from 0 to 10; 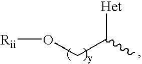
   wherein “ 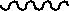
   ” indicates the point of attachment, het is as defined above, and y is an integer of from 1 to 10^{;}
      wherein Rᵢᵢ is H,
         (C₁-C₆)alkyl,
         PO(OH)₂,
         PO(O(C₁-C₆)alkyl)₂, 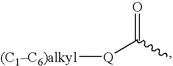
         as defined above, or 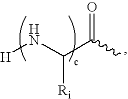
         as defined above;
and the others of Rₐ, R_{b}, R_{c}, R_{d}, Rₑ, R_{f}, and R_{g} are each independently H,
   (C₁-C₆)alkyl,
   OH,
   OPO(OH)₂,
   OPO(O(C₁-C₆)alkyl)₂, 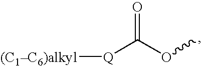
   wherein “ 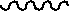
   ” indicates the point of attachment and Q is O or is absent 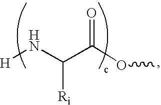
   wherein “ 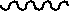
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 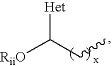
   wherein “ 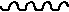
   ” indicates the point of attachment, het is a 5- or 6-membered heterocyclo or heteroaryl group, and x is an integer of from 0 to 10; 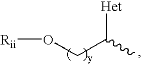
   wherein “ 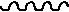
   ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, 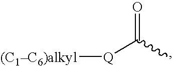
         as defined above, or 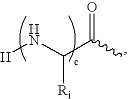
         as defined above; or
   R_{c} and Rₑ, together with the carbons to which they are attached, form a substituted or unsubstituted 4, 5, or 6-membered ring containing 0, 1, 2, or 3 heteroatoms selected from P, NH, N(C₁-C₆)alkyl, S, or O; or
   R_{c} and R_{d} or Rₑ and R_{f}, independently, together with the carbon to which they are attached, form a substituted or unsubstituted 3, 4, 5, or 6-membered ring containing 0, 1, 2, or 3 heteroatoms selected from NH, N(C₁-C₆)alkyl S, or O; and
   provided that when R₅ is OMe, and R_{c} is OH, R_{d} is not H or Me.

What is also provided is a compound of formula II: 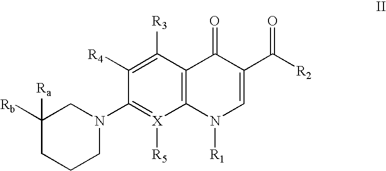
or a pharmaceutically acceptable salt thereof, wherein:
X, and R₁-R₅ are as defined for compounds of formula I and
Rₐ is OH,
   OPO(OH)₂,
   OPO(O(C₁-C₆)alkyl)₂, 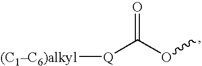
   wherein “ 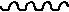
   ” indicates the point of attachment and Q is O or is absent 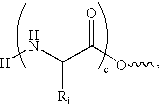
   wherein “ 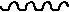
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)hydroxyalkyl
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 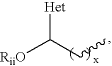
   wherein “ 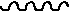
   ” indicates the point of attachment, het is a 5- or 6-membered heterocyclo or heteroaryl group, and x is an integer of from 0 to 10; 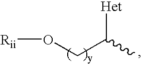
   wherein “ 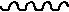
   ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, 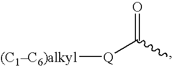
         as defined above, or 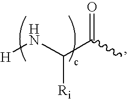
         as defined above;
and R_{b} is
   H,
   (C₁-C₆)alkyl, 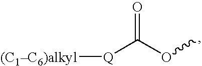
   wherein “ 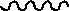
   ” indicates the point of attachment and Q is O or is absent 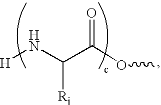
   wherein “ 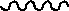
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)hydroxyalkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 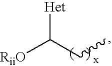
   wherein “ 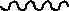
   ” indicates the point of attachment, het is a 5- or 6-membered heterocyclo or heteroaryl group, and x is an integer of from 0 to 10; 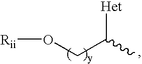
   wherein “ 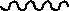
   ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, 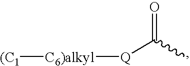
         as defined above, or 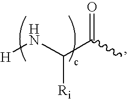
         as defined above.

What is also provided is a compound which is 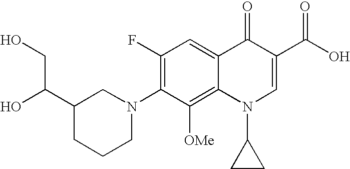
1-Cyclopropyl-7-[3-(1,2-dihydroxy-ethyl)-piperidin-1-yl]-6-fluoro-8-methoxy-4-oxo-1,4-dihydro-quinoline-3-carboxylic acid; 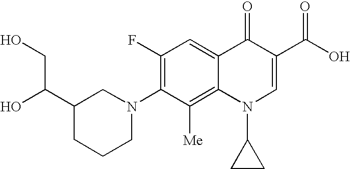
1-Cyclopropyl-7-[3-(1,2-dihydroxy-ethyl)-piperidin-1-yl]-6-fluoro-8-methyl-4-oxo-1,4-dihydro-quinoline-3-carboxylic acid; 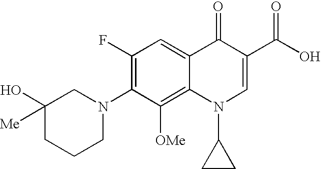
1-Cyclopropyl-6-fluoro-7-(3-hydroxy-3-methyl-piperidin-1-yl)-8-methoxy-4-oxo-1,4-dihydro-quinoline-3-carboxylic acid; 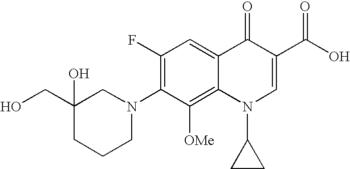
1-Cyclopropyl-6-fluoro-7-(3-hydroxy-3-hydroxymethyl-piperidin-1-yl)-8-methoxy-4-oxo-1,4-dihydro-quinoline-3-carboxylic acid; 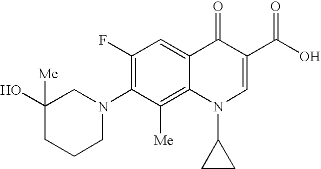
1-Cyclopropyl-6-fluoro-7-(3-hydroxy-3-methyl-piperidin-1-yl)-8-methyl-4-oxo-1,4-dihydro-quinoline-3-carboxylic acid;
1-Cyclopropyl-7-[3-(1,2-dihydroxy-ethyl)-piperidin-1-yl]-8-methoxy-4-oxo-1,4-dihydro-quinoline-3-carboxylic acid;
1-Cyclopropyl-7-[3-(1,2-dihydroxy-ethyl)-piperidin-1-yl]-8-methyl-4-oxo-1,4-dihydro-quinoline-3-carboxylic acid;
1-Cyclopropyl-7-(3-hydroxy-3-methyl-piperidin-1-yl)-8-methoxy-4-oxo-1,4-dihydro-quinoline-3-carboxylic acid;
1-Cyclopropyl-7-(3-hydroxy-3-hydroxymethyl-piperidin-1-yl)-8-methoxy-4-oxo-1,4-dihydro-quinoline-3-carboxylic acid; or
1-Cyclopropyl-7-(3-hydroxy-3-methyl-piperidin-1-yl)-8-methyl-4-oxo-1,4-dihydro-quinoline-3-carboxylic acid.

What is also provided is a compound of formula III: 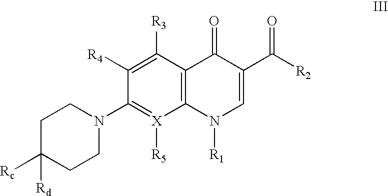
or a pharmaceutically acceptable salt thereof, wherein:
X, and R₁-R₅ are as defined for compounds of formula I and
R_{c} is OH,
   OPO(OH)₂,
   OPO(O(C₁-C₆)alkyl)₂, 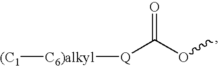
   wherein “ 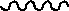
   ” indicates the point of attachment and Q is O or is absent 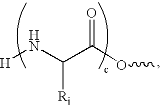
   wherein “ 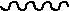
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)hydroxyalkyl
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 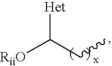
   wherein “ 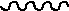
   ” indicates the point of attachment, het is a 5- or 6-membered heterocyclo or heteroaryl group, and x is an integer of from 0 to 10; 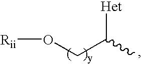
   wherein “ 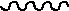
   ” indicates the point of attachment, het is as defined above, and y is an integer of from 1 to 10;
      wherein Rᵢᵢ is H,
         (C₁-C₆)alkyl,
         P(OH)₂,
         PO(O(C₁-C₆)alkyl)₂, 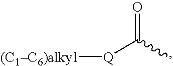
         as defined above, or 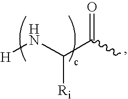
         as defined above;
and R_{d} is
   H,
   (C₁-C₆)alkyl, 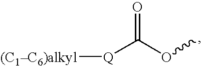
   wherein “ 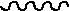
   ” indicates the point of attachment and Q is O or is absent 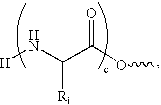
   wherein “ 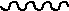
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)hydroxyalkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 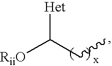
   wherein “ 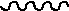
   ” indicates the point of attachment, het is a 5- or 6-membered heterocyclo or heteroaryl group, and x is an integer of from 0 to 10; 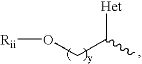
   wherein “ 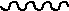
   ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, 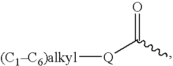
         as defined above, or 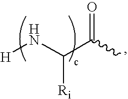
            as defined above.

What is also provided is a compound which is 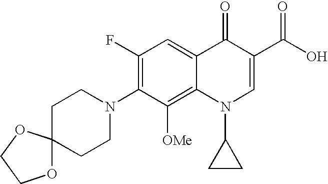
1-Cyclopropyl-7-(1,4-dioxa-8-aza-spiro[4.5]dec-8-yl)-6-fluoro-8-methoxy-4-oxo-1,4-dihydro-quinoline-3-carboxylic acid; 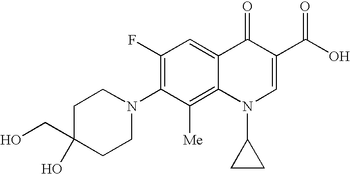
1-Cyclopropyl-6-fluoro-7-(4-hydroxy-4-hydroxymethyl-piperidin-1-yl)-8-methyl-4-oxo-1,4-dihydro-quinoline-3-carboxylic acid; 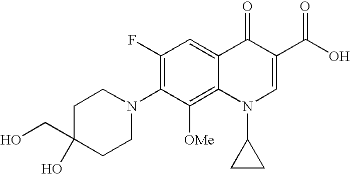
1-Cyclopropyl-6-fluoro-7-(4-hydroxy-4-hydroxymethyl-piperidin-1-yl)-8-methoxy-4-oxo-1,4-dihydro-quinoline-3-carboxylic acid; 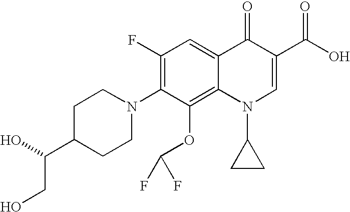
1-Cyclopropyl-8-difluoromethoxy-7-[4-(1,2-dihydroxy-ethyl)-piperidin-1-yl]-6-fluoro-4-oxo-1,4-dihydro-quinoline-3-carboxylic acid; 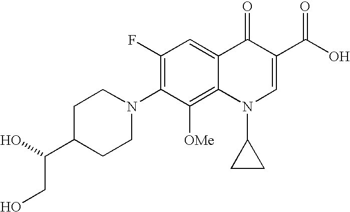
1-Cyclopropyl-8-methoxy-7-[4-(1,2-dihydroxy-ethyl)-piperidin-1-yl]-6-fluoro-4-oxo-1,4-dihydro-quinoline-3-carboxylic acid; 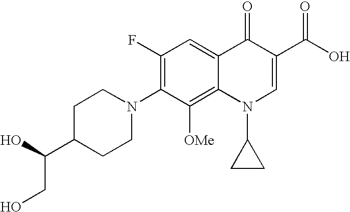
1-Cyclopropyl-8-methyl-7-[4-(1,2-dihydroxy-ethyl)-piperidin-1-yl]-6-fluoro-4-oxo-1,4-dihydro-quinoline-3-carboxylic acid; 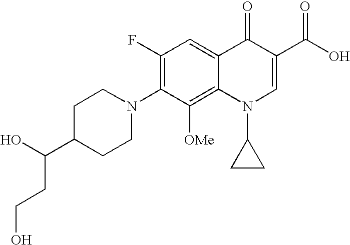
1-Cyclopropyl-7-[4-(1,3-dihydroxy-propyl)-piperidin-1-yl]-6-fluoro-8-methoxy-4-oxo-1,4-dihydro-quinoline-3-carboxylic acid; 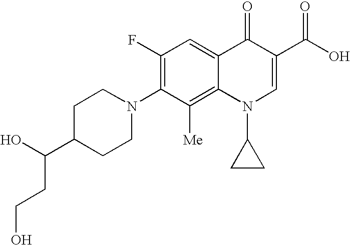
1-Cyclopropyl-7-[4-(1,3-dihydroxy-propyl)-piperidin-1-yl]-6-fluoro-8-methyl-4-oxo-1,4-dihydro-quinoline-3-carboxylic acid; 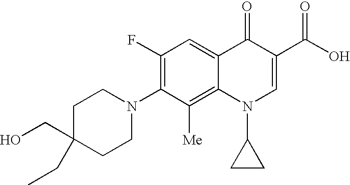
7-(4,4-Bis-hydroxymethyl-piperidin-1-yl)-1-cyclopropyl-6-fluoro-8-methyl-4-oxo-1,4-dihydro-quinoline-3-carboxylic acid; 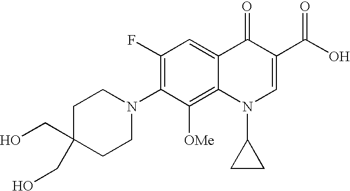
7-(4,4-Bis-hydroxymethyl-piperidin-1-yl)-1-cyclopropyl-6-fluoro-8-methoxy-4-oxo-1,4-dihydro-quinoline-3-carboxylic acid;
1-Cyclopropyl-7-(1,4-dioxa-8-aza-spiro[4.5]dec-8-yl)-8-methoxy-4-oxo-1,4-dihydro-quinoline-3-carboxylic acid; 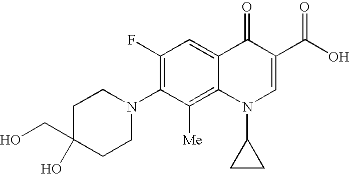
1-Cyclopropyl-7-(4-hydroxy-4-hydroxymethyl-piperidin-1-yl)-8-methyl-4-oxo-1,4-dihydro-quinoline-3-carboxylic acid;
1-Cyclopropyl-7-(4-hydroxy-4-hydroxymethyl-piperidin-1-yl)-8-methoxy-4-oxo-1,4-dihydro-quinoline-3-carboxylic acid;
1-Cyclopropyl-8-difluoromethoxy-7-[4-(1,2-dihydroxy-ethyl)-piperidin-1-yl]-4-oxo-1,4-dihydro-quinoline-3-carboxylic acid;
1-Cyclopropyl-8-methoxy-7-[4-(1,2-dihydroxy-ethyl)-piperidin-1-yl]-4-oxo-1,4-dihydro-quinoline-3-carboxylic acid;
1-Cyclopropyl-8-methyl-7-[4-(1,2-dihydroxy-ethyl)-piperidin-1-yl]-4-oxo-1,4-dihydro-quinoline-3-carboxylic acid;
1-Cyclopropyl-7-[4-(1,3-dihydroxy-propyl)-piperidin-1-yl]-8-methoxy-4-oxo-1,4-dihydro-quinoline-3-carboxylic acid;
1-Cyclopropyl-7-[4-(1,3-dihydroxy-propyl)-piperidin-1-yl]-8-methyl-4-oxo-1,4-dihydro-quinoline-3-carboxylic acid;
7-(4,4-Bis-hydroxymethyl-piperidin-1-yl)-1-cyclopropyl-8-methyl-4-oxo-1,4-dihydro-quinoline-3-carboxylic acid; or
7-(4,4-Bis-hydroxymethyl-piperidin-1-yl)-1-cyclopropyl-8-methoxy-4-oxo-1,4-dihydro-quinoline-3-carboxylic acid.

What is also provided is a compound of formula IV 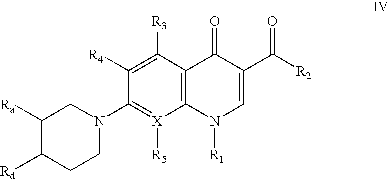
or a pharmaceutically acceptable salt thereof, wherein:
X, and R₁-R₅ are as defined for compounds of formula I and
Rₐ is OH,
   OPO(OH)₂,
   OPO(O(C₁-C₆)alkyl)₂, 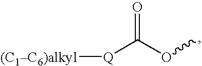
   wherein “ 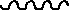
   ” indicates the point of attachment and Q is O or is absent 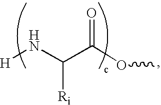
   wherein “ 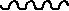
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)hydroxyalkyl
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 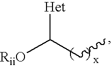
   wherein “ 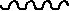
   ” indicates the point of attachment, het is a 5- or 6-membered heterocyclo or heteroaryl group, and x is an integer of from 0 to 10; 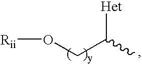
   wherein “ 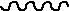
   ” indicates the point of attachment, het is as defined above, and y is an integer of from 1 to 10; wherein Rᵢᵢ is H,
      (C₁-C₆)alkyl,
      PO(OH)₂,
      PO(O(C₁-C₆)alkyl)₂, 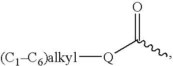
   as defined above, or 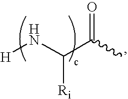
   as defined above;
and R_{d} is
   (C₁-C₆)alkyl, 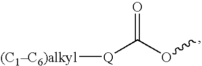
   wherein “ 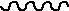
   ” indicates the point of attachment and Q is O or is absent 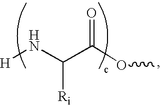
   wherein “ 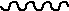
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)hydroxyalkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 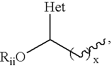
   wherein “ 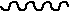
   ” indicates the point of attachment, het is a 5- or 6-membered heterocyclo or heteroaryl group, and x is an integer of from 0 to 10; 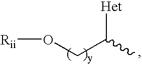
   wherein “ 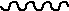
   ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, 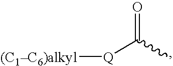
      as defined above, or 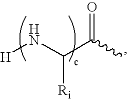
         as defined above.

What is also provided is a compound which is 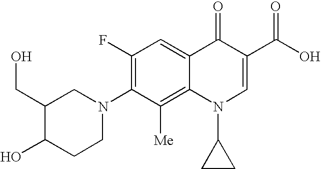
1-Cyclopropyl-6-fluoro-7-(4-hydroxy-3-hydroxymethyl-piperidin-1-yl)-8-methyl-4-oxo-1,4-dihydro-quinoline-3-carboxylic acid; 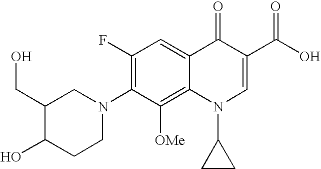
1-Cyclopropyl-6-fluoro-7-(4-hydroxy-3-hydroxymethyl-piperidin-1-yl)-8-methoxy-4-oxo-1,4-dihydro-quinoline-3-carboxylic acid; 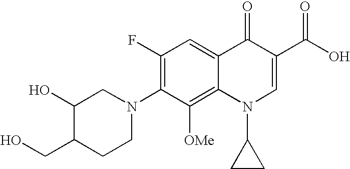
1-Cyclopropyl-6-fluoro-7-(3-hydroxy-4-hydroxymethyl-piperidin-1-yl)-8-methoxy-4-oxo-1,4-dihydro-quinoline-3-carboxylic acid; 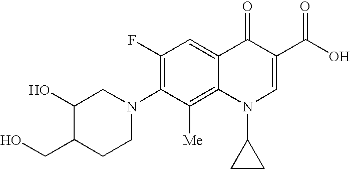
1-Cyclopropyl-6-fluoro-7-(3-hydroxy-4-hydroxymethyl-piperidin-1-yl)-8-methyl-4-oxo-1,4-dihydro-quinoline-3-carboxylic acid; 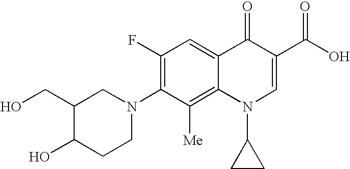
1-Cyclopropyl-6-fluoro-7-(4-hydroxy-3-hydroxymethyl-piperidin-1-yl)-8-methyl-4-oxo-1,4-dihydro-quinoline-3-carboxylic acid; 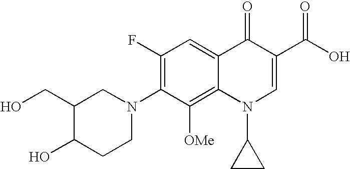
1-Cyclopropyl-6-fluoro-7-(4-hydroxy-3-hydroxymethyl-piperidin-1-yl)-8-methoxy-4-oxo-1,4-dihydro-quinoline-3-carboxylic acid;
1-Cyclopropyl-7-(4-hydroxy-3-hydroxymethyl-piperidin-1-yl)-8-methyl-4-oxo-1,4-dihydro-quinoline-3-carboxylic acid;
1-Cyclopropyl-7-(4-hydroxy-3-hydroxymethyl-piperidin-1-yl)-8-methoxy-4-oxo-1,4-dihydro-quinoline-3-carboxylic acid;
1-Cyclopropyl-7-(3-hydroxy-4-hydroxymethyl-piperidin-1-yl)-8-methoxy-4-oxo-1,4-dihydro-quinoline-3-carboxylic acid;
1-Cyclopropyl-7-(3-hydroxy-4-hydroxymethyl-piperidin-1-yl)-8-methyl-4-oxo-1,4-dihydro-quinoline-3-carboxylic acid;
1-Cyclopropyl-7-(4-hydroxy-3-hydroxymethyl-piperidin-1-yl)-8-methyl-4-oxo-1,4-dihydro-quinoline-3-carboxylic acid; or
1-Cyclopropyl-7-(4-hydroxy-3-hydroxymethyl-piperidin-1-yl)-8-methoxy-4-oxo-1,4-dihydro-quinoline-3-carboxylic acid.

What is also provided is a compound or formula V 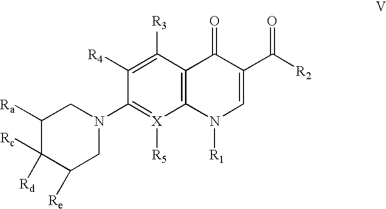
or a pharmaceutically acceptable salt thereof, wherein:
X, and R₁-R₅ are as defined for compounds of formula I and
Rₐ and Rₑ are each independently (C₁-C₆)alkyl or hydroxy(C₁-C₆)alkyl;
R_{d} is H or OH;
R_{c} is hydroxy(C₁-C₆)alkyl;
provided that when R₅ is OMe, and R_{c} is OH, R_{d} is not H or Me.

What is also provided is a compound which is 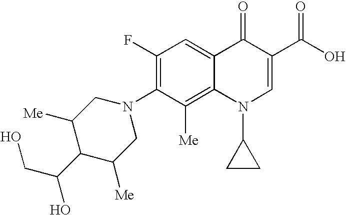
1-Cyclopropyl-7-[4-(1,2-dihydroxy-ethyl)-3,5-dimethyl-piperidin-1-yl]-6-fluoro-8-methyl-4-oxo-1,4-dihydro-quinoline-3-carboxylic acid, 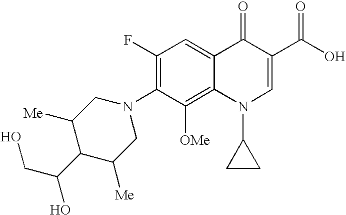
1-Cyclopropyl-7-[4-(1,2-dihydroxy-ethyl)-3,5-dimethyl-piperidin-1-yl]-6-fluoro-8-methoxy-4-oxo-1,4-dihydro-quinoline-3-carboxylic acid; 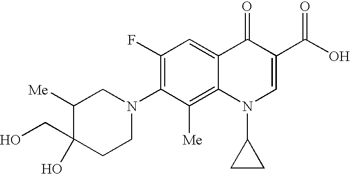
1-Cyclopropyl-6-fluoro-7-(4-hydroxy-4-hydroxymethyl-3-methyl-piperidin-1-yl)-8-methyl-4-oxo-1,4-dihydro-quinoline-3-carboxylic acid; 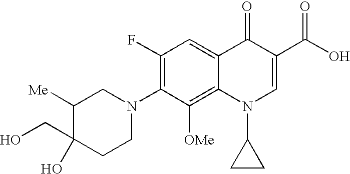
1-Cyclopropyl-6-fluoro-7-(4-hydroxy-4-hydroxymethyl-3-methyl-piperidin-1-yl)-8-methoxy-4-oxo-1,4-dihydro-quinoline-3-carboxylic acid; 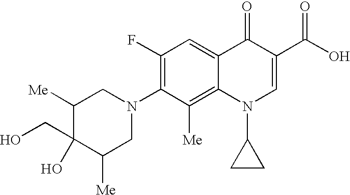
1-Cyclopropyl-6-fluoro-7-(4-hydroxy-4-hydroxymethyl-3,5-dimethyl-piperidin-1-yl)-8-methyl-4-oxo-1,4-dihydro-quinoline-3-carboxylic acid; 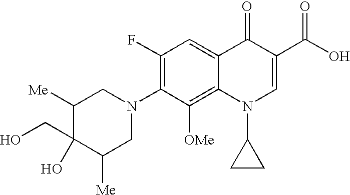
1-Cyclopropyl-6-fluoro-7-(4-hydroxy-4-hydroxymethyl-3,5-dimethyl-piperidin-1-yl)-8-methoxy-4-oxo-1,4-dihydro-quinoline-3-carboxylic acid; 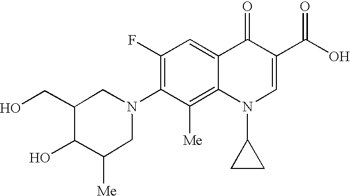
1-Cyclopropyl-6-fluoro-7-(4-hydroxy-3-hydroxymethyl-5-methyl-piperidin-1-yl)-8-methyl-4-oxo-1,4-dihydro-quinoline-3-carboxylic acid; 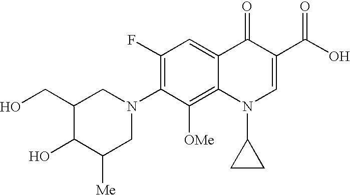
1-Cyclopropyl-6-fluoro-7-(4-hydroxy-3-hydroxymethyl-5-methyl-piperidin-1-yl)-8-methoxy-4-oxo-1,4-dihydro-quinoline-3-carboxylic acid; 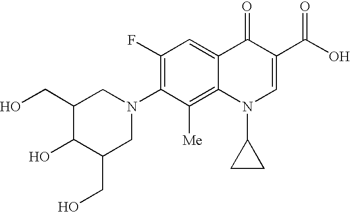
1-Cyclopropyl-6-fluoro-7-(4-hydroxy-3,5-bis-hydroxymethyl-piperidin-1-yl)-8-methyl-4-oxo-1,4-dihydro-quinoline-3-carboxylic acid; 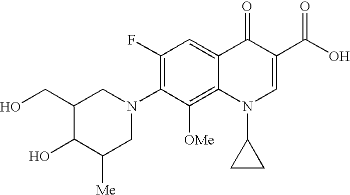
1-Cyclopropyl-6-fluoro-7-(4-hydroxy-3,5-bis-hydroxymethyl-piperidin-1-yl)-8-methoxy-4-oxo-1,4-dihydro-quinoline-3-carboxylic acid;
1-Cyclopropyl-7-[4-(1,2-dihydroxy-ethyl)-3,5-dimethyl-piperidin-1-yl]-8-methyl-4-oxo-1,4-dihydro-quinoline-3-carboxylic acid,
1-Cyclopropyl-7-[4-(1,2-dihydroxy-ethyl)-3,5-dimethyl-piperidin-1-yl]-8-methoxy-4-oxo-1,4-dihydro-quinoline-3-carboxylic acid;
1-Cyclopropyl-7-(4-hydroxy-4-hydroxymethyl-3-methyl-piperidin-1-yl)-8-methyl-4-oxo-1,4-dihydro-quinoline-3-carboxylic acid;
1-Cyclopropyl-7-(4-hydroxy-4-hydroxymethyl-3-methyl-piperidin-1-yl)-8-methoxy-4-oxo-1,4-dihydro-quinoline-3-carboxylic acid; 1-Cyclopropyl-7-(4-hydroxy-4-hydroxymethyl-3,5-dimethyl-piperidin-1-yl)-8-methyl-4-oxo-1,4-dihydro-quinoline-3-carboxylic acid;
1-Cyclopropyl-7-(4-hydroxy-4-hydroxymethyl-3,5-dimethyl-piperidin-1-yl)-8-methoxy-4-oxo-1,4-dihydro-quinoline-3-carboxylic acid;
1-Cyclopropyl-7-(4-hydroxy-3-hydroxymethyl-5-methyl-piperidin-1-yl)-8-methyl-4-oxo-1,4-dihydro-quinoline-3-carboxylic acid;
1-Cyclopropyl-7-(4-hydroxy-3-hydroxymethyl-5-methyl-piperidin-1-yl)-8-methoxy-4-oxo-1,4-dihydro-quinoline-3-carboxylic acid;
1-Cyclopropyl-7-(4-hydroxy-3,5-bis-hydroxymethyl-piperidin-1-yl)-8-methyl-4-oxo-1,4-dihydro-quinoline-3-carboxylic acid; or
1-Cyclopropyl-7-(4-hydroxy-3,5-bis-hydroxymethyl-piperidin-1-yl)-8-methoxy-4-oxo-1,4-dihydro-quinoline-3-carboxylic acid.

What is also provided is a compound of formula VI 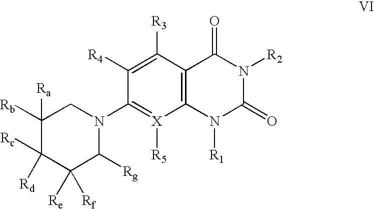
or a pharmaceutically acceptable salt thereof, wherein:
X is N or C, provided that when X is N, R₅ is absent at that position;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is H,
   NH₂, 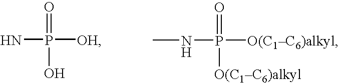
   NH(C₁-C₆)alkyl,
   NH(C₃-C₆)cycloalkyl,
   NH-heteroaryl,
   NHSO₂—(C₁-C₆)alkyl,
   NHSO₂-aryl,
   NHSO₂-heteroaryl, 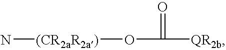
   wherein, Q is O or is absent, and R₂ₐ and R_{2a′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 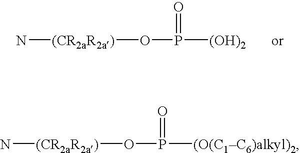
   wherein R₂ₐ and R_{2a′} are as defined above, 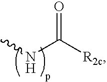
   wherein “ 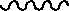
   ” indicates the point of attachment, p is 0 or 1, and
      R_{2c} is H,
         (C₁-C₆)alkyl,
         (C₃-C₇)cycloalkyl,
         aryl,
         heterocyclo,
         heteroaryl, or 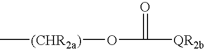
         or —(CHR₂ₐ)ₙ—Y, wherein R₂ₐ, R_{2b}, and Q are as defined above, n is an integer from 0 to 10, and Y is OH, OPO(OH)₂, OPO(O(C₁-C₆)alkyl)₂, or NR_{2d}R₂ₑ, wherein R_{2d} and R₂ₑ are each independently H, (C₁-C₆)alkyl, or (C₃-C₇)cycloalkyl, 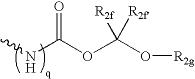
         wherein Q is O or 1, R_{2f} and R_{2f′} are each independently H, (C₁-C₆)alkyl, aryl, or heteroaryl, or taken together with the carbon to which they are attached form a 3, 4, 5, or 6 membered ring, and R_{2g} is
         (C₁-C₆)alkyl,
         (C₃-C₇)cycloalkyl,
         aryl, or
         heterocyclo, or
         heteroaryl;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
at least one of Rₐ, R_{b}, R_{c}, R_{d}, Rₑ, R_{f}, and R_{g} is
   OH,
   OPO(OH)₂,
   OPO(O(C₁-C₆)alkyl)₂, 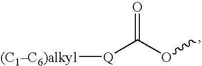
   wherein “ 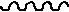
   ” indicates the point of attachment and Q is O or is absent 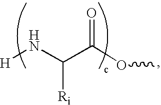
   wherein “ 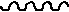
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)hydroxyalkyl
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 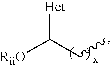
   wherein “ 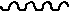
   ” indicates the point of attachment, het is a 5- or 6-membered heterocyclo or heteroaryl group, and x is an integer of from 0 to 10; 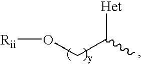
   wherein “ 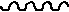
   ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂ 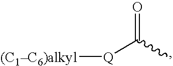
         as defined above, or 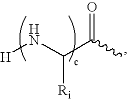
         as defined above;
and the others of Rₐ, R_{b}, R_{c}, R_{d}, Rₑ, R_{f}, and R_{g} are each independently H,
   H,
   (C₁-C₆)alkyl,
   OH,
   OPO(OH)₂,
   OPO(O(C₁-C₆)alkyl)₂ 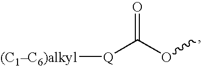
   wherein “ 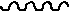
   ” indicates the point of attachment and Q is O or is absent 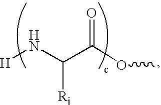
   wherein “ 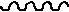
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 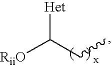
   wherein “ 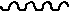
   ” indicates the point of attachment, het is a 5- or 6-membered heterocyclo or heteroaryl group, and x is an integer of from 0 to 10; 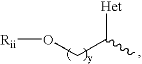
   wherein “ 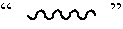
   ” indicates the point of attachment, het is as defined above, and y is an integer of from 1 to 10; wherein Rᵢᵢ is H,
      (C₁-C₆)alkyl,
      PO(OH)₂,
      PO(O(C₁-C₆)alkyl)₂ 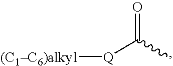
      as defined above, or 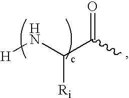
      as defined above; or
   R_{c} and Rₑ, together with the carbons to which they are attached, form a substituted or unsubstituted 4, 5, or 6-membered ring containing 0, 1, 2, or 3 heteroatoms selected from P, NH, N(C₁-C₆)alkyl, S, or O; or
   R_{c} and R_{d} or Rₑ and R_{f}, independently, together with the carbon to which they are attached, form a substituted or unsubstituted 3, 4, 5, or 6-membered ring containing 0, 1, 2, or 3 heteroatoms selected from P, NH, N(C₁-C₆)alkyl S, or O.

What is also provided is a compound which is 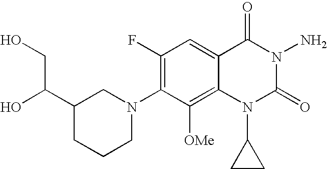
3-Amino-1-cyclopropyl-7-[3-(1,2-dihydroxy-ethyl)-piperidin-1-yl]-6-fluoro-8-methoxy-1H-quinazoline-2,4-dione; 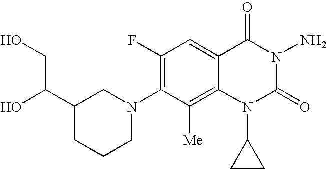
3-Amino-1-cyclopropyl-7-[3-(1,2-dihydroxy-ethyl)-piperidin-1-yl]-6-fluoro-8-methyl-1H-quinazoline-2,4-dione; 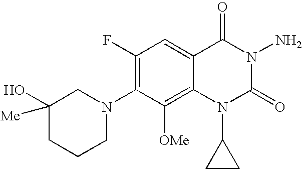
3-Amino-1-cyclopropyl-6-fluoro-7-(3-hydroxy-3-methyl-piperidin-1-yl)-8-methoxy-1H-quinazoline-2,4-dione; 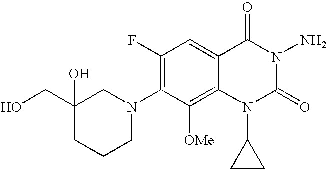
3-Amino-1-cyclopropyl-6-fluoro-7-(3-hydroxy-3-hydroxymethyl-piperidin-1-yl)-8-methoxy-1H-quinazoline-2,4-dione; 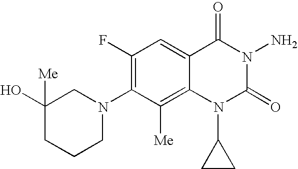
3-Amino-1-cyclopropyl-6-fluoro-7-(3-hydroxy-3-methyl-piperidin-1-yl)-8-methyl-1H-quinazoline-2,4-dione; 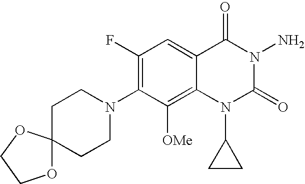
3-Amino-1-cyclopropyl-7-(1,4-dioxa-8-aza-spiro[4.5]dec-8-yl)-6-fluoro-8-methoxy-1H-quinazoline-2,4-dione; 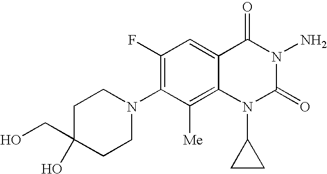
3-Amino-1-cyclopropyl-6-fluoro-7-(4-hydroxy-4-hydroxymethyl-piperidin-1-yl)-8-methyl-1H-quinazoline-2,4-dione; 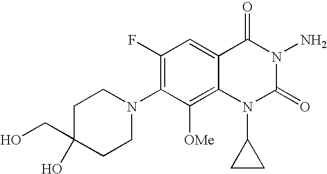
3-Amino-1-cyclopropyl-6-fluoro-7-(4-hydroxy-4-hydroxymethyl-piperidin-1-yl)-8-methoxy-1H-quinazoline-2,4-dione; 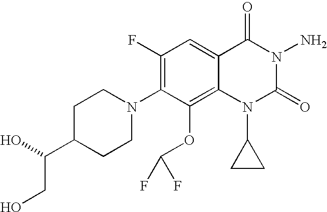
3-Amino-1-cyclopropyl-8-difluoromethoxy-7-[4-(1,2-dihydroxy-ethyl)-piperidin-1-yl]-6-fluoro-1H-quinazoline-2,4-dione; 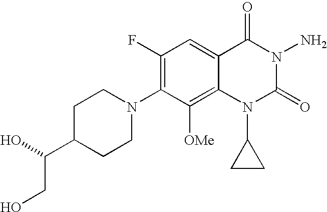
3-Amino-1-cyclopropyl-7-[4-(1,2-dihydroxy-ethyl)-piperidin-1-yl]-6-fluoro-8-methoxy-1H-quinazoline-2,4-dione; 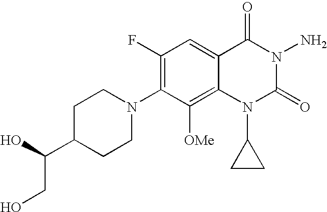
3-Amino-1-cyclopropyl-7-[4-(1,2-dihydroxy-ethyl)-piperidin-1-yl]-6-fluoro-8-methoxy-1H-quinazoline-2,4-dione; 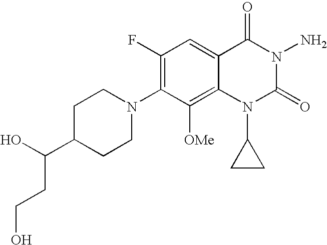
3-Amino-1-cyclopropyl-7-[4-(1,3-dihydroxy-propyl)-piperidin-1-yl]-6-fluoro-8-methoxy-1H-quinazoline-2,4-dione; 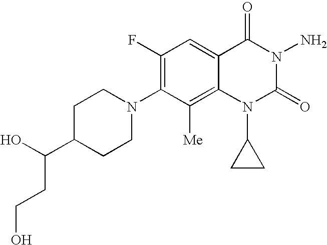
3-Amino-1-cyclopropyl-7-[4-(1,3-dihydroxy-propyl)-piperidin-1-yl]-6-fluoro-8-methyl-1H-quinazoline-2,4-dione; 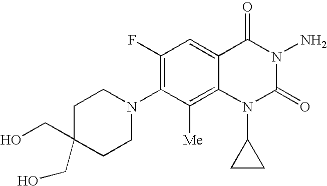
3-Amino-7-(4,4-bis-hydroxymethyl-piperidin-1-yl)-1-cyclopropyl-6-fluoro-8-methyl-1H-quinazoline-2,4-dione; 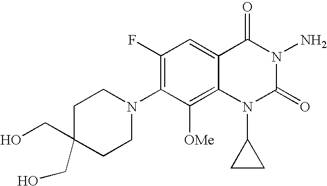
3-Amino-7-(4,4-bis-hydroxymethyl-piperidin-1-yl)-1-cyclopropyl-6-fluoro-8-methoxy-1H-quinazoline-2,4-dione; 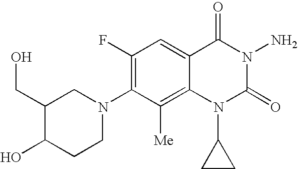
3-Amino-1-cyclopropyl-6-fluoro-7-(4-hydroxy-3-hydroxymethyl-piperidin-1-yl)-8-methyl-1H-quinazoline-2,4-dione; 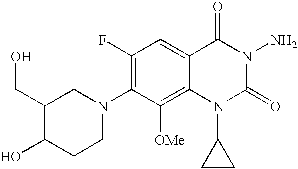
3-Amino-1-cyclopropyl-6-fluoro-7-(4-hydroxy-3-hydroxymethyl-piperidin-1-yl)-8-methoxy-1H-quinazoline-2,4-dione; 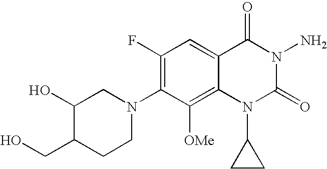
3-Amino-1-cyclopropyl-6-fluoro-7-(3-hydroxy-4-hydroxymethyl-piperidin-1-yl)-8-methoxy-1H-quinazoline-2,4-dione; 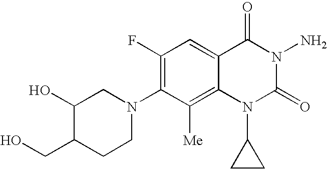
3-Amino-1-cyclopropyl-6-fluoro-7-(3-hydroxy-4-hydroxymethyl-piperidin-1-yl)-8-methyl-1H-quinazoline-2,4-dione; 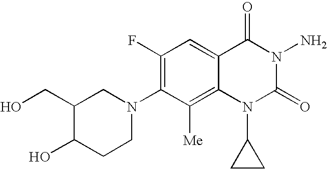
3-Amino-1-cyclopropyl-6-fluoro-7-(4-hydroxy-3-hydroxymethyl-piperidin-1-yl)-8-methyl-1H-quinazoline-2,4-dione; 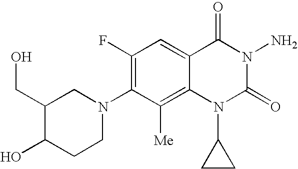
3-Amino-1-cyclopropyl-6-fluoro-7-(4-hydroxy-3-hydroxymethyl-piperidin-1-yl)-8-methyl-1H-quinazoline-2,4-dione; 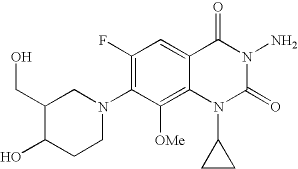
3-Amino-1-cyclopropyl-6-fluoro-7-(4-hydroxy-3-hydroxymethyl-piperidin-1-yl)-8-methoxy-1H-quinazoline-2,4-dione; 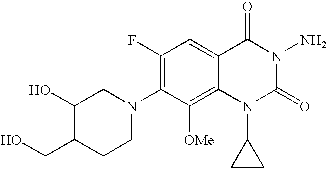
3-Amino-1-cyclopropyl-6-fluoro-7-(3-hydroxy-4-hydroxymethyl-piperidin-1-yl)-8-methoxy-1H-quinazoline-2,4-dione; 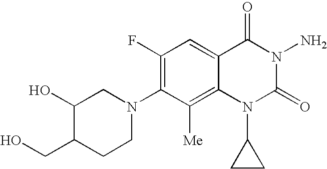
3-Amino-1-cyclopropyl-6-fluoro-7-(3-hydroxy-4-hydroxymethyl-piperidin-1-yl)-8-methyl-1H-quinazoline-2,4-dione; 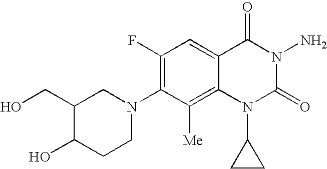
3-Amino-1-cyclopropyl-6-fluoro-7-(3-hydroxy-4-hydroxymethyl-piperidin-1-yl)-8-methyl-1H-quinazoline-2,4-dione;
3-Amino-1-cyclopropyl-7-[3-(1,2-dihydroxy-ethyl)-piperidin-1-yl]-8-methoxy-1H-quinazoline-2,4-dione;
3-Amino-1-cyclopropyl-7-[3-(1,2-dihydroxy-ethyl)-piperidin-1-yl]-8-methyl-1H-quinazoline-2,4-dione;
3-Amino-1-cyclopropyl-7-(3-hydroxy-3-methyl-piperidin-1-yl)-8-methoxy-1H-quinazoline-2,4-dione;
3-Amino-1-cyclopropyl-7-(3-hydroxy-3-hydroxymethyl-piperidin-1-yl)-8-methoxy-1H-quinazoline-2,4-dione;
3-Amino-1-cyclopropyl-7-(3-hydroxy-3-methyl-piperidin-1-yl)-8-methyl-1H-quinazoline-2,4-dione;
3-Amino-1-cyclopropyl-7-(1,4-dioxa-8-aza-spiro[4.5]dec-8-yl)-8-methoxy-1H-quinazoline-2,4-dione;
3-Amino-1-cyclopropyl-7-(4-hydroxy-4-hydroxymethyl-piperidin-1-yl)-8-methyl-1H-quinazoline-2,4-dione;
3-Amino-1-cyclopropyl-7-(4-hydroxy-4-hydroxymethyl-piperidin-1-yl)-8-methoxy-1H-quinazoline-2,4-dione;
3-Amino-1-cyclopropyl-8-difluoromethoxy-7-[4-(1,2-dihydroxy-ethyl)-piperidin-1-yl]-6-fluoro-1H-quinazoline-2,4-dione;
3-Amino-1-cyclopropyl-7-[4-(1,2-dihydroxy-ethyl)-piperidin-1-yl]-8-methoxy-1H-quinazoline-2,4-dione;
3-Amino-1-cyclopropyl-7-[4-(1,2-dihydroxy-ethyl)-piperidin-1-yl]-8-methoxy-1H-quinazoline-2,4-dione;
3-Amino-1-cyclopropyl-7-[4-(1,3-dihydroxy-propyl)-piperidin-1-yl]-8-methoxy-1H-quinazoline-2,4-dione;
3-Amino-1-cyclopropyl-7-[4-(1,3-dihydroxy-propyl)-piperidin-1-yl]-8-methyl-1H-quinazoline-2,4-dione;
3-Amino-7-(4,4-bis-hydroxymethyl-piperidin-1-yl)-1-cyclopropyl-8-methyl-1H-quinazoline-2,4-dione;
3-Amino-7-(4,4-bis-hydroxymethyl-piperidin-1-yl)-1-cyclopropyl-8-methoxy-1H-quinazoline-2,4-dione;
3-Amino-1-cyclopropyl-7-(4-hydroxy-3-hydroxymethyl-piperidin-1-yl)-8-methyl-1H-quinazoline-2,4-dione;
3-Amino-1-cyclopropyl-7-(4-hydroxy-3-hydroxymethyl-piperidin-1-yl)-8-methoxy-1H-quinazoline-2,4-dione;
3-Amino-1-cyclopropyl-7-(3-hydroxy-4-hydroxymethyl-piperidin-1-yl)-8-methoxy-1H-quinazoline-2,4-dione;
3-Amino-1-cyclopropyl-7-(3-hydroxy-4-hydroxymethyl-piperidin-1-yl)-8-methyl-1H-quinazoline-2,4-dione;
3-Amino-1-cyclopropyl-7-(4-hydroxy-3-hydroxymethyl-piperidin-1-yl)-8-methyl-1H-quinazoline-2,4-dione;
3-Amino-1-cyclopropyl-7-(4-hydroxy-3-hydroxymethyl-piperidin-1-yl)-8-methyl-1H-quinazoline-2,4-dione;
3-Amino-1-cyclopropyl-7-(4-hydroxy-3-hydroxymethyl-piperidin-1-yl)-8-methoxy-1H-quinazoline-2,4-dione;
3-Amino-1-cyclopropyl-7-(3-hydroxy-4-hydroxymethyl-piperidin-1-yl)-8-methoxy-1H-quinazoline-2,4-dione;
3-Amino-1-cyclopropyl-7-(3-hydroxy-4-hydroxymethyl-piperidin-1-yl)-8-methyl-1H-quinazoline-2,4-dione;
3-Amino-1-cyclopropyl-7-(3-hydroxy-4-hydroxymethyl-piperidin-1-yl)-8-methyl-1H-quinazoline-2,4-dione;
1-cyclopropyl-7-[3-(1,2-dihydroxy-ethyl)-piperidin-1-yl]-8-methoxy-1H-quinazoline-2,4-dione;
1-cyclopropyl-7-[3-(1,2-dihydroxy-ethyl)-piperidin-1-yl]-8-methyl-1H-quinazoline-2,4-dione;
1-cyclopropyl-7-(3-hydroxy-3-methyl-piperidin-1-yl)-8-methoxy-1H-quinazoline-2,4-dione;
1-cyclopropyl-7-(3-hydroxy-3-hydroxymethyl-piperidin-1-yl)-8-methoxy-1H-quinazoline-2,4-dione;
1-cyclopropyl-7-(3-hydroxy-3-methyl-piperidin-1-yl)-8-methyl-1H-quinazoline-2,4-dione;
1-cyclopropyl-7-(1,4-dioxa-8-aza-spiro[4.5]dec-8-yl)-8-methoxy-1H-quinazoline-2,4-dione;
1-cyclopropyl-7-(4-hydroxy-4-hydroxymethyl-piperidin-1-yl)-8-methyl-1H-quinazoline-2,4-dione;
1-cyclopropyl-7-(4-hydroxy-4-hydroxymethyl-piperidin-1-yl)-8-methoxy-1H-quinazoline-2,4-dione;
1-cyclopropyl-8-difluoromethoxy-7-[4-(1,2-dihydroxy-ethyl)-piperidin-1-yl]-6-fluoro-1H-quinazoline-2,4-dione;
1-cyclopropyl-7-[4-(1,2-dihydroxy-ethyl)-piperidin-1-yl]-8-methoxy-1H-quinazoline-2,4-dione;
1-cyclopropyl-7-[4-(1,2-dihydroxy-ethyl)-piperidin-1-yl]-8-methoxy-1H-quinazoline-2,4-dione;
1-cyclopropyl-7-[4-(1,3-dihydroxy-propyl)-piperidin-1-yl]-8-methoxy-1H-quinazoline-2,4-dione;
1-cyclopropyl-7-[4-(1,3-dihydroxy-propyl)-piperidin-1-yl]-8-methyl-1H-quinazoline-2,4-dione;
7-(4,4-bis-hydroxymethyl-piperidin-1-yl)-1-cyclopropyl-8-methyl-1H-quinazoline-2,4-dione;
7-(4,4-bis-hydroxymethyl-piperidin-1-yl)-1-cyclopropyl-8-methoxy-1H-quinazoline-2,4-dione;
1-cyclopropyl-7-(4-hydroxy-3-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methyl-1H-quinazoline-2,4-dione;
1-cyclopropyl-7-(3-hydroxy-4-hydroxymethyl-piperidin-1-yl)-8-methyl-1H-quinazoline-2,4-dione;
1-cyclopropyl-7-[3-(1,2-dihydroxy-ethyl)-piperidin-1-yl]-6-fluoro-8-methoxy-1H-quinazoline-2,4-dione;
1-cyclopropyl-7-[3-(1,2-dihydroxy-ethyl)-piperidin-1-yl]-6-fluoro-8-methyl-1H-quinazoline-2,4-dione;
1-cyclopropyl-6-fluoro-7-(3-hydroxy-3-methyl-piperidin-1-yl)-8-methoxy-1H-quinazoline-2,4-dione;
1-cyclopropyl-6-fluoro-7-(3-hydroxy-3-hydroxymethyl-piperidin-1-yl)-8-methoxy-1H-quinazoline-2,4-dione;
1-cyclopropyl-6-fluoro-7-(3-hydroxy-3-methyl-piperidin-1-yl)-8-methyl-1H-quinazoline-2,4-dione;
1-cyclopropyl-7-(1,4-dioxa-8-aza-spiro[4.5]dec-8-yl)-6-fluoro-8-methoxy-1H-quinazoline-2,4-dione;
1-cyclopropyl-6-fluoro-7-(4-hydroxy-4-hydroxymethyl-piperidin-1-yl)-8-methyl-1H-quinazoline-2,4-dione;
1-cyclopropyl-6-fluoro-7-(4-hydroxy-4-hydroxymethyl-piperidin-1-yl)-8-methoxy-1H-quinazoline-2,4-dione;
1-cyclopropyl-8-difluoromethoxy-7-[4-(1,2-dihydroxy-ethyl)-piperidin-1-yl]-6-fluoro-1H-quinazoline-2,4-dione;
1-cyclopropyl-7-[4-(1,2-dihydroxy-ethyl)-piperidin-1-yl]-6-fluoro-8-methoxy-1H-quinazoline-2,4-dione;
1-cyclopropyl-7-[4-(1,2-dihydroxy-ethyl)-piperidin-1-yl]-6-fluoro-8-methoxy-1H-quinazoline-2,4-dione;
1-cyclopropyl-7-[4-(1,3-dihydroxy-propyl)-piperidin-1-yl]-6-fluoro-8-methoxy-1H-quinazoline-2,4-dione;
1-cyclopropyl-7-[4-(1,3-dihydroxy-propyl)-piperidin-1-yl]-6-fluoro-8-methyl-1H-quinazoline-2,4-dione;
7-(4,4-bis-hydroxymethyl-piperidin-1-yl)-1-cyclopropyl-6-fluoro-8-methyl-1H-quinazoline-2,4-dione;
7-(4,4-bis-hydroxymethyl-piperidin-1-yl)-1-cyclopropyl-6-fluoro-8-methoxy-1H-quinazoline-2,4-dione;
1-cyclopropyl-6-fluoro-7-(4-hydroxy-3-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methyl-1H-quinazoline-2,4-dione; or
1-cyclopropyl-6-fluoro-7-(3-hydroxy-4-hydroxymethyl-piperidin-1-yl)-8-methyl-1H-quinazoline-2,4-dione.

What is also provided is a compound which is 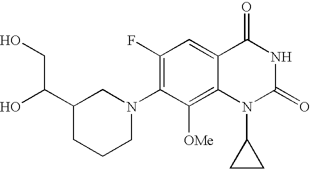
1-cyclopropyl-7-[3-(1,2-dihydroxy-ethyl)-piperidin-1-yl]-6-fluoro-8-methoxy-1H-quinazoline-2,4-dione;
1-cyclopropyl-7-[3-(1,2-dihydroxy-ethyl)-piperidin-1-yl]-6-fluoro-8-methyl-1H-quinazoline-2,4-dione;
1-cyclopropyl-6-fluoro-7-(3-hydroxy-3-methyl-piperidin-1-yl)-8-methoxy-1H-quinazoline-2,4-dione;
1-cyclopropyl-6-fluoro-7-(3-hydroxy-3-hydroxymethyl-piperidin-1-yl)-8-methoxy-1H-quinazoline-2,4-dione;
1-cyclopropyl-6-fluoro-7-(3-hydroxy-3-methyl-piperidin-1-yl)-8-methyl-1H-quinazoline-2,4-dione;
1-cyclopropyl-7-(1,4-dioxa-8-aza-spiro[4.5]dec-8-yl)-6-fluoro-8-methoxy-1H-quinazoline-2,4-dione;
1-cyclopropyl-6-fluoro-7-(4-hydroxy-4-hydroxymethyl-piperidin-1-yl)-8-methyl-1H-quinazoline-2,4-dione;
1-cyclopropyl-6-fluoro-7-(4-hydroxy-4-hydroxymethyl-piperidin-1-yl)-8-methoxy-1H-quinazoline-2,4-dione;
1-cyclopropyl-8-difluoromethoxy-7-[4-(1,2-dihydroxy-ethyl)-piperidin-1-yl]-6-fluoro-1H-quinazoline-2,4-dione;
1-cyclopropyl-7-[4-(1,2-dihydroxy-ethyl)-piperidin-1-yl]-6-fluoro-8-methoxy-1H-quinazoline-2,4-dione;
1-cyclopropyl-7-[4-(1,2-dihydroxy-ethyl)-piperidin-1-yl]-6-fluoro-8-methoxy-1H-quinazoline-2,4-dione;
1-cyclopropyl-7-[4-(1,3-dihydroxy-propyl)-piperidin-1-yl]-6-fluoro-8-methoxy-1H-quinazoline-2,4-dione;
1-cyclopropyl-7-[4-(1,3-dihydroxy-propyl)-piperidin-1-yl]-6-fluoro-8-methyl-1H-quinazoline-2,4-dione;
7-(4,4-bis-hydroxymethyl-piperidin-1-yl)-1-cyclopropyl-6-fluoro-8-methyl-1H-quinazoline-2,4-dione;
7-(4,4-bis-hydroxymethyl-piperidin-1-yl)-1-cyclopropyl-6-fluoro-8-methoxy-1H-quinazoline-2,4-dione;
1-cyclopropyl-6-fluoro-7-(4-hydroxy-3-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methyl-1H-quinazoline-2,4-dione;
1-cyclopropyl-6-fluoro-7-(3-hydroxy-4-hydroxymethyl-piperidin-1-yl)-8-methyl-1H-quinazoline-2,4-dione;
1-cyclopropyl-7-[3-(1,2-dihydroxy-ethyl)-piperidin-1-yl]-8-methoxy-1H-quinazoline-2,4-dione;
1-cyclopropyl-7-[3-(1,2-dihydroxy-ethyl)-piperidin-1-yl]-8-methyl-1H-quinazoline-2,4-dione;
1-cyclopropyl-7-(3-hydroxy-3-methyl-piperidin-1-yl)-8-methoxy-1H-quinazoline-2,4-dione;
1-cyclopropyl-7-(3-hydroxy-3-hydroxymethyl-piperidin-1-yl)-8-methoxy-1H-quinazoline-2,4-dione;
1-cyclopropyl-7-(3-hydroxy-3-methyl-piperidin-1-yl)-8-methyl-1H-quinazoline-2,4-dione;
1-cyclopropyl-7-(1,4-dioxa-8-aza-spiro[4.5]dec-8-yl)-8-methoxy-1H-quinazoline-2,4-dione;
1-cyclopropyl-7-(4-hydroxy-4-hydroxymethyl-piperidin-1-yl)-8-methyl-1H-quinazoline-2,4-dione;
1-cyclopropyl-7-(4-hydroxy-4-hydroxymethyl-piperidin-1-yl)-8-methoxy-1H-quinazoline-2,4-dione;
1-cyclopropyl-8-difluoromethoxy-7-[4-(1,2-dihydroxy-ethyl)-piperidin-1-yl]-1H-quinazoline-2,4-dione;
1-cyclopropyl-7-[4-(1,2-dihydroxy-ethyl)-piperidin-1-yl]-8-methoxy-1H-quinazoline-2,4-dione;
1-cyclopropyl-7-[4-(1,2-dihydroxy-ethyl)-piperidin-1-yl]-8-methoxy-1H-quinazoline-2,4-dione;
1-cyclopropyl-7-[4-(1,3-dihydroxy-propyl)-piperidin-1-yl]-8-methoxy-1H-quinazoline-2,4-dione;
1-cyclopropyl-7-[4-(1,3-dihydroxy-propyl)-piperidin-1-yl]-8-methyl-1H-quinazoline-2,4-dione;
7-(4,4-bis-hydroxymethyl-piperidin-1-yl)-1-cyclopropyl-8-methyl-1H-quinazoline-2,4-dione;
7-(4,4-bis-hydroxymethyl-piperidin-1-yl)-1-cyclopropyl-8-methoxy-1H-quinazoline-2,4-dione;
1-cyclopropyl-7-(4-hydroxy-3-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methyl-1H-quinazoline-2,4-dione; or
1-cyclopropyl-7-(3-hydroxy-4-hydroxymethyl-piperidin-1-yl)-8-methyl-1H-quinazoline-2,4-dione.

What is also provided is a pharmaceutical formulation comprising a compound of one of formulas I, II, III, IV, V, or VI admixed with a pharmaceutically acceptable diluent, carrier, or excipient.

What is also provided is a method of treating a bacterial infection in a mammal, comprising administering to a mammal in need thereof an effective amount of a compound of one of formulas I, II, III, IV, V, or VI.

### DETAILED DESCRIPTION OF THE INVENTION

Reference will now be made in detail to presently preferred compositions or embodiments and methods of the invention, which constitute the best modes of practicing the invention presently known to the inventors.

The term “alkyl” as used herein refers to a straight or branched hydrocarbon of from 1 to 6 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents selected from lower (C₁-C₆)alkoxy, (C₁-C₆)thioalkoxy, halogen, oxo, thio, —OH, —SH, —F, —CF₃, —OCF₃, —NO₂, —CO₂H, —CO₂(C₁-C₆)alkyl, or

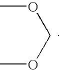

The term “(C₃-C₆)cycloalkyl” means a hydrocarbon ring containing from 3 to 6 carbon atoms, for example, cyclopropyl, cyclobutyl, cyclopentyl, or cyclohexyl. Where possible, the cycloalkyl group may contain double bonds, for example, 3-cyclohexen-1-yl. The cycloalkyl ring may be unsubstituted or substituted by one or more substituents selected from alkyl, alkoxy, thioalkoxy, hydroxy, thiol, halogen, formyl, carboxyl, —CO₂(C₁-C₆)alkyl, —CO(C₁-C₆)alkyl, aryl, heteroaryl, wherein alkyl, aryl, and heteroaryl are as defined herein, or as indicated above for alkyl. Examples of substituted cycloalkyl groups include fluorocyclopropyl.

The term “halo” includes chlorine, fluorine, bromine, and iodine.

The term “aryl” means a cyclic or polycyclic aromatic ring having from 5 to 12 carbon atoms, and being unsubstituted or substituted with one or more of the substituent groups recited above for alkyl groups including, halogen, nitro, cyano —OH, —SH, —F, —CF₃, —OCF₃, 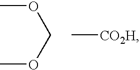
—CO₂(C₁-C₆)alkyl, or —SO₂alkyl. Examples include, but are not limited to phenyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, thienyl, naphthyl, 4-thionaphthyl, tetralinyl, anthracinyl, phenanthrenyl, benzonaphthenyl, fluorenyl, 2-acetamidofluoren-9-yl, and 4′-bromobiphenyl.

The term “heteroaryl” means an aromatic cyclic or polycyclic ring system having from 1 to 4 heteroatoms selected from N, O, and S. Typical heteroaryl groups include 2- or 3-thienyl, 2- or 3-furanyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridinyl, 3-, 4-, or 5-pyridazinyl, 2-pyrazinyl, 2-, 4-, or 5-pyrimidinyl, 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. The heteroaryl groups may be unsubstituted or substituted by 1 to 3 substituents selected from those described above for alkyl, alkenyl, and alkynyl, for example, cyanothienyl and formylpyrrolyl. Preferred aromatic fused heterocyclic rings of from 8 to 10 atoms include but are not limited to 2-, 3-, 4-, 5-, 6-, 7-, or 8-quinolinyl, 1-, 3-, 4-, 5-, 6-, 7-, or 8-isoquinolinyl-, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. Heteroaryl also includes 2- and 3-aminomethylfuran, 2- and 3-aminomethylthiophene and the like.

The term “heterocyclic” means a monocyclic, fused, bridged, or spiro bicyclic heterocyclic ring systems. Monocyclic heterocyclic rings contain from about 3 to 12 ring atoms, with from 1 to 5 heteroatoms selected from N, O, and S, and preferably from 3 to 7 member atoms, in the ring. Bicyclic heterocyclics contain from about 5 to about 17 ring atoms, preferably from 5 to 12 ring atoms. Bicyclic heterocyclic rings may be fused, spiro, or bridged ring systems. Examples of heterocyclic groups include cyclic ethers (oxiranes) such as ethyleneoxide, tetrahydrofuran, dioxane, and substituted cyclic ethers, wherein the substituents are those described above for the alkyl and cycloalkyl groups. Typical substituted cyclic ethers include propyleneoxide, phenyloxirane (styrene oxide), cis-2-butene-oxide (2,3-dimethyloxirane), 3-chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiophen-4-yl and substituted groups such as aminomethyl thiophene. Other commonly employed heterocycles include dihydro-oxathiol-4-yl, dihydro-1H-isoindole, tetrahydro-oxazolyl, tetrahydro-oxadiazolyl, tetrahydrodioxazolyl, tetrahydrooxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene.

When a bond is represented by a symbol such as “------” this is meant to represent that the bond may be absent or present provided that the resultant compound is stable and of satisfactory valency.

When a bond is represented by a line such as “ 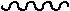
” this is meant to represent that the bond is the point of attachment between two molecular subunits.

The term “patient” means all mammals, including humans. Other examples of patients include cows, dogs, cats, goats, sheep, pigs, and rabbits.

A “therapeutically effective amount” is an amount of a compound of the present invention that, when administered to a patient, provides the desired effect; i.e., lessening in the severity of the symptoms associated with a bacterial infection.

It will be appreciated by those skilled in the art that compounds of the invention having one or more chiral centers may exist in and be isolated in optically active and racemic forms. Some compounds may exhibit polymorphism. It is to be understood that the present invention encompasses any racemic, optically-active, polymorphic, geometric, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity or cytotoxicity using the standard tests described herein, or using other similar tests which are well known in the art.

Certain compounds of Formula I are also useful as intermediates for preparing other compounds of Formula I. Thus, a compound wherein R₂ is NR₂, can be metabolized to form another compound of the invention wherein R₂ is H. This conversion can occur under physiological conditions. To that end, both the non-metabolized compound of the invention and the metabolized compound of the invention—that is, the compound wherein R₂ is NR₂ and the compound wherein R₂ is H—can have antibacterial activity.

Some of the compounds of Formula I are capable of further forming pharmaceutically acceptable acid-addition and/or base salts. All of these forms are within the scope of the present invention. Thus, pharmaceutically acceptable acid addition salts of the compounds of Formula I include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, acetate, trifluoroacetate, propionate, caprylate, isobutyrate, oxalate, malonate, succinates suberate, sebacate, fumarate, maleate, mandelate, benzoate, chlorobenzoate, methylbenzoate, dinitrobenzoate, phthalate, benzensoulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al., “Pharmaceutical Salts,” Journal of Pharmaceutical Science, 1977;66:1-19).

The acid addition salt of said basic compounds are prepared by contacting the free base form with a sufficient amount of the desired acid to produce the salt in the conventional manner.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge S. M., supra., 1977).

The base addition salts of said acidic compounds are prepared by contacting the free acid form with a sufficient amount of the desired base to produce the salt in the conventional manner.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

A “prodrug” is an inactive derivative of a drug molecule that requires a chemical or an enzymatic biotransformation in order to release the active parent drug in the body.

Specific and preferred values for the compounds of the present invention are listed below for radicals, substituents, and ranges are for illustration purposes only, and they do not exclude other defined values or other values within defined ranges for the radicals and substituents.

Thus, we turn now to a compound of formula I, which has the structure: 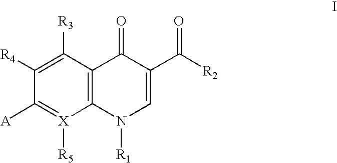
wherein A is

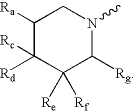

A specific value for R₁ is (C₁-C₆)cycloalkyl and halo(C₁-C₆)cycloalkyl, aryl, or heteroaryl. A specifc value for R₃ is H or NH₂. A specific value for R₄ is H or halo. A specific value for R₅ is halo, methyl, trifluoromethyl, methoxy, fluoromethoxy, difluoromethoxy, or trifluoromethoxy.

In another embodiment of a compound of formula I, a specific value for R₁ is cyclopropyl, fluorocyclopropyl, 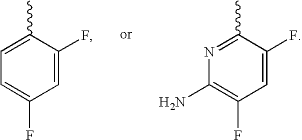
A specific value for R₃ is H or NH₂. A specific value for R₄ is H or F. A specific value for R₅ is halo, methyl, trifluoromethyl, or methoxy.

In another embodiment of a compound of formula I, specific values for R₂ is OH, O(C₁-C₆)alkyl, or OBF₂, R₄ is H or F, R₁, R₃, and R₅ are as provided in the following structures: 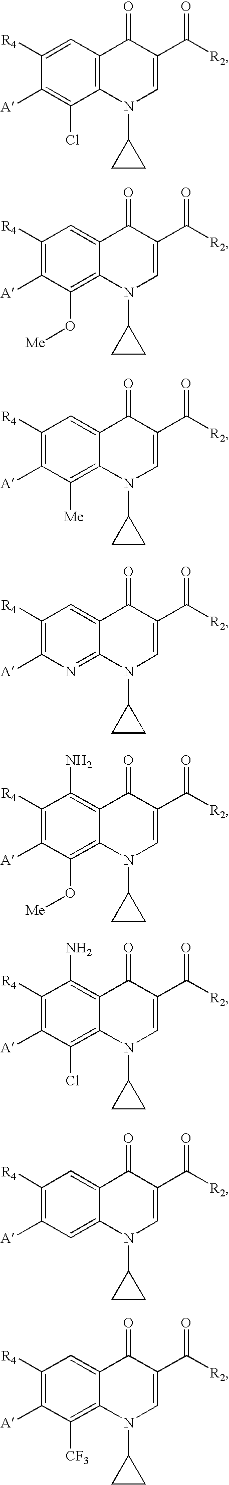 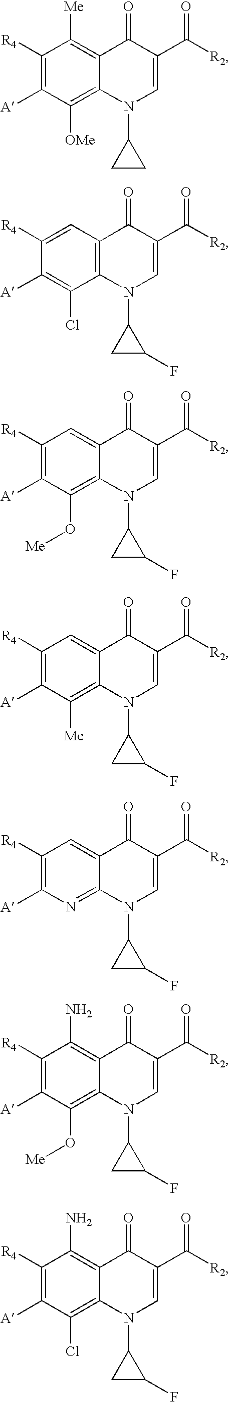 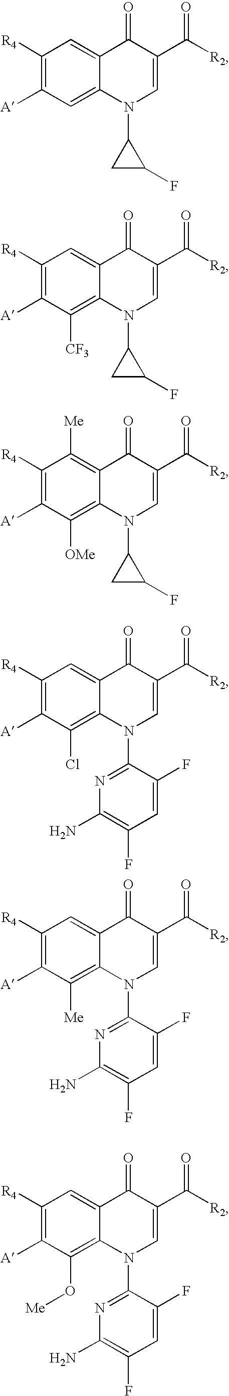 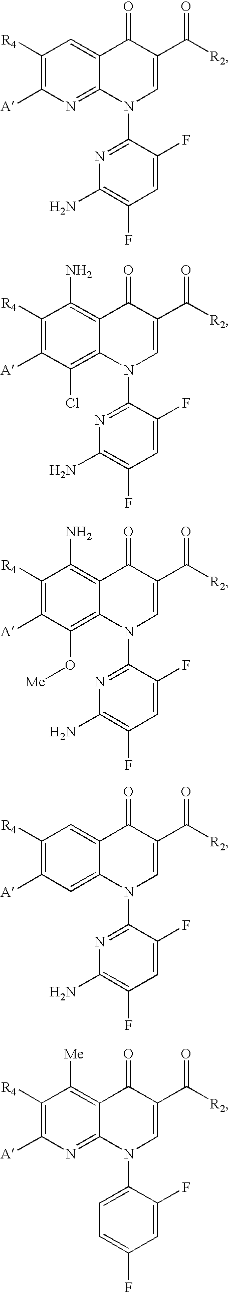 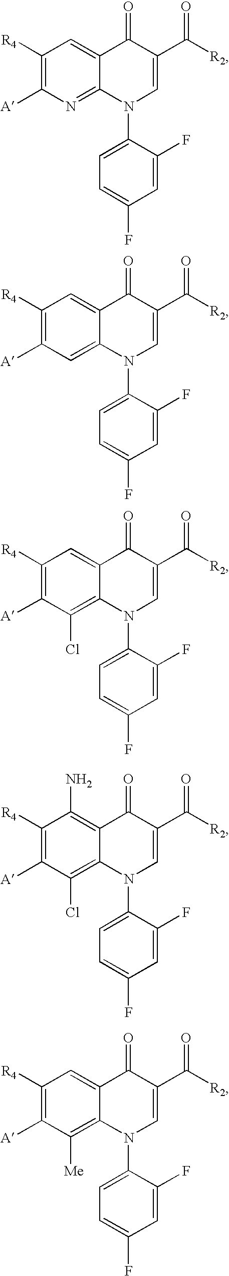 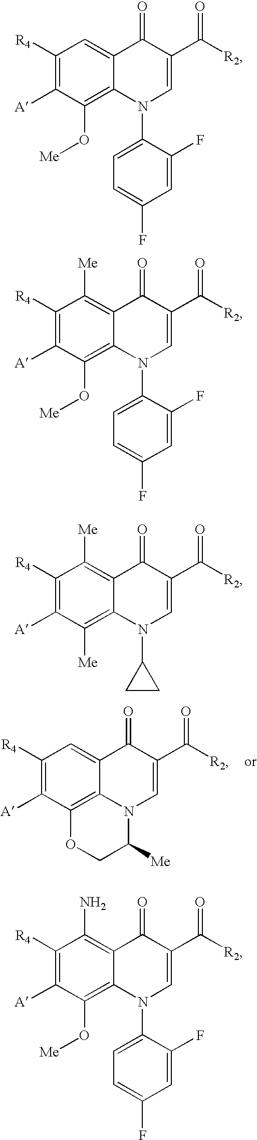
wherein A is

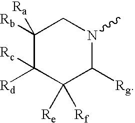

In one embodiment of a compound of formula I, R_{g} in A is H.

More particularly, when R_{g} is defined as in the previous paragraph, A is 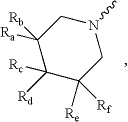
wherein “ 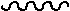
” indicates the point of attachment, and includes

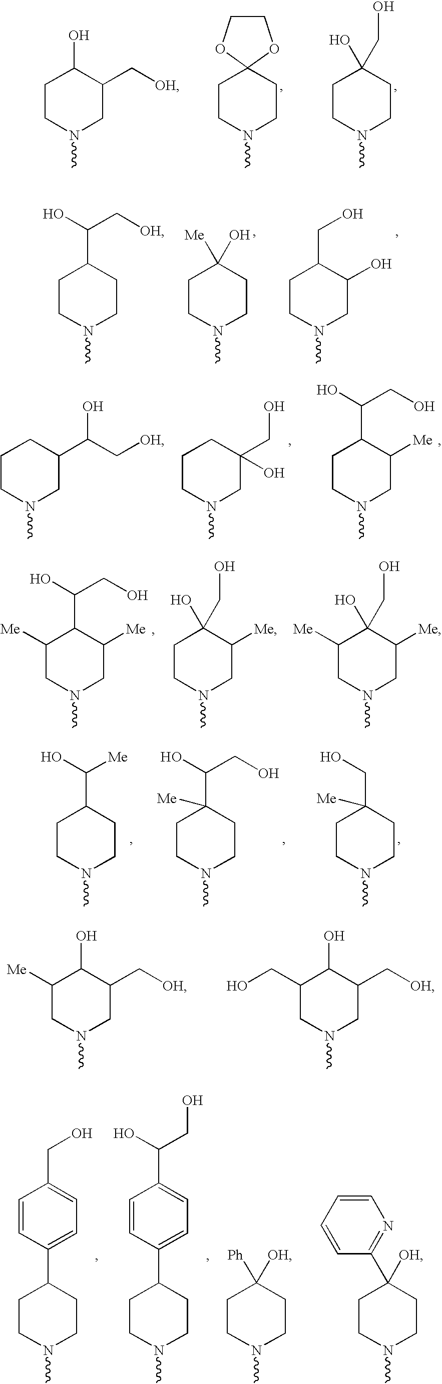

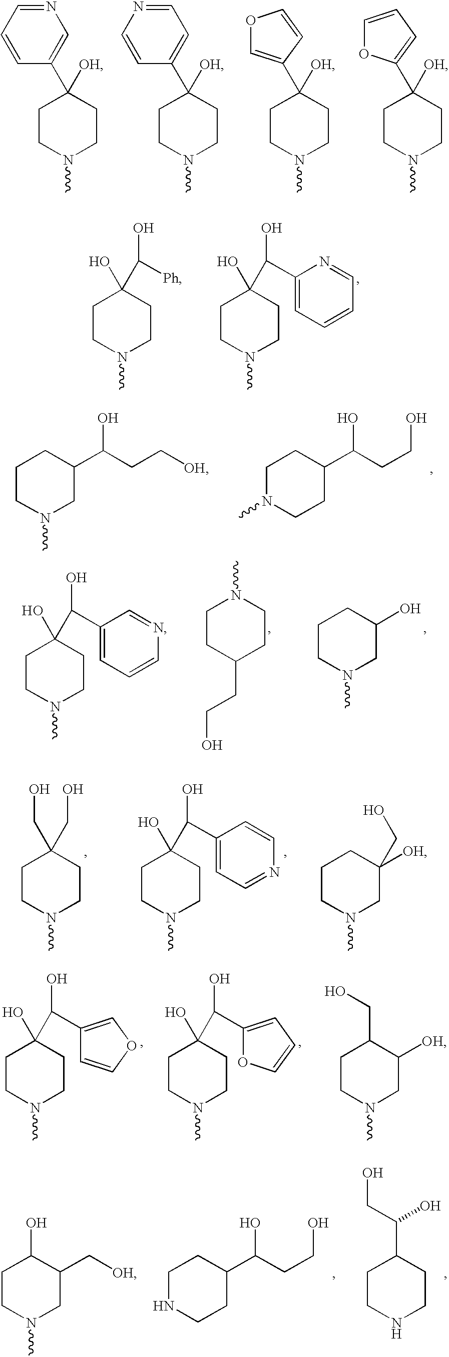

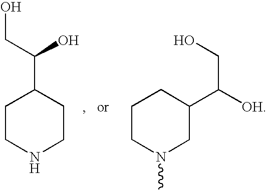

We turn now to a compound of formula II:

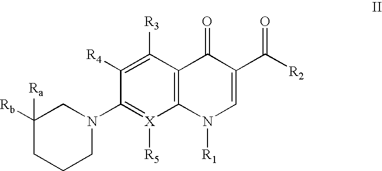

Specific values and embodiments for R₁-R₅ of compounds of formula II are as provided for compounds of formula I.

In 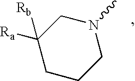
Rₐ is OH,
OPO(OH)₂,
OPO(O(C₁-C₆)alkyl)₂, 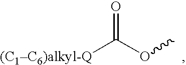
wherein “ 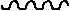
” indicates the point of attachment and Q is O or is absent 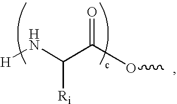
wherein “ 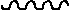
” indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)hydroxyalkyl
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 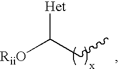
wherein “ 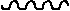
” indicates the point of attachment, het is a 5- or 6-membered heterocyclo or heteroaryl group, and x is an integer of from 0 to 10; 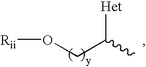
wherein “ 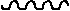
” indicates the point of attachment, het is as defined above, and y is an integer of from 1 to 10;
   wherein Rᵢᵢ is H,
      (C₁-C₆)alkyl,
      PO(OH)₂,
      PO(O(C₁-C₆)alkyl)₂, 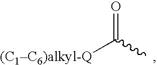
      as defined above, or 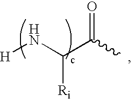
      as defined above; and
R_{b} is
   H,
   (C₁-C₆)alkyl, 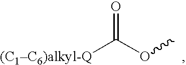
   wherein 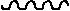
   ” indicates the point of attachment and Q is O or is absent 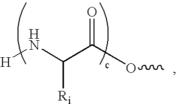
   wherein 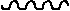
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)hydroxyalkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 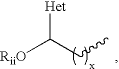
   wherein 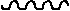
   ” indicates the point of attachment, het is a 5- or 6-membered heterocyclo or heteroaryl group, and x is an integer of from 0 to 10; 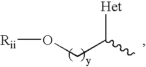
   wherein 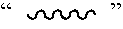
   ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, 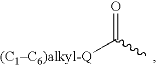
         as defined above, or 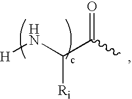
            as defined above.

More particularly 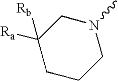
is 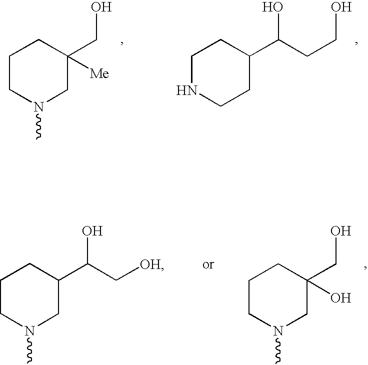
wherein 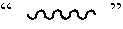
” indicates the point of attachment.

We turn now to a compound of formula III:

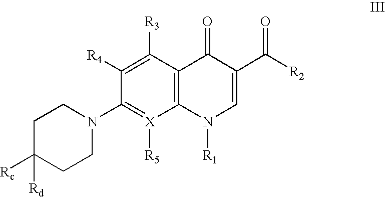

Specific values and embodiments for R₁-R₅ of compounds of formula III are as provided for compounds of formula I.

In 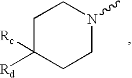
R_{c} is OH,
OPO(OH)₂,
OPO(O(C₁-C₆)alkyl)₂, 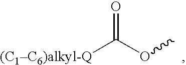
wherein 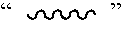
” indicates the point of attachment and Q is O or is absent 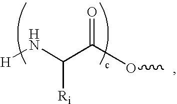
wherein 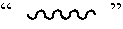
” indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)hydroxyalkyl
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 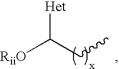
wherein 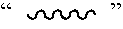
” indicates the point of attachment, het is a 5- or 6-membered heterocyclo or heteroaryl group, and x is an integer of from 0 to 10; 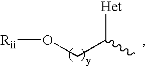
wherein 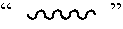
” indicates the point of attachment, het is as defined above, and y is an integer of from 1 to 10;
   wherein Rᵢᵢ is H,
      (C₁-C₆)alkyl,
      PO(OH)₂,
      PO(O(C₁-C₆)alkyl)₂, 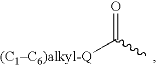
      as defined above, or 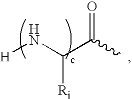
      as defined above;
and R_{d} is
   H,
   (C₁-C₆)alkyl, 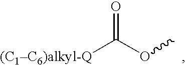
   wherein 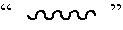
   ” indicates the point of attachment and Q is O or is absent 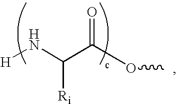
   wherein 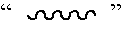
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)hydroxyalkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 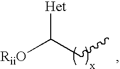
   wherein 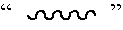
   ” indicates the point of attachment, het is a 5- or 6-membered heterocyclo or heteroaryl group, and x is an integer of from 0 to 10; 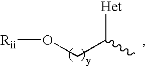
   wherein 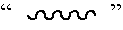
   ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, 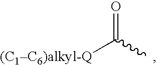
         as defined above, or 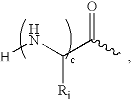
            as defined above.

More particularly 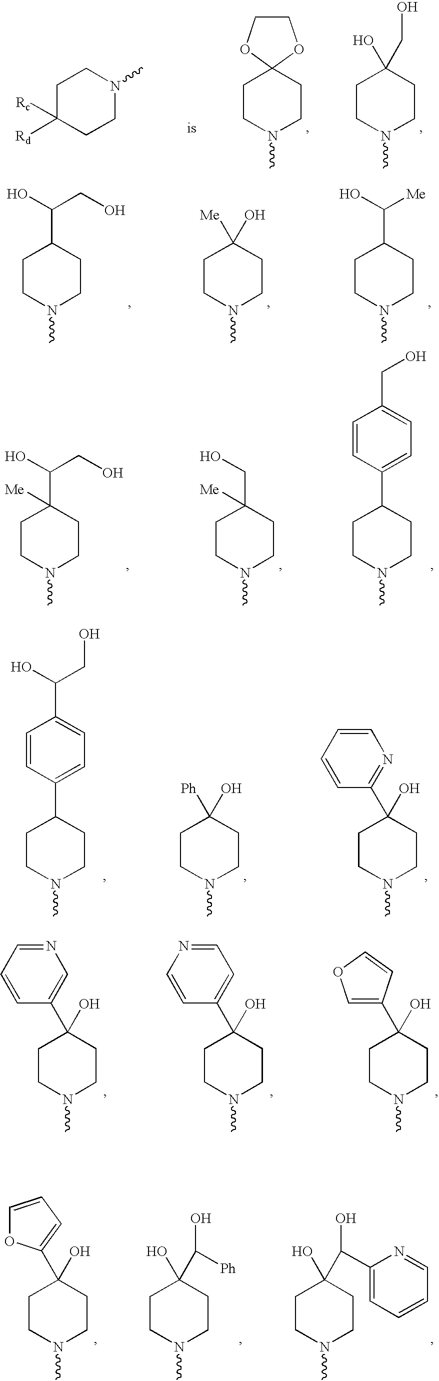 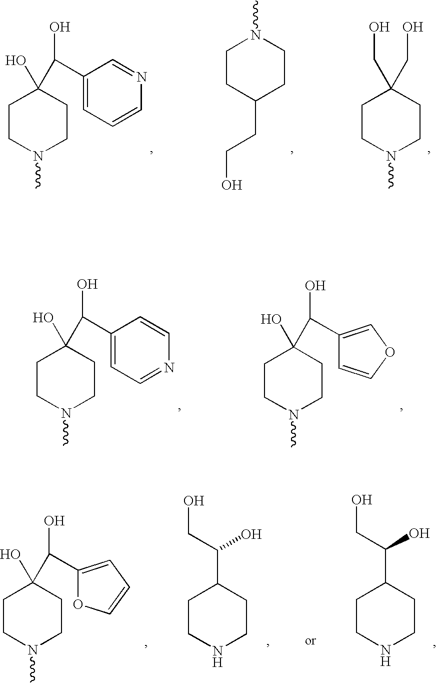
wherein 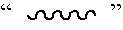
indicates the point of attachment.

We turn now to a compound of formula IV:

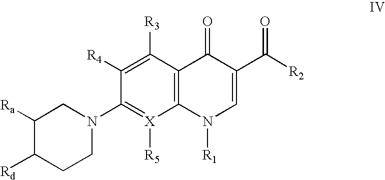

Specific values and embodiments for R₁-R₅ of compounds of formula IV are as provided for compounds of formula I.

In 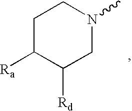
R_{c} is OH,
OPO(OH)₂,
OPO(O(C₁-C₆)alkyl)₂, 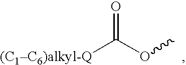
wherein 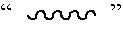
” indicates the point of attachment and Q is O or is absent 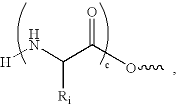
wherein 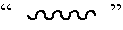
” indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)hydroxyalkyl
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 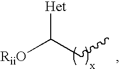
wherein 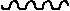
” indicates the point of attachment, het is a 5- or 6-membered heterocyclo or heteroaryl group, and x is an integer of from 0 to 10; 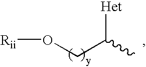
wherein 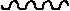
” indicates the point of attachment, het is as defined above, and y is an integer of from 1 to 10;
   wherein Rᵢᵢ is H,
      (C₁-C₆)alkyl,
      PO(OH)₂,
      PO(O(C₁-C₆)alkyl)₂, 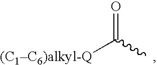
      as defined above, or 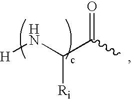
as defined above;
and R_{d} is
   (C₁-C₆)alkyl, 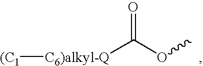
   wherein “ 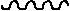
   ” indicates the point of attachment and Q is O or is absent 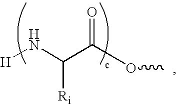
   wherein “ 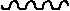
   ” indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)hydroxyalkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 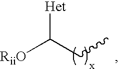
   wherein “ 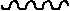
   ” indicates the point of attachment, het is a 5- or 6-membered heterocyclo or heteroaryl group, and x is an integer of from 0 to 10; 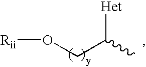
   wherein “ 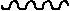
   ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, 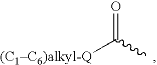
         as defined above, or 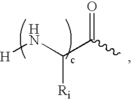
            as defined above.

More particularly, Rd is 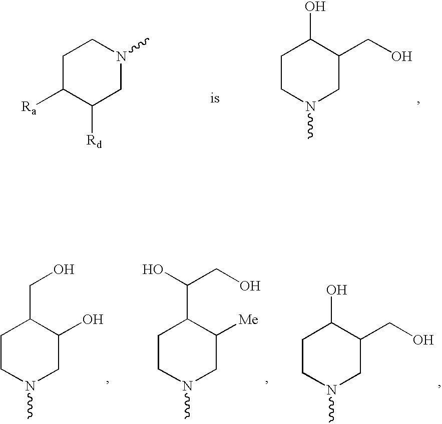
wherein “ 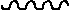
” indicates the point of attachment.

We turn now to a compound of formula V:

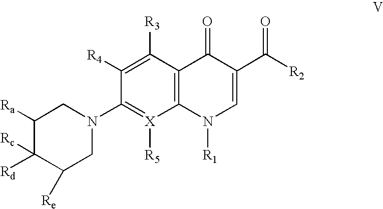

Specific values and embodiments for R₁-R₅ of compounds of formula V are as provided for compounds of formula I, except that when R₅ is OMe and R_{c} is OH, R_{d} is not H or Me.

In 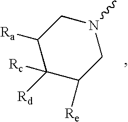
Rₐ and Rₑ are each independently (C₁-C₆)alkyl or hydroxy(C₁-C₆)alkyl;
R_{d} is H or OH; and
R_{c} is hydroxy(C₁-C₆)alkyl.

More particularly, 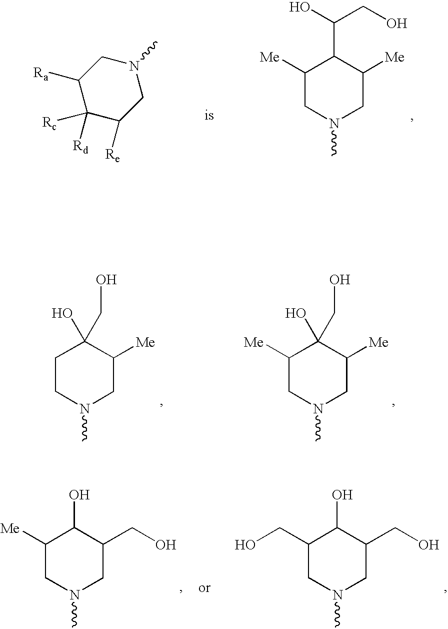
wherein “ 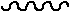
” indicates the point of attachment.

We turn now to a compound of formula VI:

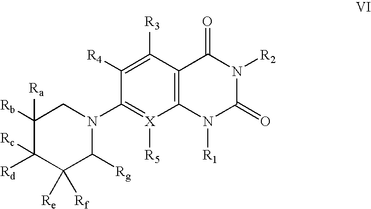

A specific value for R₁ is (C₁-C₆)cycloalkyl and halo(C₁-C₆)cycloalkyl, aryl, or heteroaryl. A specifc value for R₃ is H or NH₂. A specific value for R₄ is H or halo. A specific value for R₅ is halo, methyl, trifluoromethyl, methoxy, fluoromethoxy, difluoromethoxy, or trifluoromethoxy.

In another embodiment of a compound of formula VI, a specific value for R₁ is cyclopropyl or fluorocyclopropyl. A specific value for R₃ is H or NH₂. A specific value for R₄ is H or F. A specific value for X is C or N. A specific value for R₅ is halo or methoxy.

In another embodiment of a compound of formula VI, R₁, R₃, R₄, and R₅ are as provided in the following structures, wherein R₄ is H or F, and wherein A is

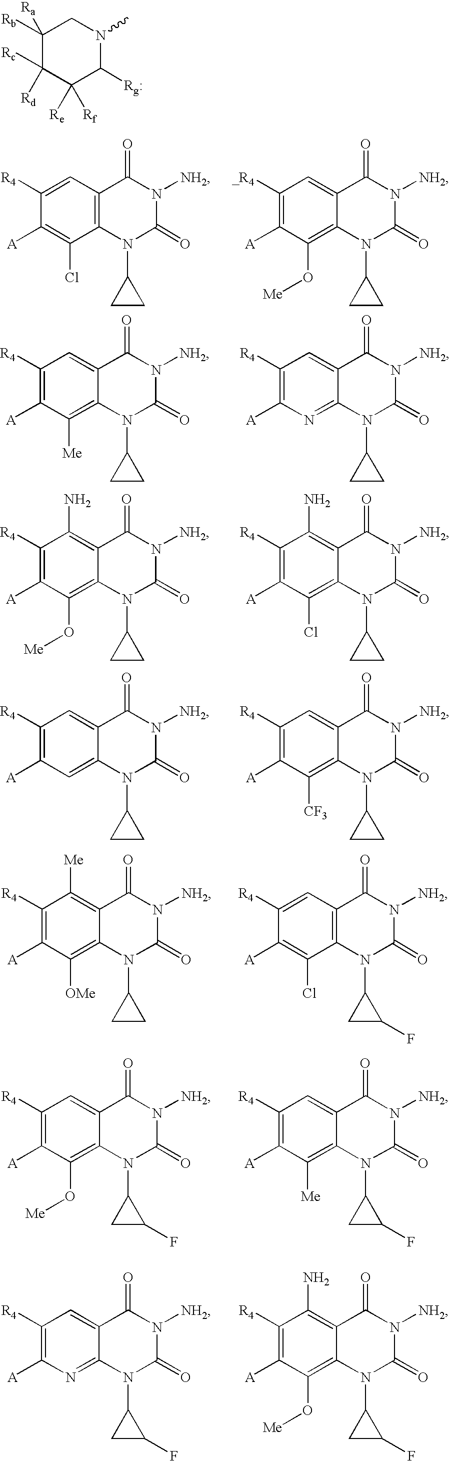

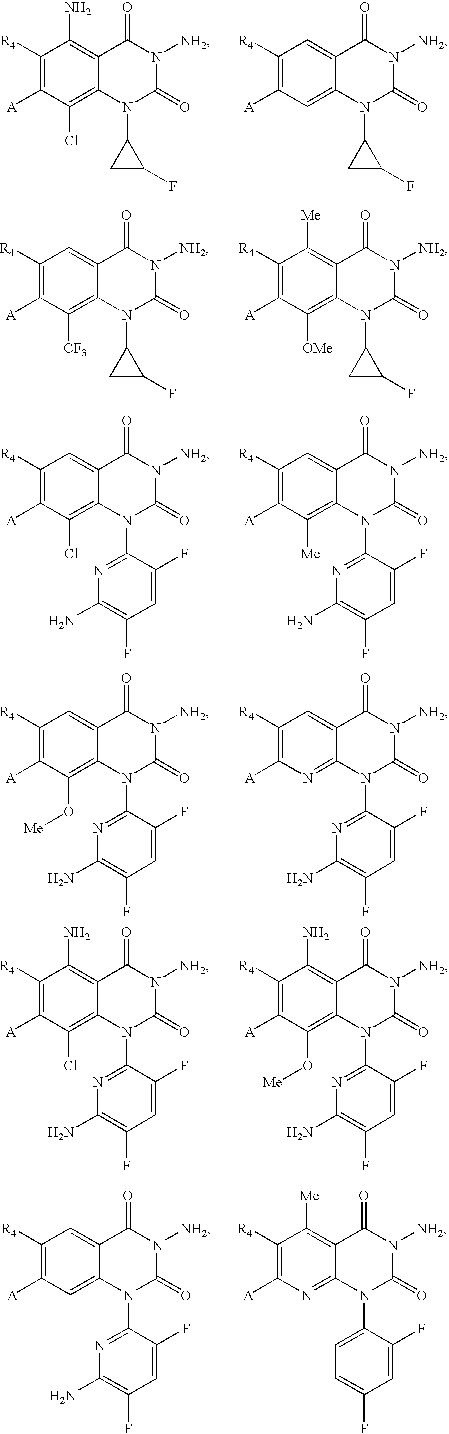

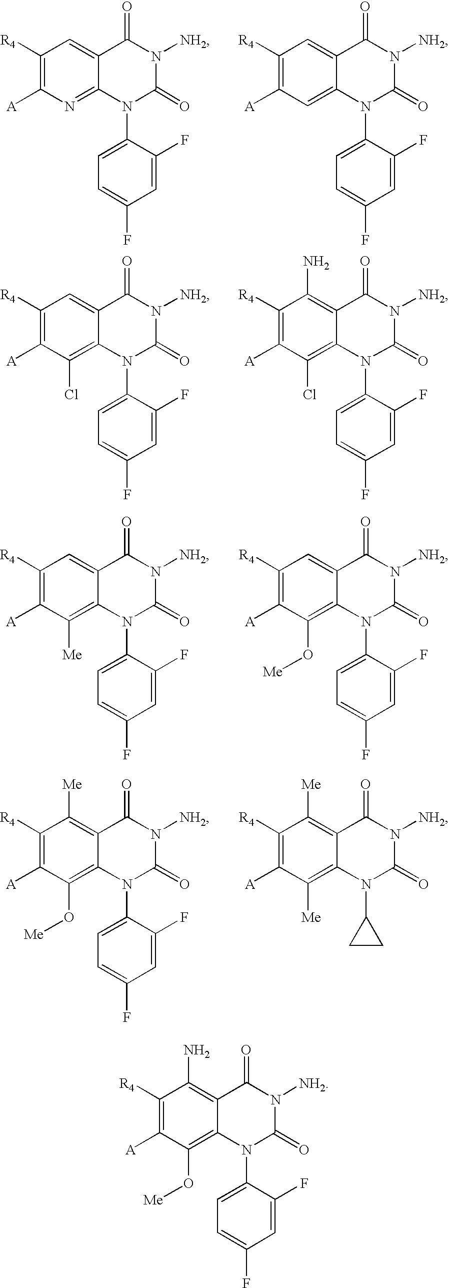

In another embodiment of a compound of formula VI, R₁, R₃, R₄, and R₅ are as provided in the following structures, wherein R₄ is H or F, and wherein A is

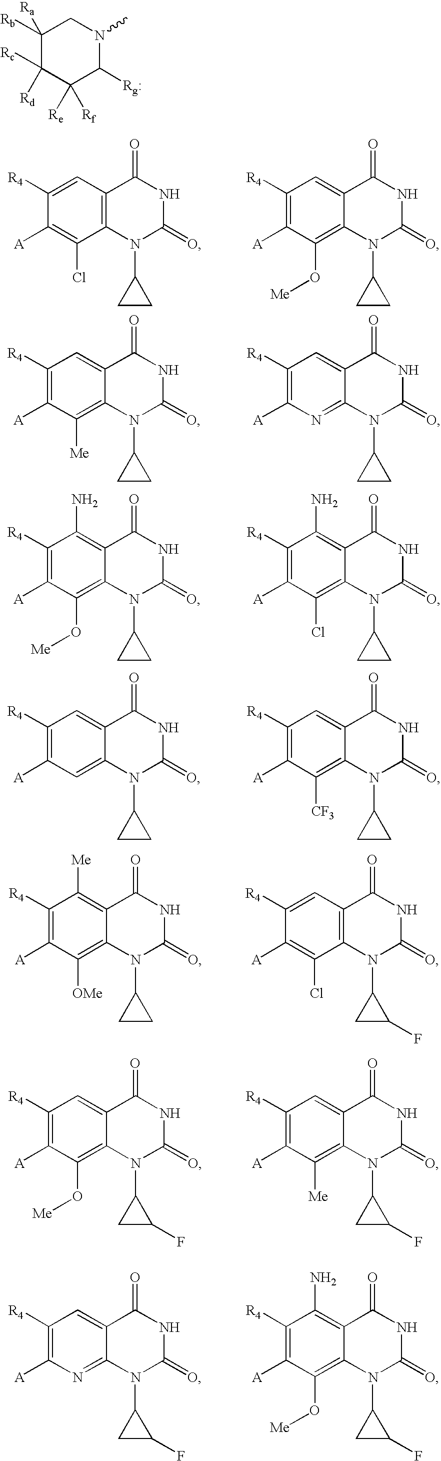

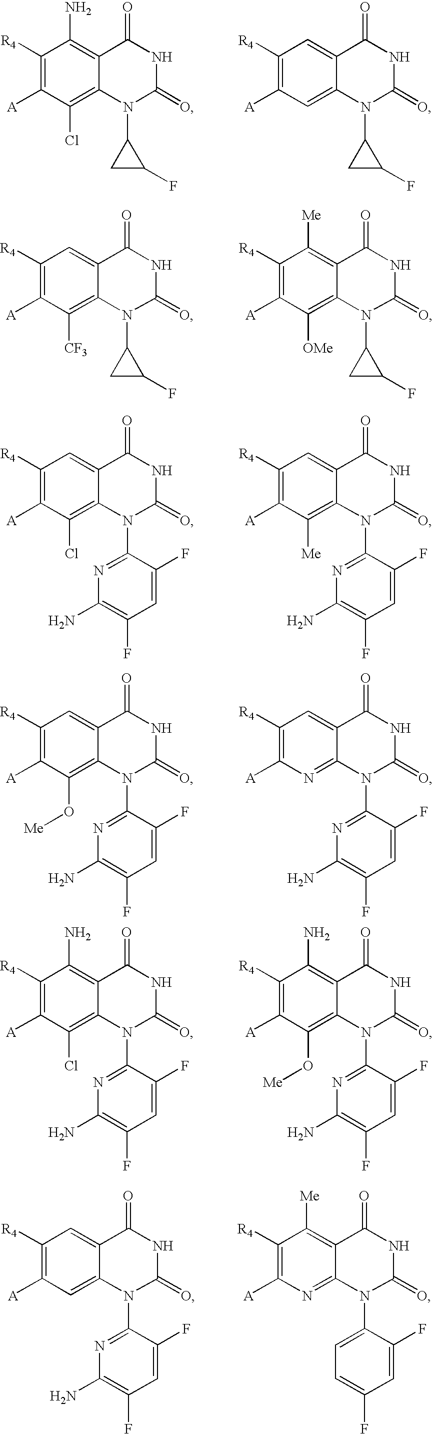

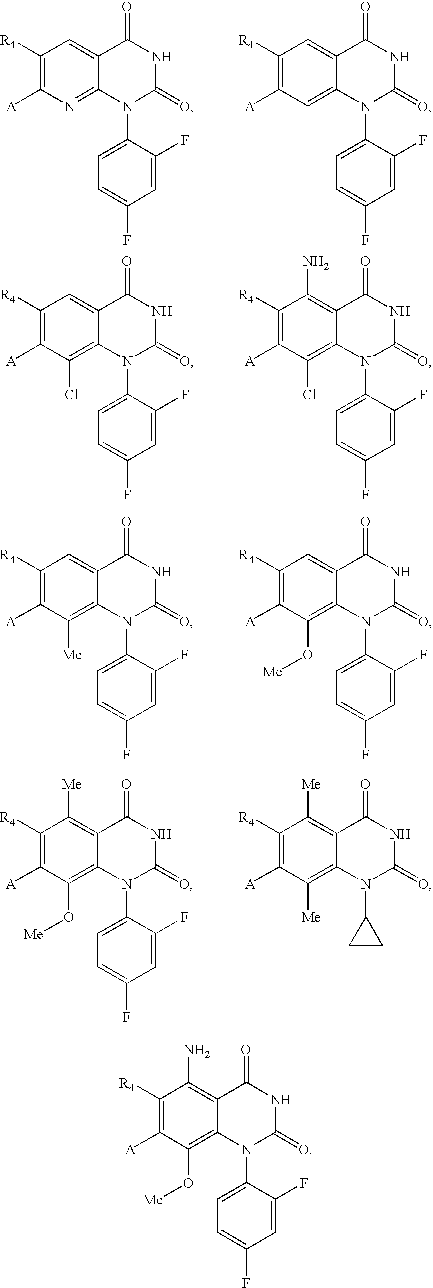

More particularly a specific value for 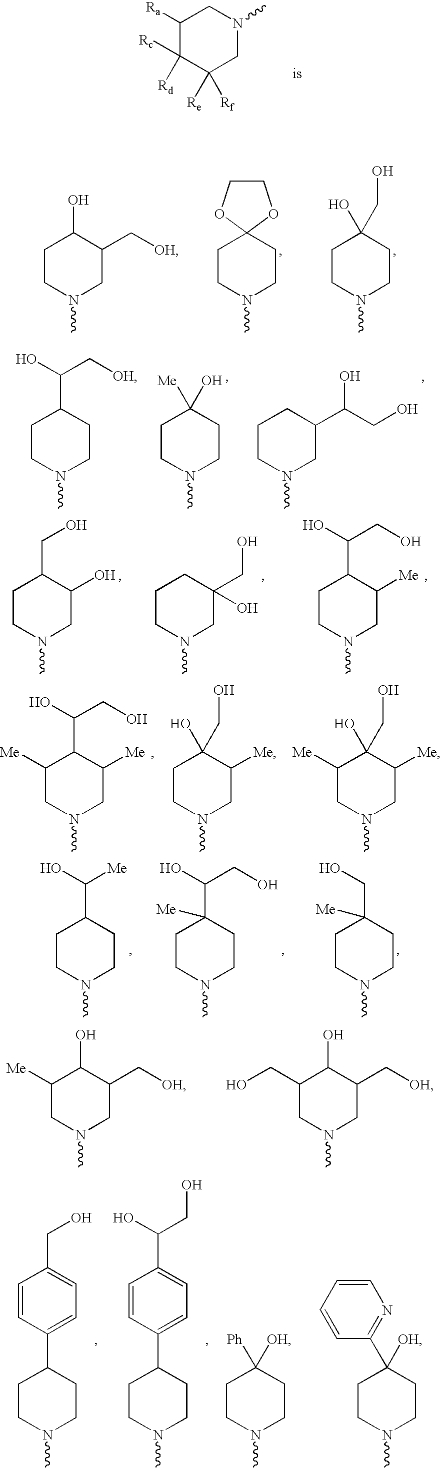 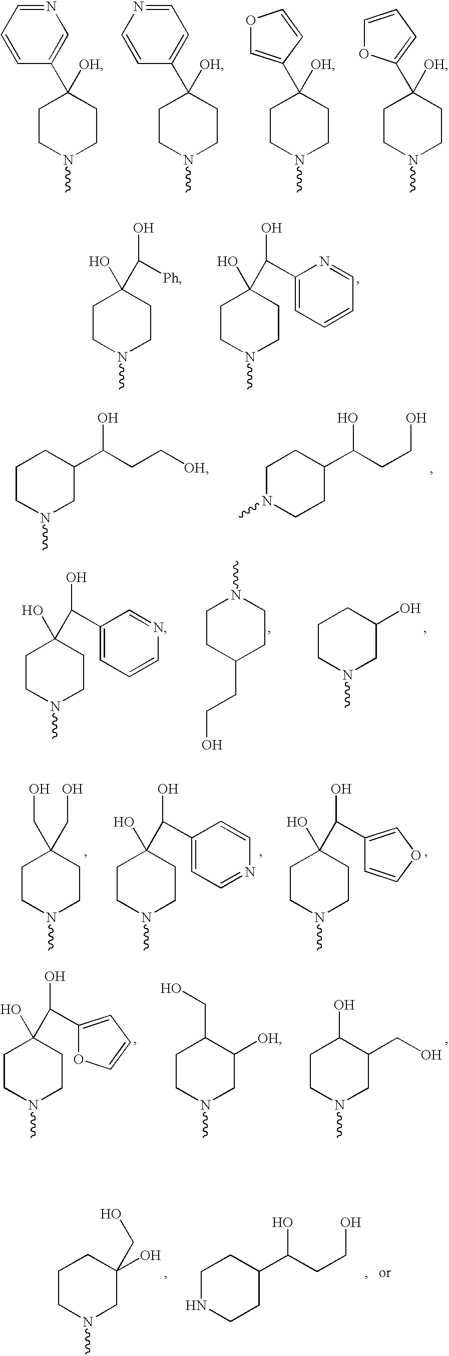 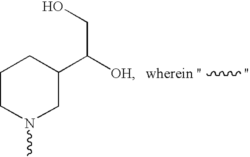
indicates the point of attachment.

### Preparation of Invention Compounds

Strategies for the preparation of invention compounds are depicted in the following schemes.

A. Invention Compounds with a Quinolone Core

As is readily apparent from this disclosure, compounds of the present invention are characterized by a quinolone core, covalently bound to an hydroxylated piperidinyl C-7 sidechain. As retrosynthetically depicted in Scheme I, the invention compounds can be prepared via coupling of a suitably C-7 substituted quinolone core precursor, wherein X is halo, triflate, or a similar reactive group known to the skilled artisan, and 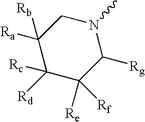
is an appropriately substituted piperidine.

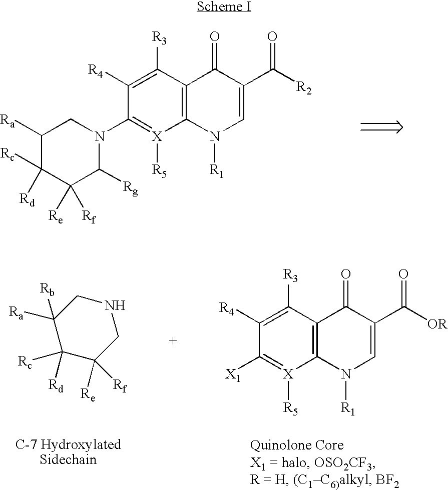

Reflecting the synthetic strategy summarized in Scheme I, the following section describing the preparation of the invention compounds has several parts. The first part describes the synthesis of the requisite quinolone core precursors. The second part describes the synthesis of the requisite C-7 sidechain precursors. The final part describes the coupling of the C-7 sidechain and quinolone core precursors to provide the invention compounds, and details any further chemical elaboration of invention compounds to produce other invention compounds.

The quinolone core precursors that are used to prepare the invention compounds are generally known to the skilled artisan and can be commercially obtained, or alternatively, can be prepared using routine synthetic methods. The following sections provide relevant citations that describe the preparation of the quinolone core precursors used to practice the invention.

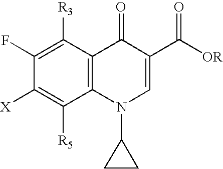

1. Preparation of Quinolone Core Precursors

| | | |
|---|---|---|
| a. | 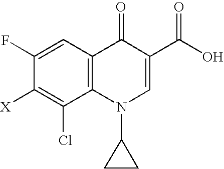 | EP 0357047; EP 0167763; EP 0195841 |
| b. | 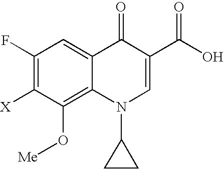 | JP8-9958 U.S. Pat. No. 5,869,991 |
| c. | 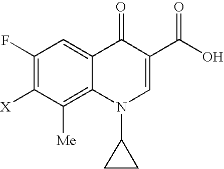 | EP 0357047 |
| d. | 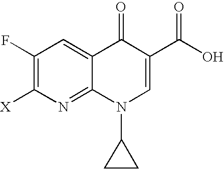 | EP 0172651 |
| e. | 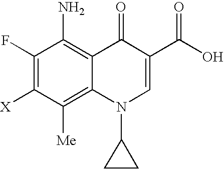 | U.S. Pat. No. 5,859,026 |
| f. | 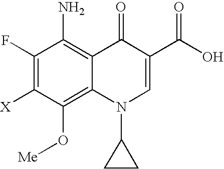 | EP 0610958 |
| g. | 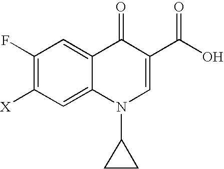 | U.S. Pat. No. 5,639,886 |

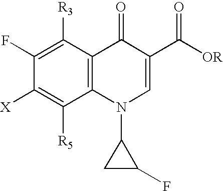 2. Preparation of Quinolone Core Precursors

| | | |
|---|---|---|
| a. | 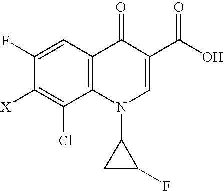 | As provided for 1A, above, except fluorocyclopropyl amine is used instead of cyclopropyl amine |
| b. | 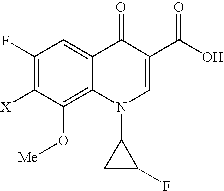 | As provided for 1A, above, except fluorocyclopropyl amine is used instead of cyclopropyl amine |
| c. | 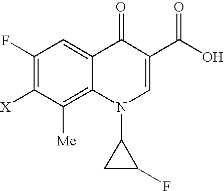 | As provided for 1C, above, except fluorocyclopropyl amine is used instead of cyclopropyl amine |
| d. | 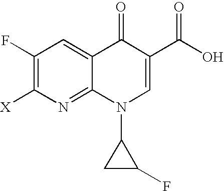 | As provided for 1D, above, except fluorocyclopropyl amine is used instead of cyclopropyl amine |
| e. | 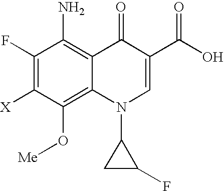 | As provided for 1F, above, except fluorocyclopropyl amine is used instead of cyclopropyl amine |
| f. | 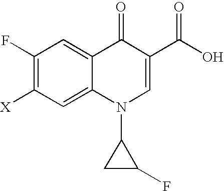 | As provided for 1H, above, except fluorocyclopropyl amine is used instead of cyclopropyl amine |

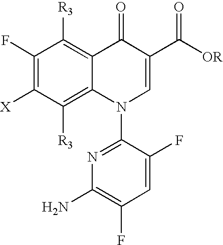 3. Preparation of

| | | |
|---|---|---|
| a. | 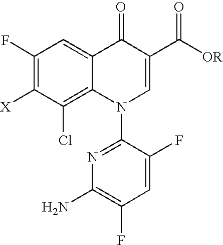 | U.S. Pat. No. 5,998436 |
| b. | 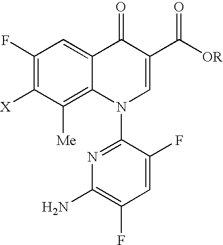 | U.S. Pat. No. 5,998436 |
| c. | 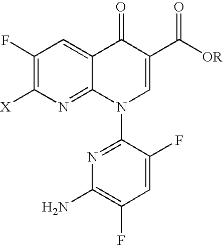 | U.S. Pat. No. 5,998436 |
| d. | 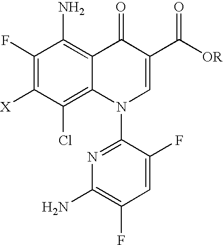 | U.S. Pat. No. 5,998436 |
| e. | 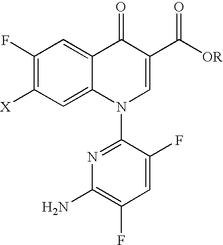 | U.S. Pat. No. 5,998436 |

B. Preparation of Quniazolinedione Core Precursors

Preparation of the requisite quinazolinedione core precursor occurs as described in WO/02 102793, priority date Jun. 19, 2001 and WO/01 53273, priority date Oct. 18, 2000, and references cited therein.

C. Synthesis of Hydroxylated C-7 Sidechain Precurors

1. Preparation of

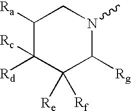

Sidechain precursor 3, 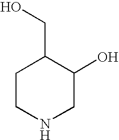
was prepared as provided in Scheme 1. LAH reduction of compound 1 provided diol 2, which was was deprotected to afford the requisite sidechain precursor, see, e.g., Col. Czech. Chem. Commun. 34, 1969, 3186-3188.

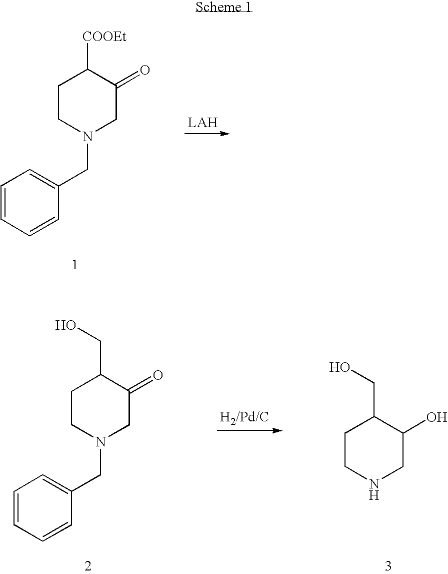

Sidechain precursor 7, 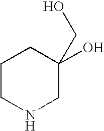
was prepared as provided in Scheme 2. Ketone 4 was converted to the exo epoxide 5 using trimethylsulfoxonium iodide in the presence of base, see, e.g., J. Hetereocyclic Chem. 1968, 5, 467-469. Treatment of 5 with aquesous acid provided diol 6, which was deprotected to afford the requisite sidechain precursor.

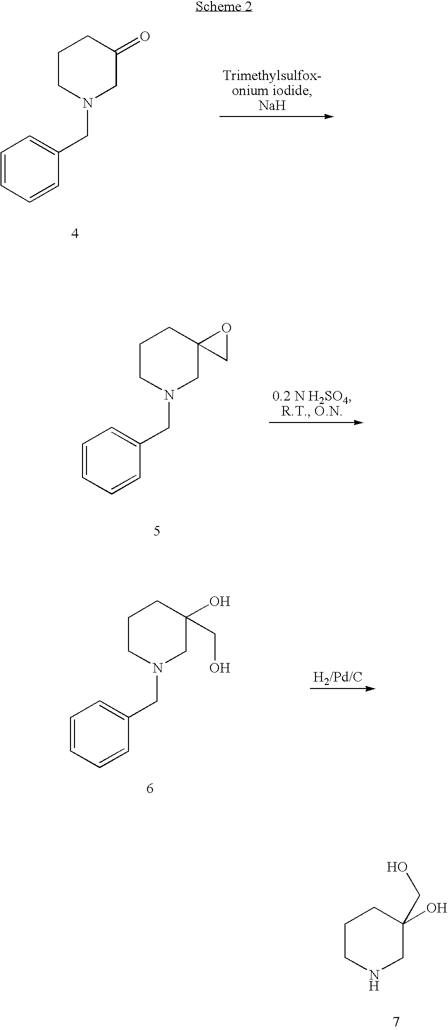

Sidechain precursor 9, 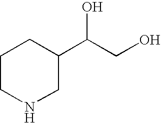
was prepared as provided in Scheme 3 Magnesium bromide was coupled to 3-bromopyridine under conventional conditions to afford 3-vinylpyridine 8. Dihydroxylation of the double bond in 8 using water admixed with t-butyl alcohol, see, e.g., Tetrahedron: Asymm. 1995, 6, 505-518, afforded the requisite sidechain precursor 9.

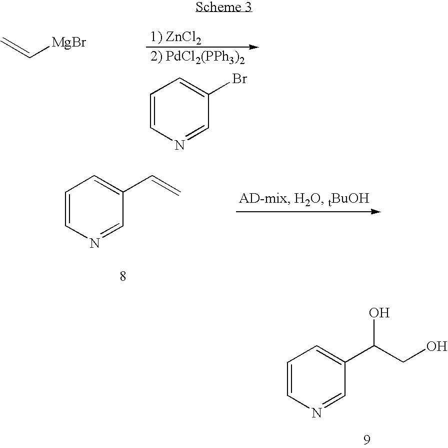

Sidechain precursor 12, 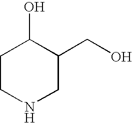
was prepared as provided in Scheme 4 and disclosed in Synthesis 1999, 1937-1943. Ketoester 10 was transesterified, reduced with sodium borohydride to provide 11, which was deprotected to afford the requisite sidechain precursor.

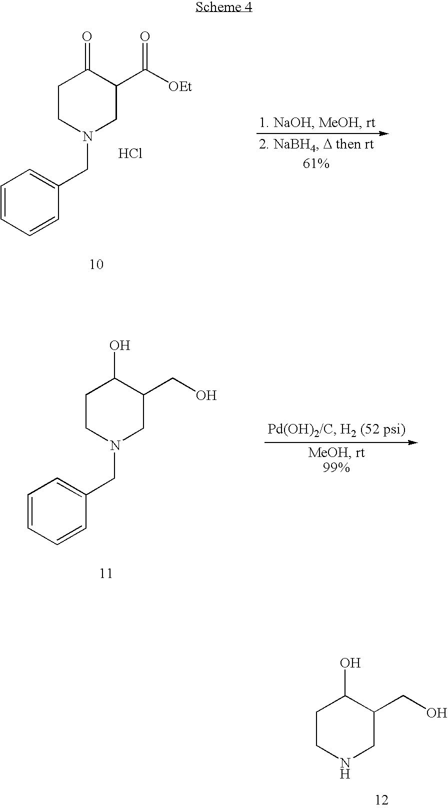

Sidechain precursor 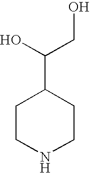
was prepared as provided in Scheme 5. Treatment of 4-vinylpyridine 13 with water admixed with t-butyl alcohol provided the diol 14. Conversoin of 14 to its hydrochloride salt 15, follwed by hydrogenation, see, e.g., J. Org. Chem. 1965, 30, 1331-1333; 1969 U.S. Pat. No. 3,464,997, provided the requisite sidechain precursor as the hydrochloride salt.

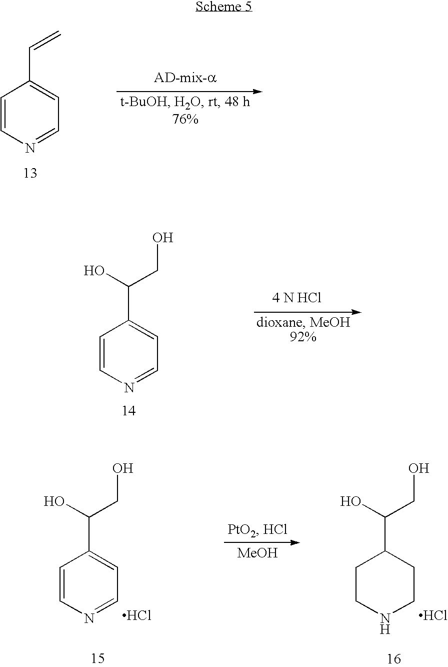

Sidechain precursor 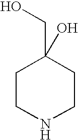
was prepared as provided in Scheme 6. Ketone 17 was converted to the exo epoxide 18 as provided in Scheme 2. Acid mediated hydrolysis of 18 provided diol 19, which was deprotected to afford the requisite sidechain.

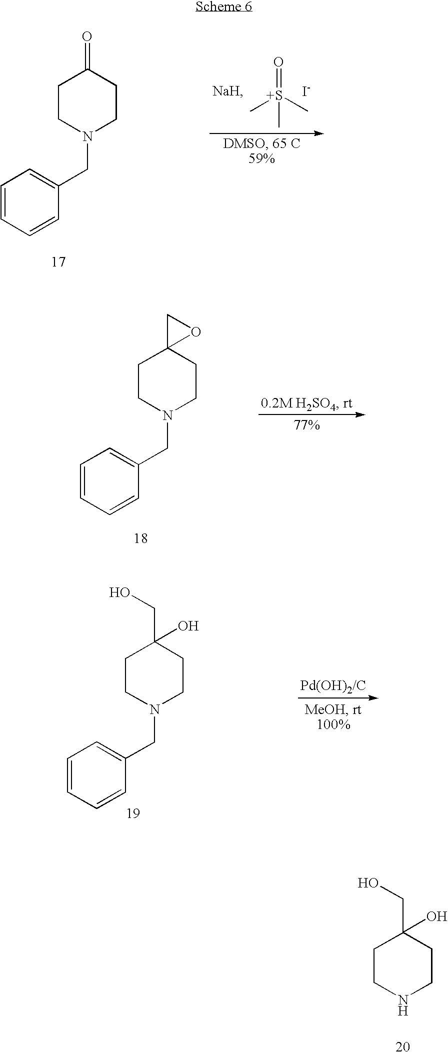

Sidechain precursor 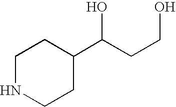
was prepared as provided in Scheme 7. Piperidine 21 was Boc-protected, and then reduced to aldehyde 22. Aldehyde 22 was alkylated to provde 23, which was reduced and deprotected to give the 4 desired compound.

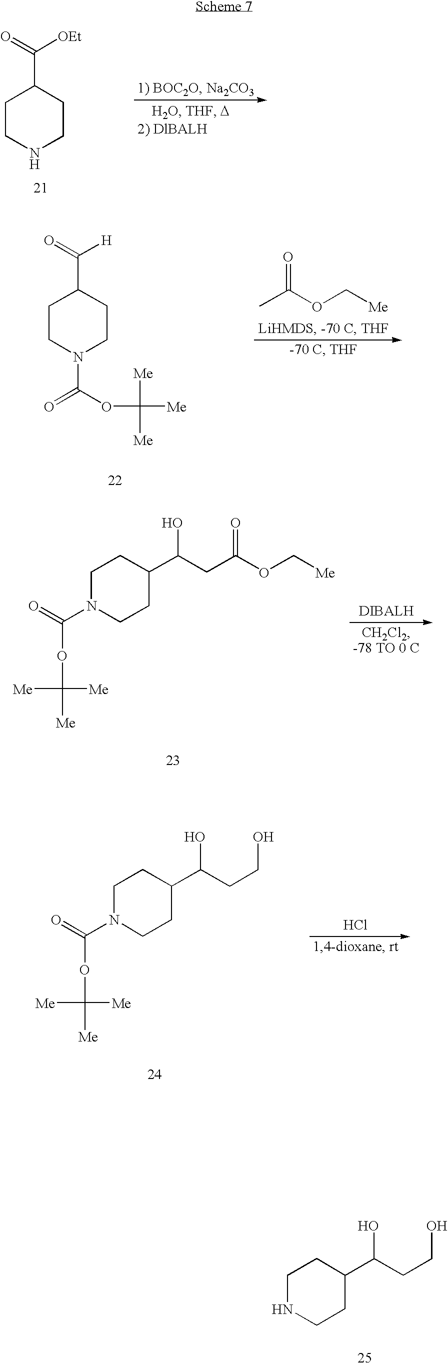

Scheme 8 discloses the synthesis of

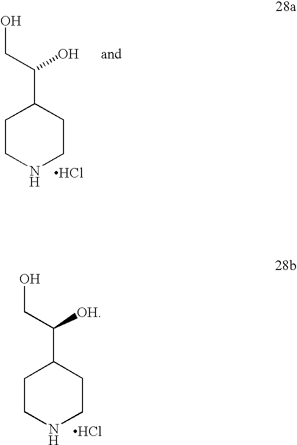

Vinylpyridine 26 was treated with either AD-mix-α or AD-mix-β to provide diol 27a or 27b. Each of the diols was submitted to hydrogenation in the presence of acid to provide the desired compounds. 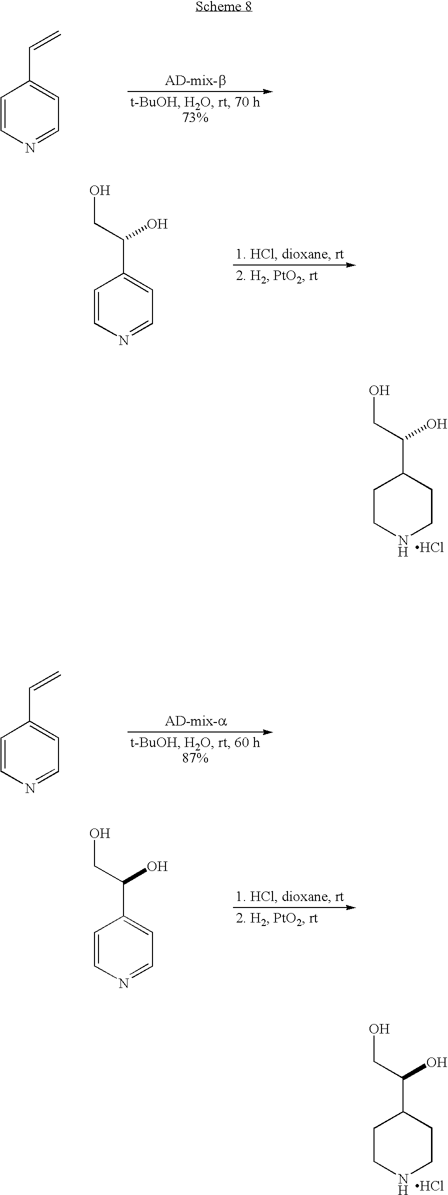
C. Coupling of Hydroxylated C-7 Sidechain and Quinolone or Quinazolinedione Core Precurors to Provide Invention Compounds

1. Coupling to Quinolone Core

Coupling of the sidechain precursor to the quinolone core precursor to provide the compounds of the present invention can occur from either the core precursor as the free acid, alkyl ester, or borate ester, as depicted in Scheme 9.

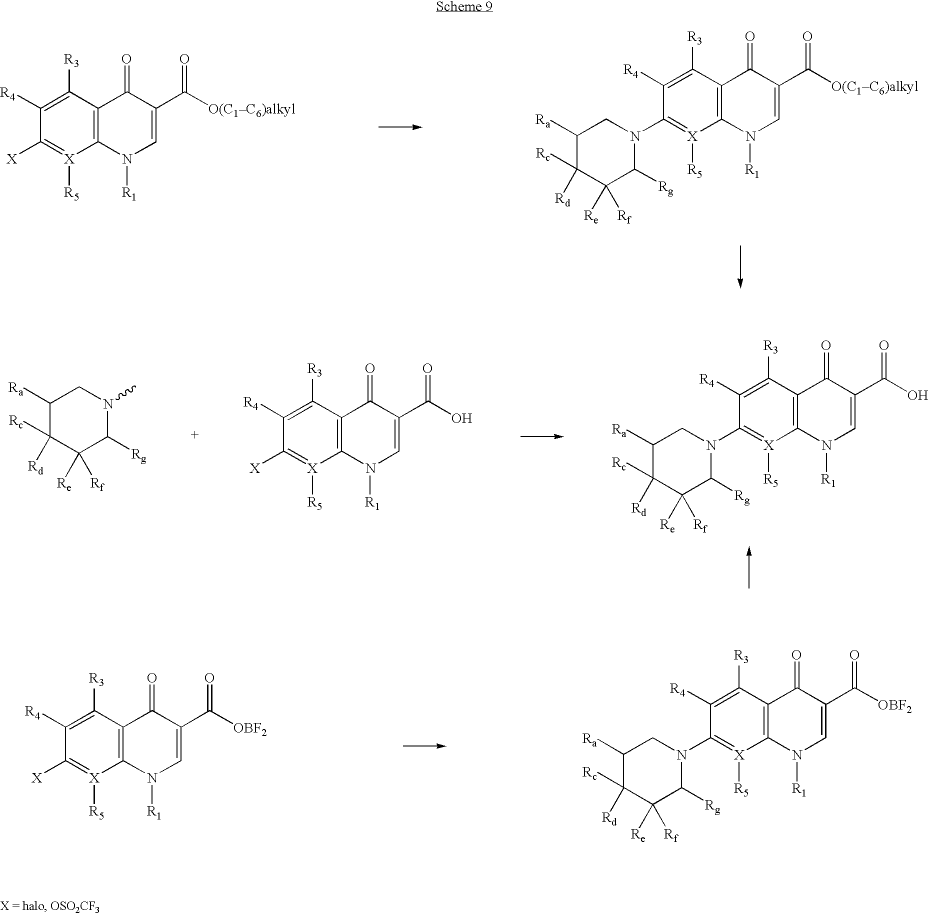

Typically, when a free acid is used in the coupling reaction, a molar excess of the side chain precursor is combined with the quinolone core in a polar solvent such as acetonitrile. A molar excess of an amine base such as triethylamine is added, and the reaction mixture is heated to about 80° C.; however, sometimes higher temperatures (140° C.) and a polar solvent (HMPA) are required. Typically, the reaction mixtures becomes homogenous. The mixture is heated for sufficient time to drive the reaction to completion, typically from about 3 to about 12 hours. The mixture is then worked up according to procedures widely uused by the skilled artisan to provide a compound of the invention.

If an alkyl ester is used in the coupling reaction, the quinolone core, sidechain, and triethylamine are combined in a solvent such as acetonitrile. The resulting reaction mixture is heated to about 80° C. and stirred for about 12 hours. Typically, the reaction mixtures becomes homogenous. The mixture is heated for sufficient time to drive the raction to completion, typically from about 3 to about 12 hours. The mixture is then worked up according to procedures widely available to the skilled artisan to provide a compound of the invention. The alky ester can then be hydrolyzed to the free acid or to an appropriate salt form according to methods that are widely available to the skilled artisan.

When a borate ester is used in the coupling reaction, the requisite borate ester is typically prepared from the free acid upon reaction with BF₃ according to conditions available to the skilled artisan. The borate ester is typically combined with the side chain in a solvent such as acetonitrile or DMSO and treated with an amine base such as triethylamine or diisopropylethyl amine. The resulting reaction mixture is typically heated at 60-90° C. for sufficient time to drive the reaction to completion, typically from about 24 to about 96 hours. The mixture is then worked up according to procedures widely used by the skilled artisan to provide a compound of the invention. The borate ester can then be hydrolyzed to the free acid or to an appropriate salt form according to methods that are widely available to the skilled artisan.

2. Coupling to Aminoquinazolinedione Core

As is readily apparent from this disclosure, other compounds of the present invention are characterized by a aminoquinazolinedione core, covalently bound to an hydroxylated piperidinyl C-7 sidechain. As retrosynthetically depicted in Scheme 10, the invention compounds can be prepared via coupling of a suitably C-7 substituted quinazolinedione core precursor, wherein X is halo, triflate, or a similar reactive group known to the skilled artisan, and 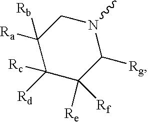
an appropriately substituted piperidine, the preparation of which are described in the previous sections.

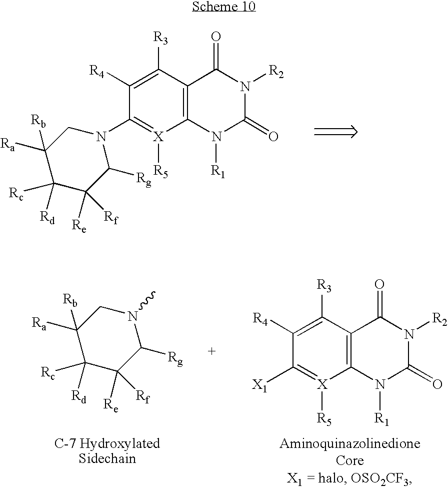

Coupling of the sidechain precursor to the quinazolinedione core precursor to provide the compounds of the present invention occurs as described in WO/02 102793, priority date Jun. 19, 2001 and WO/0153273, priority date Oct. 18, 2000, and references cited therein.

### Pharmaceutical Formulations

The present invention also provides pharmaceutical compositions which comprise a bioactive invention compound or a salt such or a pharmaceutically acceptable salt thereof and optionally a pharmaceutically acceptable carrier. The compositions include those in a form adapted for oral, topical or parenteral use and can be used for the treatment of bacterial infection in mammals including humans.

Compounds of the invention can be formulated for administration in any convenient way for use in human or veterinary medicine, by analogy with other bioactive agents such as antibiotics. Such methods are known in the art and are not described in detail herein.

The composition can be formulated for administration by any route known in the art, such as subdermal, by-inhalation, oral, topical or parenteral. The compositions may be in any form known in the art, including but not limited to tablets, capsules, powders, granules, lozenges, creams or liquid preparations, such as oral or sterile parenteral solutions or suspensions.

The topical formulations of the present invention can be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present, for example, from about 1% up to about 98% of the formulation. For example, they may form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods will known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% by weight, e.g., from about 10-60% by weight, of the active material, depending on the method of administration. Where the compositions comprise dosage units, each unit will contain, for example, from about 50-500 mg of the active ingredient. The dosage as employed for adult human treatment will range, for example, from about 100 to 3000 mg per day, for instance 1500 mg per day depending on the route and frequency of administration. Such a dosage corresponds to about 1.5 to 50 mg/kg per day. Suitably the dosage is, for example, from about 5 to 20 mg/kg per day.

### Biological Activity

The invention compounds can be screened to identify bioactive molecules with different biological activities using methods available in the art. The bioactive molecules, for example, can possess activity against a cellular target, including but not limited to enzymes and receptors, or a microorganism. A target cellular ligand or microorganism is one that is known or believed to be of importance in the etiology or progression of a disease. Examples of disease states for which compounds can be screened for biological activity include, but are not limited to, inflammation, infection, hypertension, central nervous system disorders, and cardiovascular disorders.

In one embodiment, the invention provides methods of treating or preventing a bacterial infection in a subject, such as a human or other animal subject, comprising administering an effective amount of an invention compound as disclosed herein to the subject. In one embodiment, the compound is administered in a pharmaceutically acceptable form optionally in a pharmaceutically acceptable carrier. As used herein, an “infectious disorder” is any disorder characterized by the presence of a microbial infection, such as bacterial infections. Such infectious disorders include, for example central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. The compounds and compositions comprising the compounds can be administered by routes such as topically, locally or systemically. Systemic application includes any method of introducing the compound into the tissues of the body, e.g., intrathecal, epidural, intramuscular, transdermal, intravenous, intraperitoneal, subcutaneous, sublingual, rectal, and oral administration. The specific dosage of antimicrobial to be administered, as well as the duration of treatment, may be adjusted as needed.

The compounds of the invention may be used for the treatment or prevention of infectious disorders caused by a variety of bacterial organisms. Examples include Gram positive and Gram negative aerobic and anaerobic bacteria, including Staphylococci, for example S. aureus; Enterococci, for example E. faecalis; Streptococci, for example S. pneumoniae; Haemophilus, for example H. influenza; Moraxella, for example M. catarrhalis; and Escherichia, for example E. coli. Other examples include Mycobacteria, for example M. tuberculosis; intercellular microbes, for example Chlamydia and Rickettsiae; and Mycoplasma, for example M. pneumoniae.

The ability of a compound of the invention to inhibit bacterial growth, demonstrate in vivo activity, and enhanced pharmacokinetics are demonstrated using pharmacological models that are well known to the art, for example, using models such as the tests described below.

Test A—Antibacterial Assays

The compounds of the present invention were tested against an assortment of Gram-negative and Gram-positive organisms using standard microtitration techniques (Cohen et. al., Antimicrob., 1985;28:766; Heifetz, et. al., Antimicrob., 1974;6:124). The results of the evaluation are shown in Tables 1A and B. In the tables, “—” means no data.

TABLE 1A

| | Minimum Inhibitory | | |
|---|---|---|---|
| | Concentrations μg/mL | | |
| | Gram Negative Bacteria | | |
| | H. influenzae | M. catarrhalis | E. coli |
| Compound | HI-3542 | BC-3531 | MC4100 |
| 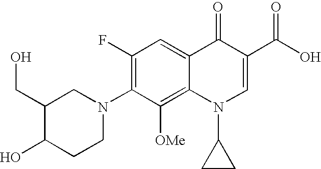 | 0.015 | 0.125 | 0.25 |
| 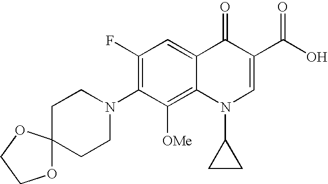 | 0.06 | 0.06 | 0.5 |
| 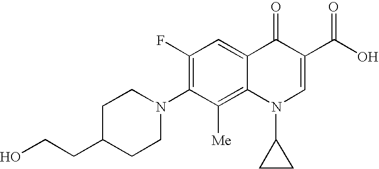 | 0.03 | 0.015 | 0.06 |
| 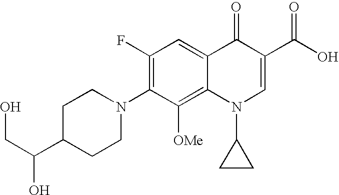 | 0.03 | 0.06 | 0.125 |
| 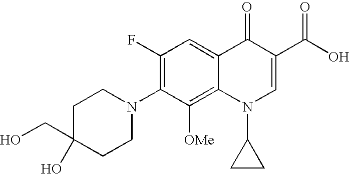 | 0.015 | 0.125 | 0.25 |
| 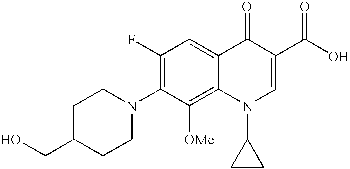 | 0.015 | 0.015 | 0.125 |
| 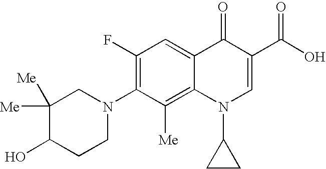 | 0.125 | 0.5 | 2 |
| 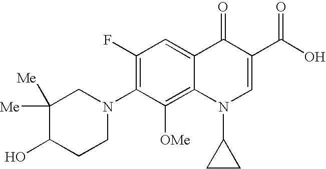 | 0.125 | 0.125 | 0.25 |
| 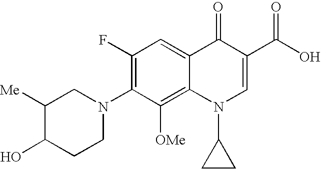 | 0.03 | 0.03 | 0.25 |
| | Minimum Inhibitory | | |
| | Concentrations μg/mL | | |
| | Gram Negative Bacteria | | |
| | H. influenzae | | |
| Compound | HI-3542 | Compound | |
| 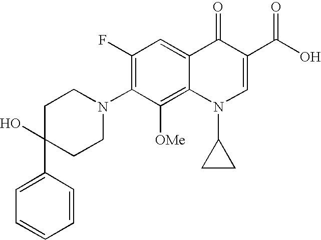 | 2 | 1 | 32 |
| 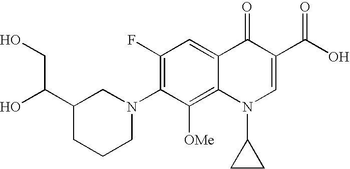 | 0.125 | 0.25 | 1 |
| 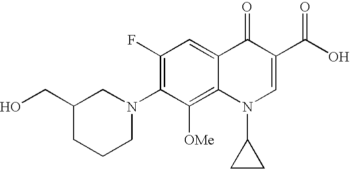 | 0.03 | 0.06 | 0.25 |
| 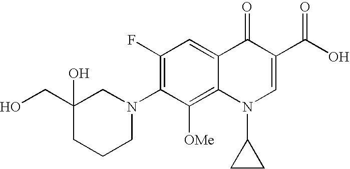 | 0.03 | 0.5 | 1 |
| 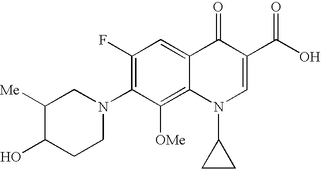 | 0.03 | 0.03 | 0.25 |
| 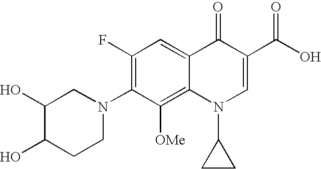 | 0.008 | 0.125 | 0.25 |
| 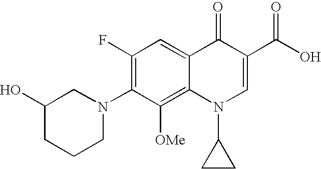 | 0.002 | 0.03 | 0.125 |
| 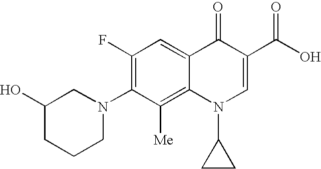 | 0.015 | 0.03 | 0.125 |
| 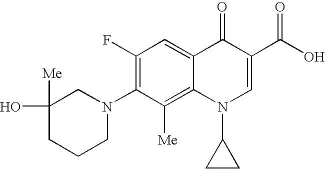 | 0.06 | 0.125 | 0.5 |

TABLE 1B

| | Minimum Inhibitory | | |
|---|---|---|---|
| | Concentrations μg/mL | | |
| | Gram Positive Bacteria | | |
| | E. faecalis | S. aureus | S pyogenes |
| Compound | MGH-2 | UC-76 | C203 |
| 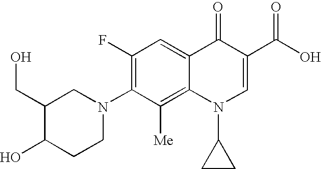 | 0.25 | 0.015 | 0.25 |
| 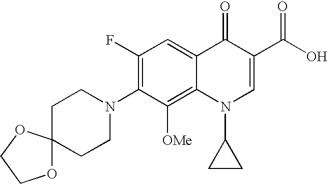 | 0.25 | 0.015 | 0.25 |
| 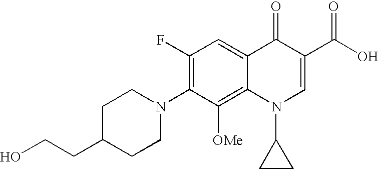 | 0.125 | 0.015 | 0.25 |
| 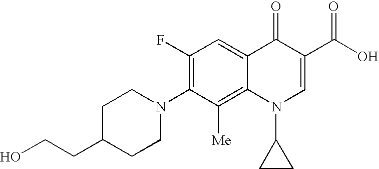 | 0.03 | 0.004 | 0.06 |
| 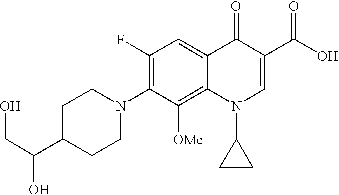 | 0.25 | 0.015 | 0.25 |
| 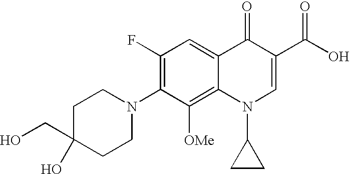 | 1 | 0.015 | 1 |
| 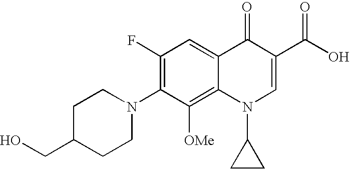 | 0.125 | 0.015 | 0.125 |
| 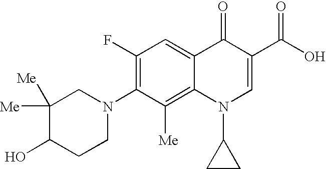 | 8 | 0.25 | 8 |
| 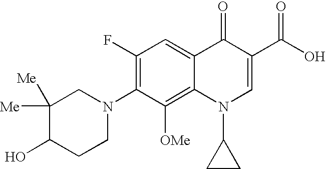 | 0.25 | 0.015 | 0.5 |

| | Minimum Inhibitory | | |
|---|---|---|---|
| | Concentration μg/mL | | |
| | Gram Negative Bacteria | | |
| | H. influenzae | | |
| Compound | HI-3542 | Compound | |
| 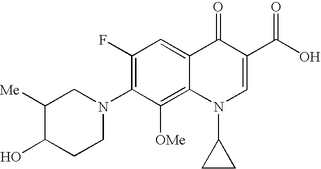 | 0.125 | 0.008 | 0.25 |
| 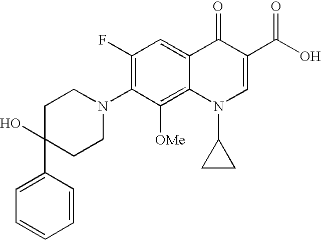 | 32 | 0.25 | 2 |
| 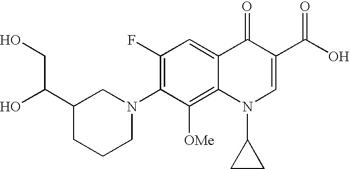 | 8 | 1 | 2 |
| 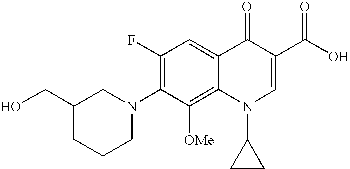 | 0.125 | 0.015 | 0.25 |
| 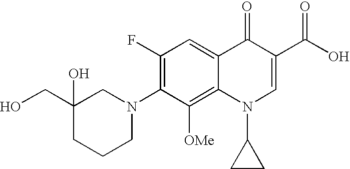 | 1 | 0.06 | 2 |
| 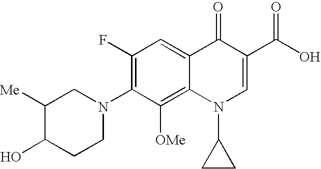 | 0.125 | 0.0008 | 0.25 |
| 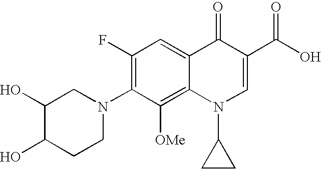 | 0.125 | 0.008 | 0.125 |
| 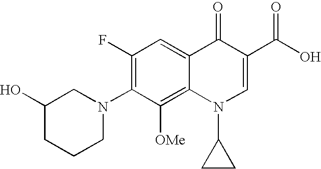 | 0.002 | 0.03 | 0.125 |
| 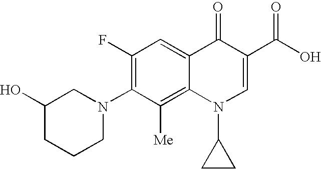 | 0.125 | 0.015 | 0.125 |
| 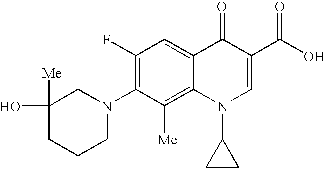 | 0.5 | — | 0.03 |

The following examples are provided to illustrate but not limit the claimed invention.

A. Sidechain Preparation

### EXAMPLE 1

### Preparation of Piperidin-3-ol

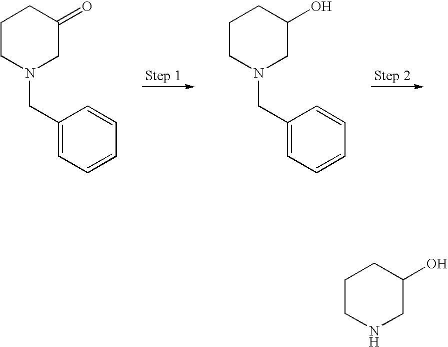 Step (1)

N-benzyl-3-piperidone Hydrochloride was converted to the free base by addition to aqueous K₂CO₃ followed by extraction into ethyl acetate. To a solution of N-benzyl-3-piperidone (2.19 g, 11.57 mmol) in ethanol was added sodium borohydride (NaBH₄) (0.438 g, 11.57 mmol) slowly over ten minutes. The solution was allowed to stir at room temperature overnight, and then concentrated in vacuo. The residue was dissolved in 1.0 N HCl and washed twice with diethylether, then the aqueous solution was basified with 3.0 N KOH to a pH of 12, and then extracted 3 times with dichloromethane. The organic extract was then dried over Na₂SO₄, and concentrated in vacuo. The crude was determined to be 100% pure (1.900 g, 86%) by lc/ms (EI): m/z 192.3 (M+1).

Step (2)

N-benzyl-3-piperidinol (1.90 g, 9.93 mmol) was dissolved in THF:MeOH 1:1, with 20% Pd/C (0.5 g) and subjected to 50 psi of hydrogen gas for 16 hours. The crude product was then filtered through celite and then concentrated in vacuo. The crude product was determined to be pure (0.985 g, 98%) by ¹HNMR (400 MHz, CDCl₃): δ 1.36-1.56 (m, 2H), 1.68-1.81 (m, 2H), 2.60-2.76 (m, 3H), 2.92 (dd, 1H, J=2.7, 11.9 Hz), 3.67 (sept, 1H, J=3.3 Hz).

### EXAMPLE 2

### Preparation of 4-Hydroxymethyl-piperidin-3-ol

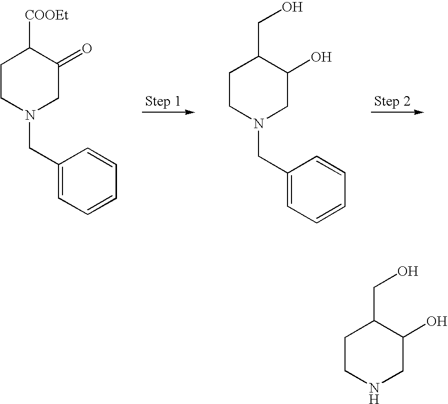 Step (1)

NaBH₄ (5.21 g, 138 mmol) was added in small portions to a stirred mixture of NaOH (0.459 g, 11.55 mmol) and the ester ketone (3.42 g, 11.50 mmol) in anhydrous methanol (50 mL) at room temperature The addition was continued over 1 hour. After stirring for 24 hours, water (60 mL) was added dropwise over 30 minutes and stirring was continued for 24 hours. The methanol was removed in vacuo, and the remaining aqueous residue was extracted 3 times with chloroform, dried (Na₂SO₄), filtered, and concentrated in vacuo. The crude was determined to be clean by lc/ms (EI): m/z 222.3 (M+1).

Step (2)

N-benzyl-4-hydroxymethylpiperidin-3-ol (1.02 g, 4.61 mmol) was dissolved in 50 mL THF:MeOH 1:1, with 20% Pd/C (0.4 g) and subjected to 50 psi of hydrogen gas for 50 hours. The crude product was then filtered through celite and then concentrated in vacuo. The crude product was determined to be pure (0.500 g, 83%) by MS (EI): m/z 131.9 (M+1).

### EXAMPLE 3

### Preparation of 3-Methyl-piperidin-3-ol

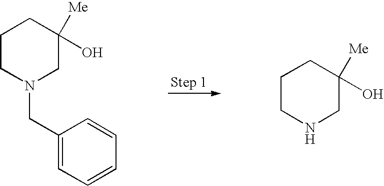 Step (1)

N-benzyl-3-methylpiperidin-3-ol (2.00 g, 9.74 mmol) was dissolved in 50 mL THF:MeOH 1:1, with 20% Pd/C (0.5 g) and subjected to 50 psi of hydrogen gas for 20 hours. The crude product was then filtered through celite and then concentrated in vacuo. The crude product was determined to be pure (0.904 g, 812%) by ¹HNMR (400 MHz, CDCl₃): δ 1.12 (s, 3H), 1.32 (dt, 1H, J=4.9, 13.0 Hz), 1.42-1.49 (m, 1H), 1.58-1.77 (m, 3H), 2.41-2.49 (m, 2H), 2.65-2.71 (m, 1H), 2.89-2.95 (m, 1H).

### EXAMPLE 4

### Preparation of 3-Hydroxymethyl-piperidin-3-ol

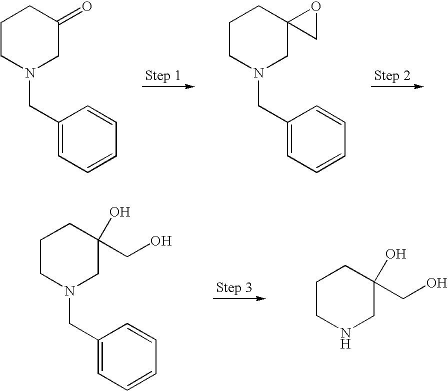 Step (1)

To a solution of trimethylsulfoxonium iodide (4.13 g, 18.78 mmol) in anhydrous DMSO (60 mL) was added NaH (0.450 g, 18.75 mmol) under nitrogen. The solution was stirred until bubbling had stopped and then a solution of the piperidone in DMSO was added slowly with stirring. The resulting mixture was stirred at room temperature for six hours, and then at 50° C. for 40 minutes. The mixture was then cooled to room temperature and quenched with water (12 mL), then extracted with ether (5 times). The combined extracts were washed with water, dried over Na₂SO₄, and then concentrated. The crude was purified by chromatography (gradient: 1:9 EtOAc:Hex to 6:4 EtOAc:Hex) to yield 3.20 g (74%) of the title compound. MS (EI): m/z 204.0 (M+1).

Step (2)

The epoxide (3.62 g, 17.81 mmol) was dissolved in sulfuric acid (200 mL, 0.2 M) and stirred at room temperature for 16 hours. The solution was then basified (to pH=12) with Na₂CO₃, then extracted with ether 5 times. The organic extract was dried over Na₂SO₄, concentrated and purified by chromatography (gradient: 1% EtOH in EtOAc to 10% EtOH in EtOAc over 1200 mL). Thin layer chromatography. visualized by stain: Ammonium Molybdate/Ceric ammonium sulfate/sulfuric acid. 2.02 g of the title compound was collected (51%).LC/MS (EI): m/z 222.3 (M+1).

Step (3)

N-benzyl-3-hydroxymethylpiperidin-3-ol (2.02 g, 9.13 mmol) was dissolved in 50 mL THF:MeOH 1:1, with 20% Pd/C (0.5 g) and subjected to 50 psi of hydrogen gas for 20 hours. The crude product was then filtered through celite and then concentrated in vacuo. The crude product was determined to be pure (1.19 g, 99%) by MS (EI): m/z 132.0 (M+1).

### EXAMPLE 5

### Preparation of 3-Methyl-piperidin-4-ol and 3,3-Dimethyl-piperidin-4-ol

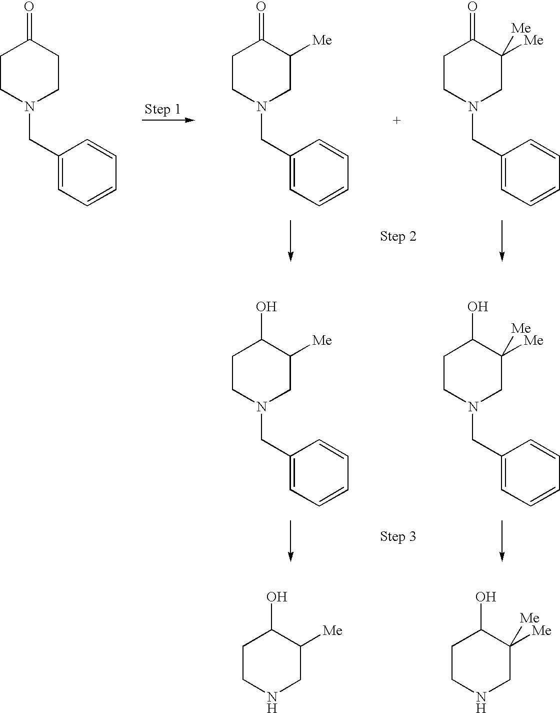 Step (1)

Sodium hydride (4.20 g, 175.0 mmol) was suspended in THF (200 mL), and a solution of the piperidone (18.9 g, 99.9 mmol) and methyl iodide (17.9 g, 126.1 mmol) in THF (20 mL) was added dropwise at room temperature over 5 minutes. The mixture was then heated to 60° C. and stirred for 5 hours. The reaction was then filtered and the filtrate concentrated. The concentrate was poured into 150 mL of water and extracted with 120 mL portions of EtOAc three times. The extract was washed with brine, dried, and concentrated. The crude product was purified by column chromatography (gradient: 12% EtOAc in hexanes to 50% EtOAc in hexanes over 1200 mL). Both mono and di methylation occurred. Separation of both isomers by chromatography was quite facile. 3.53 g of the dimethylated product (16%) was obtained. MS (EI): m/z 218.1 (M+1). 5.91 g (29%) of the monomethylated product was obtained. MS (EI): m/z 204.0 (M+1).

Step (2)

N-benzyl-3-methylpiperidine-4-one (5.82 g, 28.6 mmol) was dissolved in 100 mL of methanol and cooled in an ice bath and NaBH₄ (2.275 g, 60.12 mmol) was added slowly. The reaction was stirred at room temperature overnight, and the remaining NaBH₄ was destroyed by the addition of water. The methanol was then removed under vacuum. The residue was partitioned between water and ethyl acetate, and the aqueous layer re-extracted twice. The combined organic fractions were dried (Na₂SO₄), concentrated to deliver 5.16 g (88%) of the title compound in pure form. MS (EI): m/z 206.1 (M+1).

Step (3)

N-benzyl-3-methylpiperidin-4-ol (5.16 g, 25.13 mmol) was dissolved in 50 mL THF:MeOH 1:1, with 20% Pd/C (0.5 g) and subjected to 50 psi of hydrogen gas for 20 hours. The crude product was then filtered through celite and then concentrated in vacuo. The crude product was analyzed (2.89 g, 99%) by MS (EI): m/z 116.0 (M+1).

### EXAMPLE 6

### Preparation of 1-Piperidin-3-yl-ethane-1,2-diol

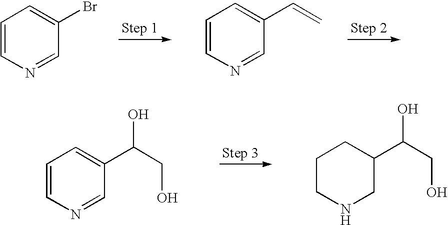 Step (1)

To a solution of 3-bromopyridine (2.49 g, 15.77 mmol) in DMF (50 mL) was added Vinyltributyltin (5.00 g, 15.77 mmol), tetraethylammonium chloride (2.61 g, 15.77 mmol), and dichlorobistriphenylphosphine palladium (0.332 g, 0.473 mmol), and the solution was heated to 70° C. for 3 days. The reaction was then diluted with water, and extracted into EtOAc 3 times. The organic extract was then dried and concentrated, and the crude product purified by chromatography (gradient 9:1 hexanes:EtOAc to 4:6 hexanes:EtOAc) to yield 1.026 g (62%) of the title compound. ¹HNMR (400 MHz, CDCl₃): δ 5.35 (dd, 1H, J=2.2, 11.0 Hz), 5.79 (d, 1H, J=17.6 Hz), 6.66 (dd, 1H, J=11.0, 17.8 Hz), 7.23 (m, 1H), 7.70 (d, 1H, J=7.6 Hz), 8.45 (d, 1H, J=4.9 Hz), 8.59 (s, 1H).

Step (2)

A solution of ADmix alpha (4.83 g, 6.20 mmol) in t-BuOH:H₂O 1:1 (50 mL) was poured into 3-vinylpyridine (0.36 g, 3.42 mmol) at 0° C., and the solution was allowed to warm to room temperature. The reaction was covered with aluminum foil to exclude light and then stirred at room temperature for two days. The mixture was then cooled to 0° C. and sodium sulfite (5.503 g) was added. The mixture was then stirred at room temperature for 1 hour. EtOAc (100 mL) was added, and the layers were separated. The aqueous layer was then extracted with EtOAc twice more and CH₂Cl₂:MeOH 10:1 once. The combined organic layers were then dried, and concentrated. The product (0.330 g, 69%) was pure by lc/ms (EI): m/z 140.3 (M+1).

Step (3)

A mixture of 1-pyridin-3-yl-ethane-1,2-diol (1.03 g, 7.40 mmol), PtO₂ (0.038 g, 0.167 mmol), and 0.85 mL of 1.0 N HCl, in 10 mL of EtOH was subjected to 62 psi of H₂ at room temperature for 6 hours. The mixture was filtered, and the filtrate was concentrated in vacuo to deliver 1.31 g of 1-piperidine-3-yl-ethane-1,2-diol hydrochloride (98%). MS (EI): m/z 146.1 (M+1).

### EXAMPLE 7

### Preparation of (5-Hydroxymethyl-piperidin-3-yl)-methanol

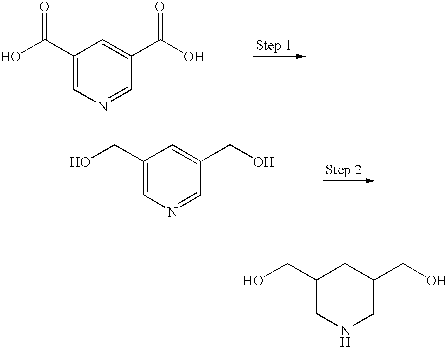 Step (1)

To 3,5-Pyridinedicarboxylic acid (1.00 g, 5.98 mmol) in dry THF (5 mL) at 0° C. was added borane THF complex (1 M, 30.0 mL, 30.0 mmol) slowly. The solution was then brought to room temperature and stirred overnight. The reaction was then quenched with 10% HCl, extracted with EtOAc, washed with water, and dried over Na₂SO₄. The solvent was removed in vacuo and the crude material was purified by flash chromatography (gradient: EtOAc to 9:1 EtOAc:EtOH over 1200 mL) to yield 520 mg of the title compound as a white solid. LC/MS (EI): m/z 140.3 (M+1).

Step (2)

A mixture of 3,5-dihydroxymethylpyridine (500 mg, 3.59 mmol) PtO₂ (18.8 mg, 0.0826 mmol), and 0.85 mL of 1 N HCl, in 10 mL of EtOH was subjected to 62 psi of H₂ at room temperature for 6 hours. The mixture was filtered, and the filtrate was concentrated in vacuo to deliver 3,5-dihydroxymethylpiperidine hydrochloride.

### EXAMPLE 8

### Preparation of 4-Hydroxymethyl-piperidin-4-ol

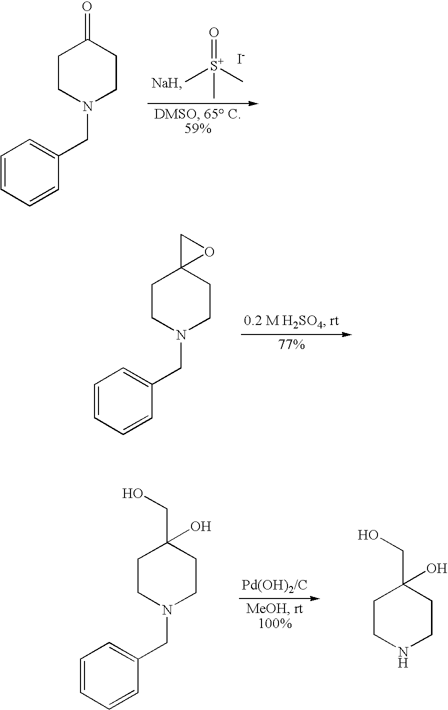 Step (1)

DMSO (10 mL) was added to sodium hydride (60% dispersion, 321 mg, 13.4 mmol) to deliver a gray suspension. To this suspension was added trimethylsulfoxonium iodide (2.95 g, 13.4 mmol) in several portions causing gas evolution. After complete addition, the milky mixture was stirred at room temperature for 20 minutes, whereupon n-benzyl-4-piperidone in DMSO (10 mL) was added to deliver a light orange solution. This solution was then heated at 65° C. for 3.5 hours. The solution was cooled to room temperature, and then slowly poured into water (50 mL) cooled in an ice bath. The aqueous layer was then extracted with ethyl acetate (3×200 mL). The combined organic layers were then washed with water (2×100 mL), dried (magnesium sulfate), filtered, and concentrated under reduced pressure. The resulting residue was purified via flash chromatography eluting with dichloromethane:methanol (20:1) to deliver the title compound as a yellow oil (Yield: 1.33 g, 59%). MS (APCI) (m+1/z) 204.0.

Step (2)

A solution of the epoxide (1.33 g, 6.54 mmol) in aqueous 0.2 M sulfuric acid (82 mL) was stirred at room temperature for 20 hours. The solution was cooled to 0° C., and sodium carbonate was added until the pH of the aqueous mixture reached 12. The mixture was then diluted with water (20 mL), and extracted with ethyl acetate (3×200 mL). The combined organic layers were dried (magnesium sulfate), filtered, and concentrated under reduced pressure. The resulting white solid was purified via flash chromatography eluting with a gradient of dichloromethane:methanol (5:1 to 1:1) to deliver the title compound as a waxy solid (1.12 g, 77%). MS (APCI) (m+1/z) 221.1.

Step (3)

A mixture of the benzylamine (1.05 g, 4.75 mmol) and 20% Pd(OH)₂/C (0.15 g) in methanol (50 mL) was hydrogenated at 50 psi for 3 hours. The mixture was filtered, and the filtrate was concentrated under reduced pressure to deliver the title compound as a white solid (Yield 593 mg, 95%). MS (APCI) (m+1/z)=132.0

### EXAMPLE 9

### Preparation of 3-Hydroxymethyl-piperidin-4-ol

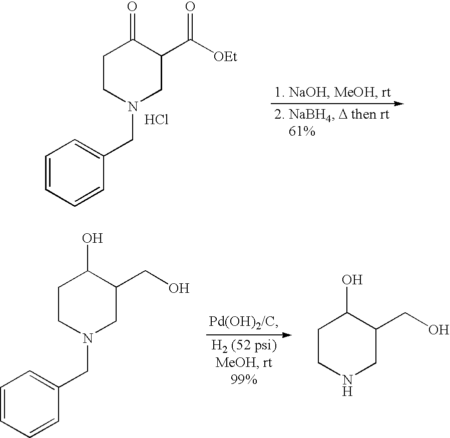

The title compound was prepared as provided in Synthesis 1999, 1937-1943.

### EXAMPLE 10

### Preparation of 1-Piperidin-4-yl-ethane-1,2-diol Hydrochloride Salt

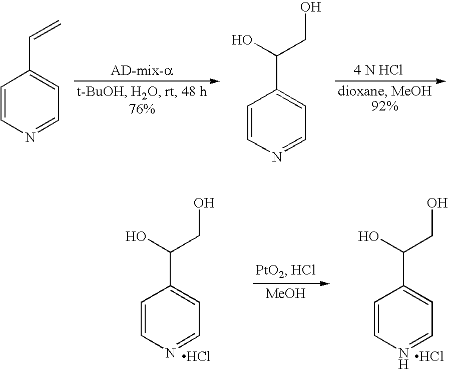

The title compound was prepared as provided in Tetrahedron: Asymm. 1995, 6, 505-518 (Reference for Sharpless asymmetric dihydroxylation); J. Org. Chem. 1965, 30, 1331-1333; 1969 U.S. Pat. No. 3,464,997 (Reference for hydrogenation).

### EXAMPLE 11

### Preparation of 1-Piperidin-4-yl-propane-1,3-diol

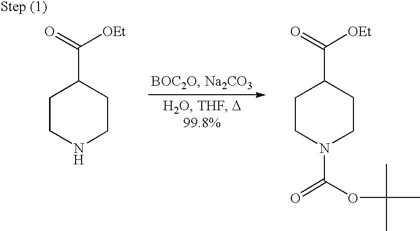

A mixture of ethyl isonipecotate (5.00 g, 31.8 mmol), di-tert-butyl dicarbonate (7.64 g, 34.99 mmol), and sodium carbonate (6.74 g, 63.6 mmol) in water/tetrahydrofuran (50 mL: 20 ml) was heated at reflux for 2 hours. The mixture was allowed to cool to room temperature, and was extracted with ethyl acetate (2×200 mL). The combined organic layers were dried (magnesium sulfate), filtered, and concentrated under reduced pressure. A thick yellow oil was afforded, 8.17 g (99.8%). MS (APCI) (m+1)/z 258, (M-BOC+1)/z, 157.9.

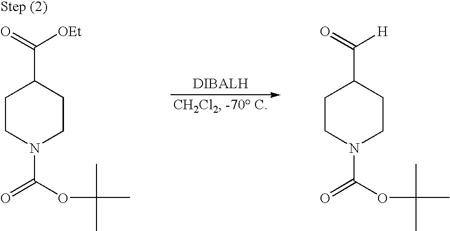

To a solution of the ethyl ester (2.44 g, 9.48 mmol) in dichloromethane (60 mL) at −70° C. diisobutylaluminum hydride (1.0 M in dichloromethane, 11.38 mL) was added dropwise over 1 hour so that the internal temperature never rose above −70° C. The solution was stirred at −70° C. for 4 hours. An additional volume of diisobutylaluminum hydride (1.0 M, 4.74 mL) was added. After an additional 2 hours at −70° C., methanol (2 mL) was added followed by saturated aqueous sodium bicarbonate (20 mL). Diethylether (200 mL) was added, and the mixture was then allowed to reach room temperature, where it was stirred for 10 minutes. The suspension was then filtered through a pad of celite. The filtrate was then washed with water (100 mL). The layers were separated, and the organic layer was dried (magnesium sulfate), filtered, and concentrated under reduced pressure. A clear viscous oil was afforded, 1.82 g.

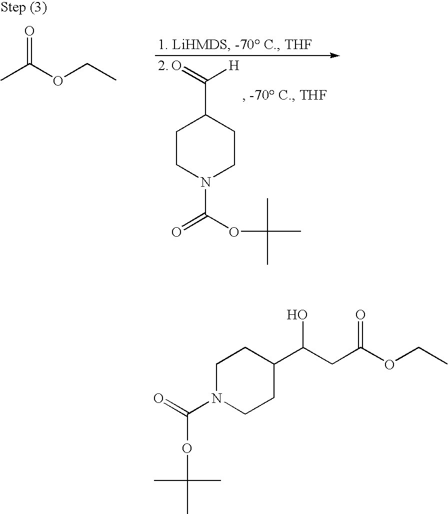

A solution of lithium hexamethyldisilazide (1.0 M in tetrahydrofuran, 10.9 mL) was added dropwise to a solution of ethyl acetate (804 mg, 9.12 mmol) in tetrahydrofuran (80 mL) at −70° C. After complete addition of lithium hexamethyldisilazide, the resulting solution was stirred at −70° C. for 2 hours, then the aldehyde in tetrahydrofuran (5 mL) was added. The reaction mixture was then stirred at −70° C. for 4 hours; saturated aqueous ammonium chloride (15 mL) was added, and the mixture was allowed to reach room temperature. The biphasic system was then partitioned between diethyl ether (250 mL) and brine (40 mL). The layers were separated, and the aqueous layer was extracted with diethyl ether (2×2001 mL). The combined organic layers were dried (magnesium sulfate), filtered, and concentrated under reduced pressure. The oil was purified via flash chromatography eluting with a gradient of hexanes:ethyl acetate (3:1 to 1:1) to deliver 1.01 g (44%) of the title compound as a clear oil. MS (APCI) (M+1)/z 302.1, (M-BOC+1)/z) 202.1.

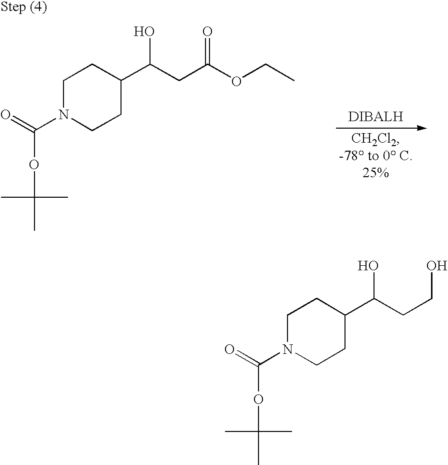

A solution of diisobutylaluminum hydride (1.0 M in dichloromethane, 16.04 mL) was added to a solution of the β-hydroxyethyl ester (967 mg, 3.21 mmol) in dichloromethane (20 mL) at −70° C. over several minutes. The solution was then stirred at 0° C. for 2 hours. The solution was cooled to −50° C. and saturated aqueous sodium bicarbonate was added. The mixture was allowed to reach room temperature whereupon it was diluted with diethyl ether (100 mL) and then swirled for 2-3 minutes. The mixture was then filtered through a pad of celite. The filtrate was then washed with water (2×40 mL), and the layers were separated. The aqueous layer was then extracted with ethyl acetate (2×50 mL). The combined organic layers were washed with saturated aqueous sodium chloride (30 mL), dried (magnesium sulfate), filtered, and concentrated under reduced pressure. A thick clear oil was afforded, 369 mg (25%). MS (APCI) (M+1)/z 260.2, (M-BOC+1)/z 160.1

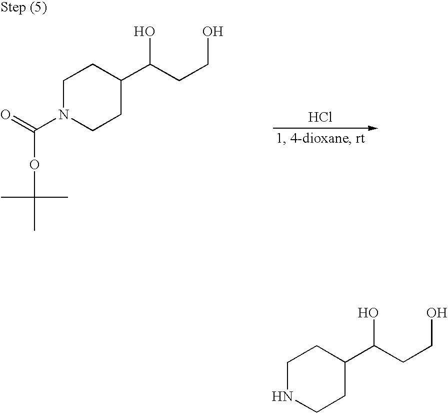

A solution of hydrogen chloride in dioxane (4.0M, 2.01 mL) was added to a solution of the carbamate (209 mg, 0.806 mmol) in methanol (500 μL). The resulting solution was stirred at room temperature for 2 hours. The solvent was then removed under reduced pressure to give a semi-solid. The semi-solid was then dissolved in a mixture of methanol and tetrahydrofuran (1:1, 4 mL), and ˜300 mg of Amberlite® IRA-400 hydroxide resin was added. The suspension was stirred for 45 min, and then filtered through a glass wool plug. A yellow oil was afforded, 127 mg (99%). MS (APCI) (M+1)/z 160.0.

### EXAMPLE 12

### Preparation of (R) and (S) 1-Piperidin-4-yl-ethane-1,2-diol

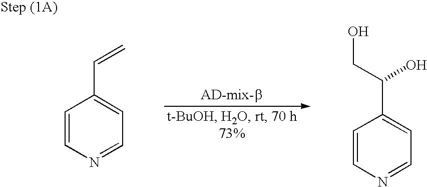

Vinylpyridine (1.07 g, 10.2 mmol) was added to a suspension of AD-mix-β (14.35 g) in a mixture of tert-butanol:water (1:1, 90 mL) at room temperature for 70 hours. The reaction mixture was then cooled to 0° C., and sodium sulfite (16.34 g) was added. The mixture was then stirred at room temperature for 1 hour. Ethyl acetate (200 ml) was added, and the layers were separated. The aqueous layer was then extracted with ethyl acetate (2×200 mL). The combined organic layers were then dried (magnesium sulfate), filtered, and concentrated under reduced pressure to deliver a viscous clear oil. The oil was purified via medium pressure liquid chromatography (MPLC) eluting with a gradient of acetone and hexanes (5:1 to 15:1) to deliver 1.03 g (73%) of the title compound as a white solid: optical rotation=−17.8 in ethanol at 589 nm, c=20 mg/mL; m.p. 56-59° C. MS (APCI) (M+1)/z 139.9, (M+CH₃CN+1)/z 180.9.

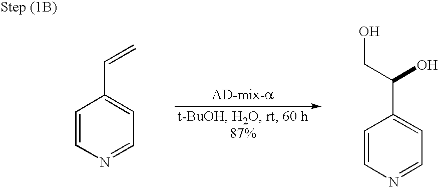

A mixture of vinylpyridine (1.25 g, 11.9 mmol) and AD-mix-α (16.76 g) in tert-butanol:water (1:1, 100 mL) was stirred at room temperature for 60 hours. The reaction mixture was then cooled to 0° C., and sodium sulfite (18.32 g) was added. The mixture was then stirred at room temperature for 1 hour. Ethyl acetate (200 ml) was added, and the layers were separated. The aqueous layer was then extracted with ethyl acetate (2×200 mL). The combined organic layers were then dried (magnesium sulfate), filtered, and concentrated under reduced pressure to deliver a viscous clear oil. The oil was purified via flash chromatography with a gradient of acetone and hexanes (5:1 to 15:1) to deliver 1.44 g (87%) of the title compound as a white solid: optical rotation =+15.8 in ethanol at 589 nm, c=23 mg/mL; m.p.=58-61° C.; MS (APCI) (m+1/z) 139.9, (M+CH₃CN+1/z) 180.9.

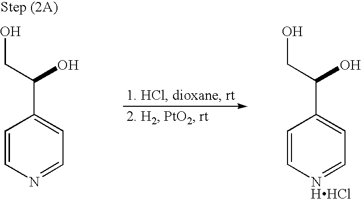

A solution of 4 N HCl in dioxane (5.06 mL) was added to a solution of the pyridine (1.41 g, 10.1 mmol) in MeOH (5 mL). The solution was stirred at room temperature for 1 hour, and solvent was removed under reduced pressure to give a white solid, 1.68 g (94%). A mixture of the HCl salt of the pyridine (1.57 g, 8.94 mmol), platinum oxide (100 mg, 0.44 mmol), 1.0 N hydrochloric acid (1.4 mL) in ethanol (20 mL) was kept at 55 psi of hydrogen gas at room temperature for 7.5 hours. The solvent was then removed under reduced pressure to deliver 1.67 g of a yellow oil (composition of sample: 80% title compound, 20% mono-ol based on ¹H NMR). MS (APCI) (M+1)/z 145.9.
Step (1B)

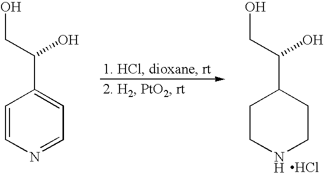

The title compound was prepared as described as above. MS (APCI) (M+1)/z 145.9.

### EXAMPLE 13

### Preparation of (4-Hydroxymethyl-piperidin-4-yl)-methanol

Step (1) Piperidine-1,4,4-tricarboxylic acid-1-tert butyl ester-4,4-diethyl Ester

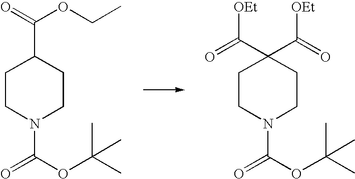

A solution of Piperidine-1,4-dicarboxylic acid-1-tertbutyl ester-4-ethyl ester (5.00 g) in tetrahydrofuran (50 mL) was added to a cooled (−78° C.) solution of lithium diisopropylamide in tetrahydrofuran (28.66 mL, 2 M), and the resulting solution was stirred at −78° C. for 2 hours then at 40° C. for 3 hours. The solution was cooled back to −78° C., and a solution of ethyl chloroformate (6.50 mL) in tetrahydrofuran (40 mL) was added, and the reaction was allowed to warm to room temperature over 16 hours. Ammonium chloride (aqueous), hydrochloric acid, and ethyl acetate were added, and the layers were separated. The aqueous portion was re-extracted with ethyl acetate (3 times), and the combined organic extracts were dried, and concentrated. The crude material was reacted further without purification.
Step (2) 4,4-Bis-hydroxymethyl-piperidine-1-carboxylic Acid Tert-Butyl Ester

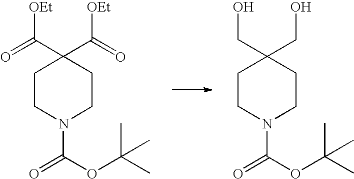

Crude Piperidine-1,4,4-tricarboxylic acid-1-tert butyl ester-4,4-diethyl ester (19.43 mmol) was dissolved in toluene/tetrahydrofuran (1:1, 200 mL) and lithium borohydride (2.247 g) was added. The reaction was warmed to 60° C. for 16 hours, then cooled, then ammonium chloride (aqueous) was added slowly until the organic layer was a clear solution. The mixture was adjusted to pH=12 with sodium carbonate (aqueous), and the layers were separated. The aqueous layer was extracted with ethyl acetate (2 times), and the combined organic extracts were dried, and concentrated. The product was purified by chromatography (gradient: 1:1 ethyl acetate:hexanes to pure ethyl acetate). The desired compound was obtained in pure form (1.02 g, 21% for two reactions).
Step (3) (4-Hydroxymethyl-piperidin-4-yl)-methanol

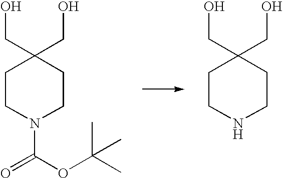

To a solution of 4,4-Bis-hydroxymethyl-piperidine-1-carboxylic acid tert-butyl ester (1.02 g) in dichloromethane (20 mL) was added trifluoroacetic acid (10 mL), and the reaction was allowed to sit for an hour at room temperature. The solvent was then removed in vacuo, and the residue was azeotroped with methanol. The amine was freebased by stirring in hydroxy resin/methanol/tetrahydrofuran for 30 minutes. The slurry was filtered and the filtrate concentrated to yield 4,4-Bis-hydroxymethyl piperidine as an oil (0.6 g, 99% yield).

### Coupling Reactions

### EXAMPLE 14

### Preparation of 1-Cyclopropyl-6-fluoro-7-(4-hydroxy-3,3-dimethyl-piperidin-1-yl)-8-ethoxy-4-oxo-1,4-dihydro-quinoline-3-carboxylic Acid

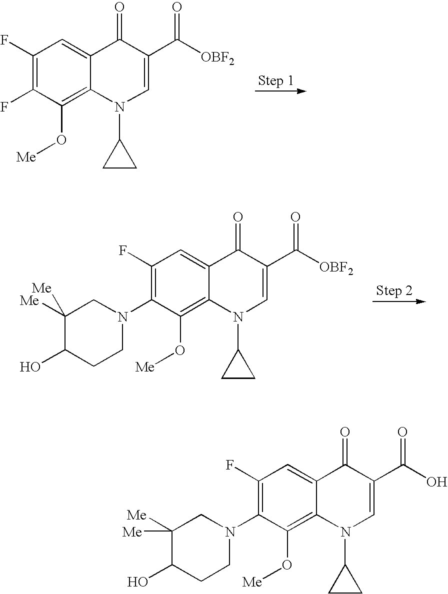 Step (1)

1-cyclopropyl-6,7-difluoro-8-methoxy-4-oxo-1,4-dihydro-quinoline-3-carboxylic acid difluoroboronate ester (400 mg, 1.17 mmol), 4-hydroxy-3,3-dimethylpiperidine (Example 5, 301 mg, 2.33 mmol), and triethylamine (0.325 mL, 2.33 mmol) were heated to reflux in 10 mL of dry acetonitrile overnight under a nitrogen atmosphere.

Step (2)

The crude mixture from step (1) was concentrated, and then re-dissolved in EtOH (10 mL) with additional triethylamine (0.813 mL, 5.83 mmol). The solution was heated to reflux for 5-6 hours. The mixture was then cooled to R.T., concentrated, and purified by recrystallization from MeOH to deliver 220 mg (47% for two steps) of the title compound as a yellow solid. MS (IE): m/z 405.1 (M+1).

### EXAMPLE 15

### Preparation of 1-Cyclopropyl-6-fluoro-7-(4-hydroxy-4-hydroxymethyl-piperidin-1-yl)-8-methoxy-4-oxo-1,4-dihydro-quinoline-3-carboxylic Acid

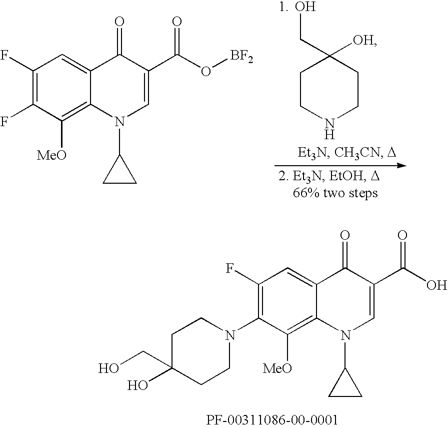

Prepared as provided in Example 14, except 4-hydroxymethyl-piperidin-4-ol (Example 8) was used in the coupling reaction. Yield: 236 mg, 66% for two steps. MS (APCI) (m+1)/z 407.1; m.p. 200-210 (decomposition).

### EXAMPLE 16

### Preparation of 1-Cyclopropyl-6-fluoro-7-(4-hydroxy-3-hydroxymethyl-piperidin-1-yl)-8-methoxy-4-oxo-1,4-dihydro-quinoline-3-carboxylic Acid

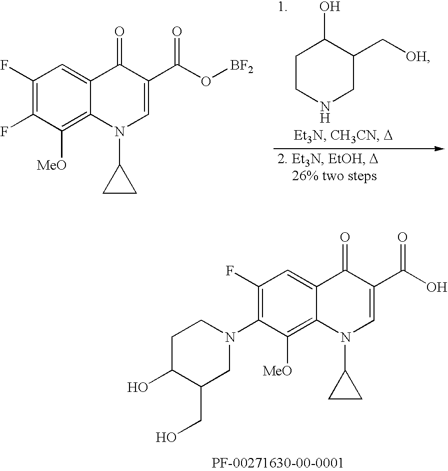

Prepared as provided in Example 14, except 3-Hydroxymethyl-piperidin-4-ol (Example 9) was used in the coupling reaction. Yield: 93 mg, 26% for two steps. MS (APCI) (m+1/z) 407.1; m.p.=115-125° C.

### EXAMPLE 17

### Preparation of 1-Cyclopropyl-7-[4-(1,2-dihydroxy-ethyl)-piperidin-1-yl]-6-fluoro-8-methoxy-4-oxo-1,4-dihydro-quinoline-3-carboxylic Acid

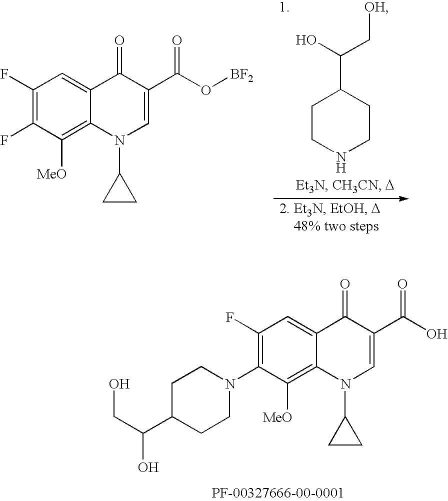

Prepared as provided in Example 14, except 1-Piperidin-4-yl-ethane-1,2-diol hydrochloride salt (Example 10) was used in the coupling reaction. Yield: 178 mg, 48% for two steps). MS (APCI) m+1/z 421.1; m.p.=200-204° C.

### EXAMPLE 18

### Preparation of 1-Cyclopropyl-7-[4-(1,3-dihydroxy-propyl)-piperidin-1-yl]-6-fluoro-8-methoxy-4-oxo-1,4-dihydro-quinoline-3-carboxylic Acid

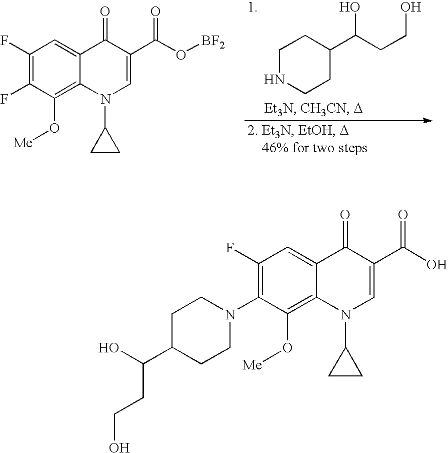

The title compound was prepared as described in Example 14 using the 6,7-diflouro-8-methoxyquinolone (160 mg, 0.466 mmol), the diol (126 mg, 0.791 mmol), and triethylamine (325 μL, 2.33 mmol) for step one and triethylamine (650 μL, 4.67 mmol) for step two. The crude solid was then partially dissolved in dichloromethane (30 mL). Hexanes (25 mL) were then slowly added to cause solid formation. The mixture was then placed in the refrigerator for 1 h. The suspension was filtered to deliver a yellow solid, 93 mg (46%). m.p.=210-214 C.; MS (APCI) (m+1)/z 435.2.

### EXAMPLE 19

### Preparation of 1-Cyclopropyl-7-[4-(1S,2-dihydroxy-ethyl)-piperidin-1-yl]-6-fluoro-8-methyl-4-oxo-1,4-dihydro-quinoline-3-carboxylic Acid

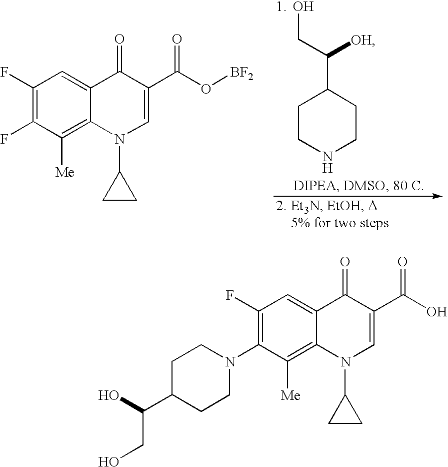

A solution of the 6,7-diflouro-8-methylquinolone (717 mg, 2.19 mmol), the diol (684 mg, 4.71 mmol), and diisopropylethylamine (2.67 mL, 15.4 mmol) in dimethylsulfoxide was heated at 80 C for 3 days. The solution was concentrated to remove excess diisopropyl-ethylamine. The resulting solution was diluted with ethanol (10 mL) and then treated with triethylamine (3.05 mL, 21.9 mmol). The solution was then heated in a 95° C. oil bath for 5 hours. The solvent was removed under reduced pressure to deliver a brown solution. The solution was diluted with dichloromethane (200 mL) and washed with 1N aqueous hydrochloric acid (20 mL). The organic layer was then washed with water (2×30 mL), dried (magnesium sulfate), filtered, and then concentrated under reduced pressure to give a brown solid. The crude product was then purified via reverse phase preparatory high performance liquid chromatography (HPLC) eluting with a gradient of 90% water (0.1% formic acid)/10% acetonitrile (0.1% formic acid) to 10% water (0.1% formic acid)/90% acetonitrile (0.1% formic acid) to deliver 43 mg (5%) of the title compound as a yellow solid. m.p. 220-222° C.; MS (APCI) (m+1)/z 405.1.

### EXAMPLE 20

### Preparation of 1-Cyclopropyl-7-[4-(1R,2-dihydroxy-ethyl)-piperidin-1-yl]-6-fluoro-8-methoxy-4-oxo-1,4-dihydro-quinoline-3-carboxylic Acid

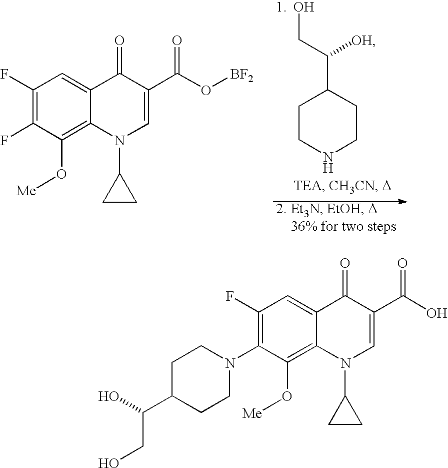

The title compound was prepared as described in Example 14 using the 6,7-diflouro-8-methoxyquinolone (239 mg, 0.697 mmol), the diol (202 mg, 1.39 mmol), and triethylamine (680 μL, 4.89 mmol) for step one and triethylamine (970 μL, 6.96 mmol) for step two. The crude product was dissolved in dichloromethane, and hexanes (25 mL) were then slowly added to cause solid formation. The mixture was then placed in the refrigerator for 1 h. The suspension was filtered to deliver a yellow solid, 104 mg (36%). m.p.=198-202 C.; MS (APCI) (m+1)/z 421.1.

### EXAMPLE 21

### Preparation of 1-Cyclopropyl-7-[4-(1R,2-dihydroxy-ethyl)-piperidin-1-yl]-6-fluoro-8-difluromethoxy-4-oxo-1,4-dihydro-quinoline-3-carboxylic Acid

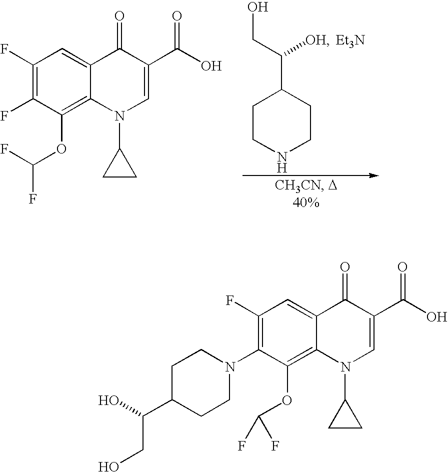

A white suspension of the fluoroquinolone (189 mg, 0.571 mmol), the diol (167 mg, 1.15 mmol), and triethylamine (557 mL, 4.0 mmol) in acetonitrile (5 mL) was heated in a 85° C. oil bath for 16 hours, whereupon a solution was afforded. The solution was then concentrated under reduced pressure. The residue was then partitioned between dichloromethane (100 mL) and 1 N aqueous hydrochloric acid (15 mL). The layers were separated and the aqueous layer was extracted with with dichloromethane (2×25 mL). The combined organic layers were dried (magnesium sulfate), filtered, and concentrated under reduced pressure a yellow solid was afforded, 177 mg. The crude product was purified via reverse phase medium pressure liquid chromatography (MPLC) eluting with a gradient of water/acetonitrile (95:5 to 25:75) to deliver 103 mg (40%) of the title compound as a yellow solid. m.p. 228-230° C.; MS (APCI) (m+1)/z 457.1.

### EXAMPLE 22

### Preparation of 1-Cyclopropyl-6-fluoro-7-[4-(2-hydroxy-ethyl)-piperidin-1-yl]-8-methyl-4-oxo-1,4-dihydro-quinoline-3-carboxylic Acid

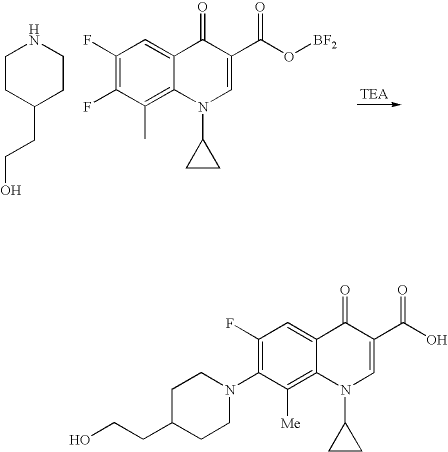

1-Cyclopropyl-6,7-difluoro-8-methyl-4-oxo-1,4-dihydro-quinoline-3-carboxylic acid boronate ester (400 mg) was dissolved in dimethylsulfoxide (3 mL), with 2-Piperidine-4-yl-ethanol (316 mg), and diisopropylethylamine, and heated to 80° C. for two days. The solution was then diluted with ethanol, and heated to 80° C. for another four hours, then concentrated, and purified by reverse-phase medium pressure liquid chromatography MPLC (95:5 water:acetonitrile to 40:60 water:acetonitrile over 600 mL) to yield 160 mg (34%) of 1-Cyclopropyl-6-fluoro-7-[4-(2-hydroxy-ethyl)-piperidin-1-yl]-8-methyl-4-oxo-1,4-dihydro-quinoline-3-carboxylic acid as well as 40 mg of the 6-coupled isomer. LC/MS Rₜ=2.93 min, m/z=389.2. isomer LC/MS Rₜ=2.68 min, m/z=389.2.

### EXAMPLE 23

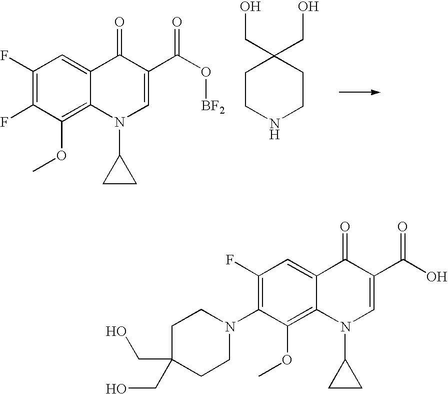

1-Cyclopropyl-6,7-difluoro-8-methoxy-4-oxo-1,4-dihydro-quinoline-3-carboxylic acid boronate ester (300 mg), 4,4-Bis-hydroxymethyl piperidine (254 mg), and triethylamine (0.61 mL) were heated to reflux in acetonitrile (10 mL) overnight under a nitrogen atmosphere. The crude mixture was then concentrated, and then re-dissolved in ethanol (10 mL) and triethylamine (0.61 mL). The solution was heated to reflux for 5-6 hours. The mixture was then concentrated, and purified by reverse-phase chromatography (gradient: 95:5 water:acetonitrile to 40:60 water:acetonitrile with 0.5% formic acid buffer). 1-Cyclopropyl-6-fluoro-7-[4,4-(bishydroxymethyl)-piperidin-1-yl]-8-methoxy-4-oxo-1,4-dihydro-quinoline-3-carboxylic acid eluted and was obtained as a bright yellow solid (240 mg, 65% yield). LC/MS Rₜ=2.14 min, m/z=421.2.

### EXAMPLE 24

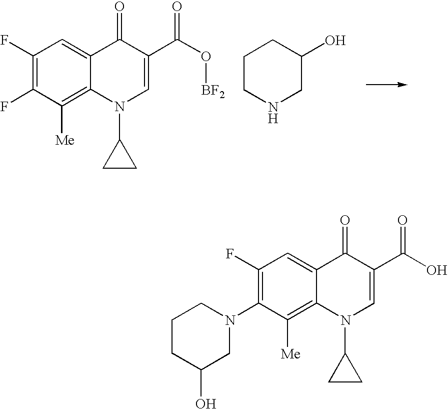

1-Cyclopropyl-6,7-difluoro-8-methyl-4-oxo-1,4-dihydro-quinoline-3-carboxylic acid boronate ester (300 mg), was dissolved in dimethylsulfoxide (3 mL) with diispropylethal amine (0.799 mL) and 3-hydroxypiperidine (232 mg), and heated to 80° C. for three days. The solution was then diluted with ethanol (5 mL), and heated to 80° C. for another four hours, then concentrated. The dimethylsulfoxide solution that remained was loaded directly onto a prep-hplc column for purification. 1-Cyclopropyl-6-fluoro-7-[3-hydroxy-piperidin-1-yl]-8-methyl-4-oxo-1,4-dihydro-quinoline-3-carboxylic acid eluted and was obtained as a bright yellow solid (24 mg, 7% yield). LC/MS Rₜ=2.62 min, m/z=361.1.

### EXAMPLE 25

### Preparation of 1-Cyclopropyl-6-fluoro-7-(3-hydroxy-4-hydroxymethyl-piperidin-1-yl)-8-methyl-4-oxo-1,4-dihydro-quinoline-3-carboxylic Acid

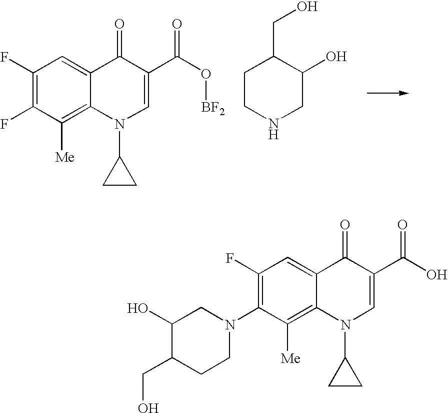

1-Cyclopropyl-6,7-difluoro-8-methyl-4-oxo-1,4-dihydro-quinoline-3-carboxylic acid boronate ester (300 mg) was dissolved in dimethylsulfoxide (3 mL) with 3-hydroxy-4-hydroxymethyl-piperidine (241 mg) and diisopropylethyl amine (0.799 mL), and heated to 80° C. for three days. The solution was then diluted with ethanol, and heated to 80° C. for another four hours, then concentrated, and the crude dimethylsulfoxide solution was purified by reverse-phase MPLC. Two regioisomers which eluted together were then separated by prep-hplc. 1-Cyclopropyl-6-fluoro-7-[3-hydroxy-4-hydroxymethyl-piperidin-1-yl]-8-methyl-4-oxo-1,4-dihydro-quinoline-3-carboxylic acid eluted and was obtained as a bright yellow solid (29 mg, 7% yield). LC/MS Rₜ=1.64 min, m/z=391.2.

### EXAMPLE 26

### Preparation of 7-(4,4-Bis-hydroxymethyl-piperidin-1-yl)-1-cyclopropyl-6-fluoro-8-methyl-4-oxo-1,4-dihydro-quinoline-3-carboxylic Acid

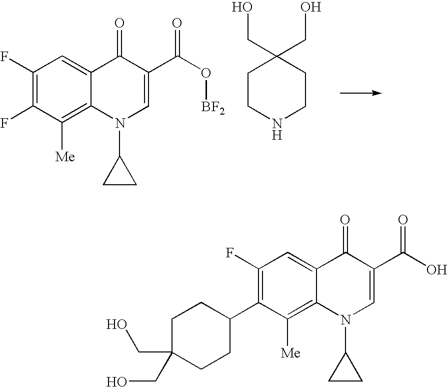

1-Cyclopropyl-6,7-difluoro-8-methyl-4-oxo-1,4-dihydro-quinoline-3-carboxylic acid boronate ester (600 mg) was dissolved in dimethylsulfoxide with 4,4-Bis hydroxymethyl piperidine (533 mg) and diisopropylethyl amine (1.6 mL), and heated to 80° C. for three days. The solution was then diluted with ethanol, and heated to 80° C. for another four hours, then concentrated, and purified by reverse-phase MPLC (95:5 water:acetonitrile to 40:60 water:acetonitrile over 600 mL). Two regioisomers eluted separately, both in pure form: 1-Cyclopropyl-6-fluoro-7-[4,4-bis hydroxymethyl-piperidin-1-yl]-8-methyl-4-oxo-1,4-dihydro-quinoline-3-carboxylic acid was obtained as a bright yellow solid (33 mg, 4% yield), LC/MS Rₜ=2.08 min, m/z=405.2. 1-Cyclopropyl-7-fluoro-6-[4,4-bis hydroxymethyl-piperidin-1-yl]-8-methyl-4-oxo-1,4-dihydro-quinoline-3-carboxylic acid was obtained as a bright yellow solid (70 mg, 8% yield), LC/MS Rₜ=1.81 min, m/z=405.2.

### EXAMPLE 27

### General Procedure for Coupling Sidechain and Quinazolinedione

A mixture of the piperidine (2 equiv), core (1 equiv) and 1,1,3,3-tetramethylguanidine (3 equiv) in dimethylsulfoxide (0.5-1 mMol) is heated at 85-100° C. for 12-36 hours. The solution is poured into saturated aqueous ammonium chloride and extracted with chloroform. The combined organic layers are dried with magnesium sulfate and concentrated in vacuo. The product is purified on a silica gel column eluting with 0 to 10% methanol in dichloromethane to give the coupled product.

### EXAMPLE 28

### The following illustrates representative pharmaceutical dosage forms, containing a compound of Formula I (“Invention Compound”), for therapeutic or prophylactic use in humans

| (i) Tablet | mg/tablet |
|---|---|
| ‘Invention Compound’ | 25.0 |
| Lactose | 50.0 |
| Corn Starch (for mix) | 10.0 |
| Corn Starch (paste) | 10.0 |
| Magnesium Stearate (1%) | 3.0 |
| | 300.0 |

The invention compound, lactose, and corn starch (for mix) are blended to uniformity. The corn starch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of pathogenic bacterial infections.

| (ii) Tablet | mg/capsule |
|---|---|
| ‘Invention Compound | 10.0 |
| Colloidal Silicon Dioxide | 1.5 |
| Lactose | 465.5 |
| Pregelatinized Starch | 120.0 |
| Magnesium Stearate (1%) | 3.0 |
| | 600.0 |

| | |
|---|---|
| (iii) Preparation for | |
| Oral Solution | Amount |

| | | |
|---|---|---|
| ‘Invention Compound’ | 400 | mg |
| Sorbitol Solution (70% N.F.) | 40 | mL |
| Sodium Benzoate | 20 | mg |
| Saccharin | 5 | mg |
| Cherry Flavor | 20 | mg |
| Distilled Water q.s. | 100 | mL |

The sorbitol solution is added to 40 mL of distilled water, and the invention compound is dissolved therein. The saccharin, sodium benzoate, flavor, and dye are added and dissolved. The volume is adjusted to 100 mL with distilled water. Each milliliter of syrup contains 4 mg of invention compound.

(iv) Parenteral Solution

In a solution of 700 mL of propylene glycol and 200 mL of water for injection is suspended 20 g of an invention compound. After suspension is complete, the pH is adjusted to 6.5 with 1 N hydrochloric acid, and the volume is made up to 1000 mL with water for injection. The Formulation is sterilized, filled into 5.0 mL ampoules each containing 2.0 mL, and sealed under nitrogen.

| (v) Injection 1 (1 mg/mL) | Amount |
|---|---|
| ‘Invention Compound’ | 1.0 |
| Dibasic Sodium Phosphate | 12.0 |
| Monobasic Sodium Phosphate | 0.7 |
| Sodium Chloride | 4.5 |
| N Sodium hydroxide solution | q.s. |
| (pH adjustment to 7.0-7.5) | |
| Water for injection | q.s. ad 1 mL |

| (vi) Injection 2 (10 mg/mL) | Amount |
|---|---|
| ‘Invention Compound’ | 10.0 |
| Dibasic Sodium Phosphate | 1.1 |
| Monobasic Sodium Phosphate | 0.3 |
| Polyethylene glyco 400 | 200.0 |
| N hydrochloric acid solution | q.s. |
| (pH adjustment to 7.0-7.5) | |
| Water for injection | q.s. ad 1 mL |

| (vii) Injection 2 (10 mg/mL) | Amount |
|---|---|
| ‘Invention Compound’ | 20.0 |
| Oleic Acid | 10.0 |
| Trichloromonofluoromethane | 5,000.0 |
| Dichlorodifluoromethane | 10,000.0 |
| Dichlorotetrafluoroethane | 5,000.0. |

All patents, and patent documents are incorporated by reference herein, as though individually incorporated by reference. The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

1. A compound of formula I 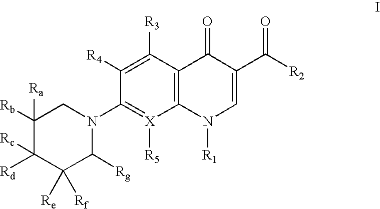
or a pharmaceutically acceptable salt thereof, wherein:
X is N or C, provided that when X is N, R₅ is absent at that position;
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is OH,
OBF₂,
O(C₁-C₆)alkyl,
O(C₃-C₆)cycloalkyl, 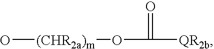
wherein m is an integer of from 1 to 10, Q is O or is absent, and R₂ₐ is H or (C₁-C₆)alkyl and R_{2b} is (C₁-C₆)alkyl, aryl, or heteroaryl,
O—(CHR₂ₐ)ₙ—Y, wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
NR_{2d}, wherein R_{2d} is as defined above, 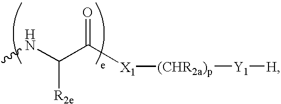
wherein “ [symbol] ” indicates the point of attachment, 2a is as defined above, R₂ₑ is H or (C₁-C₆)alkyl, e is an integer of from 1 to 10, p is an integer of from 2 to 10, and X₁ and Y₁ are each independently NH or O;
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
R₁ and R₅, together with the carbons to which they are attached, form a substituted or unsubstituted 6-membered ring containing an additional heteroatom selected from O, S, NH, or N(C₁-C₆)alkyl;
at least one of Rₐ, R_{b}, R_{c}, R_{d}, Rₑ, R_{f}, and R_{g} is
OH,
OPO(OH)₂,
OPO(O(C₁-C₆)alkyl)₂, 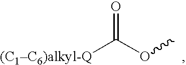
wherein “ 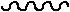
” indicates the point of attachment and Q is O or is absent 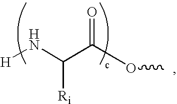
wherein “ 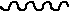
” indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)hydroxyalkyl
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 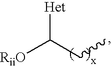
wherein “ 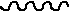
” indicates the point of attachment, het is a 5- or 6-membered heterocyclo or heteroaryl group, and x is an integer of from 0 to 10; 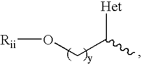
wherein “ 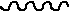
” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, 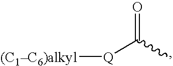
as defined above, or 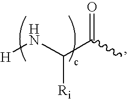
as defined above;
and the others of Rₐ, R_{b}, R_{c}, R_{d}, Rₑ, R_{f}, and R_{g} are each independently H, (C₁-C₆)alkyl,
OH,
OPO(OH)₂,
OPO(O(C₁-C₆)alkyl)₂, 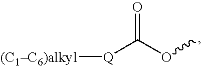
wherein “ 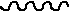
” indicates the point of attachment and Q is O or is absent 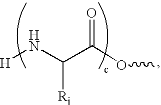
wherein “ 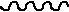
” indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 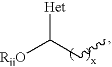
wherein “ 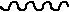
” indicates the point of attachment, het is a 5- or 6-membered heterocyclo or heteroaryl group, and x is an integer of from 0 to 10; 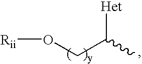
wherein “ 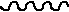
” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, 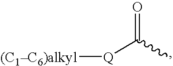
as defined above, or 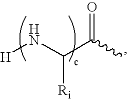
as defined above; or
R_{c} and Rₑ, together with the carbons to which they are attached, form a substituted or unsubstituted 4, 5, or 6-membered ring containing 0, 1, 2, or 3 heteroatoms selected from P, NH, N(C₁-C₆)alkyl, S, or O; or
R_{c} and R_{d} or Rₑ and R_{f}, independently, together with the carbon to which they are attached, form a substituted or unsubstituted 3, 4, 5, or 6-membered ring containing 0, 1, 2, or 3 heteroatoms selected from NH, N(C₁-C₆)alkyl S, or O; and
provided that when R₅ is OMe, and Rₑ is OH, R_{d} is not H or Me.

2. The compound of claim 1, wherein
R₁ is (C₁-C₆)cycloalkyl and halo(C₁-C₆)cycloalkyl, aryl, or heteroaryl;
R₃ is H or NH₂;
R₄ is H or halo; and
R₅ is halo, methyl, trifluoromethyl, methoxy, fluoromethoxy, difluoromethoxy, or trifluoromethoxy.

3. The compound of claim 2, wherein
R₁ is cyclopropyl,
fluorocyclopropyl, 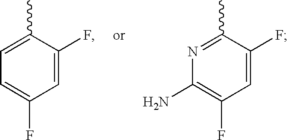
R₃ is H or NH₂;
R₄ is H or F; and
R₅ is halo,
methyl,
trifluoromethyl, or
methoxy.

4. The compound of claim 3, which is: 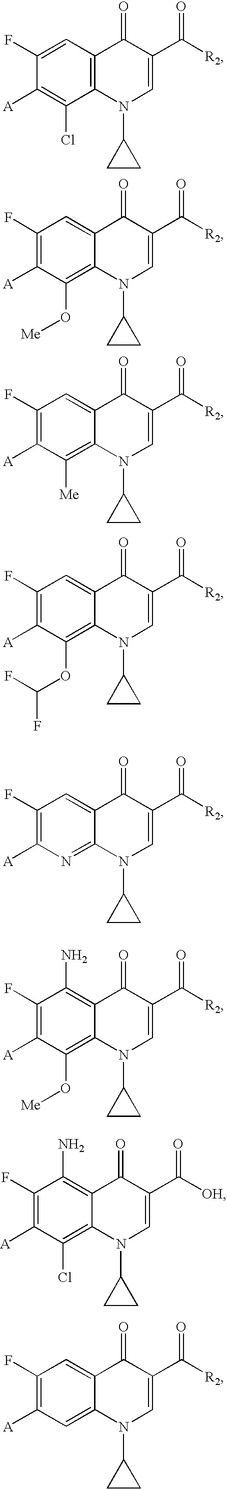 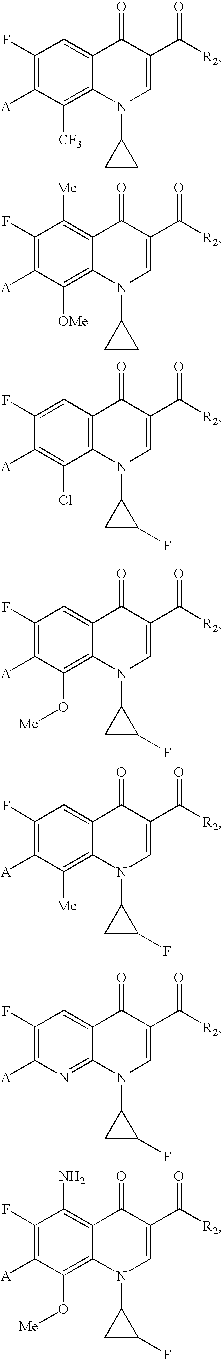 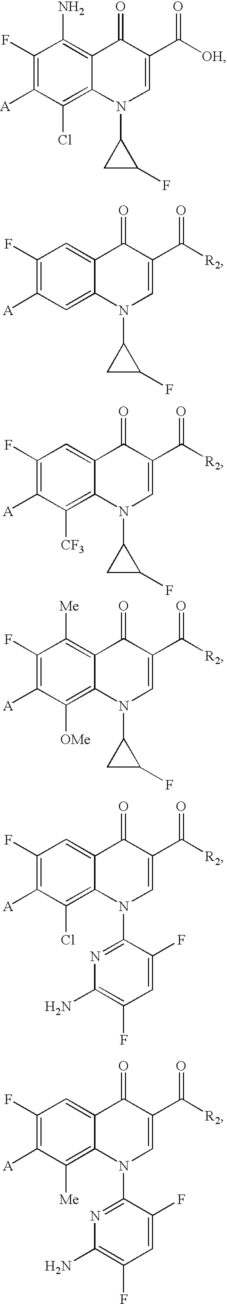 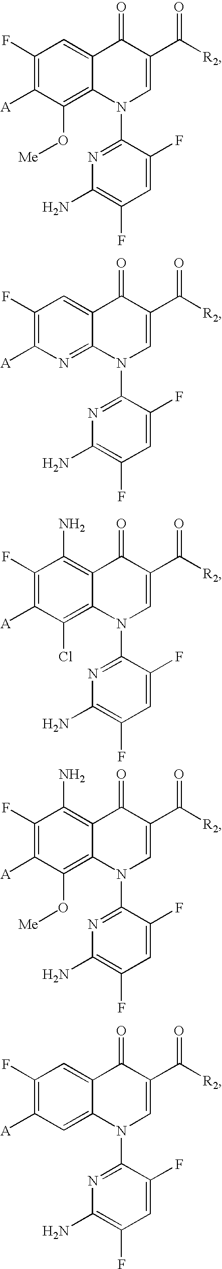 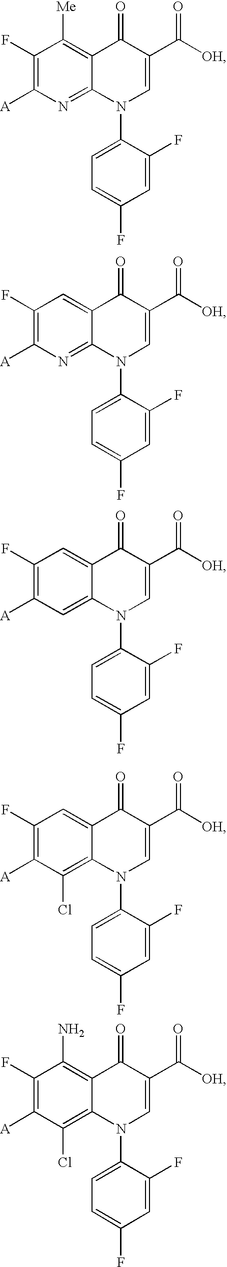 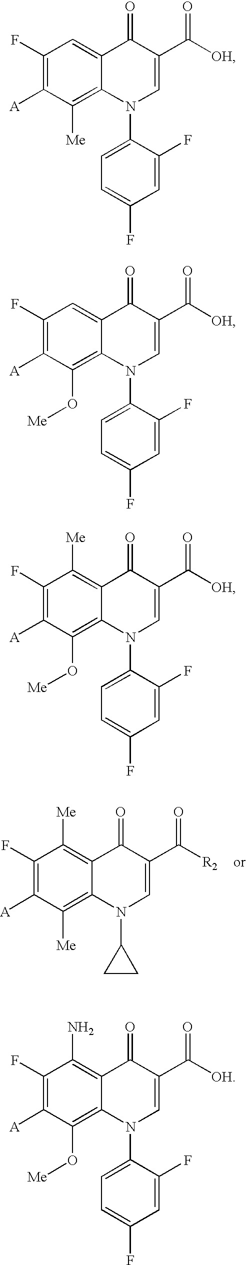
wherein R₂ is OH, O(C₁-C₆)alkyl, or OBF₂, R₄ is H or F, and A is

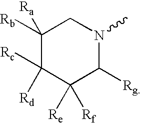

5. The compound of claim 4, wherein R_{g} in A is H.

6. The compound of claim 5, wherein 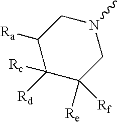
is 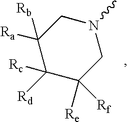
wherein 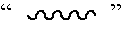
” indicates the point of attachment, and includes

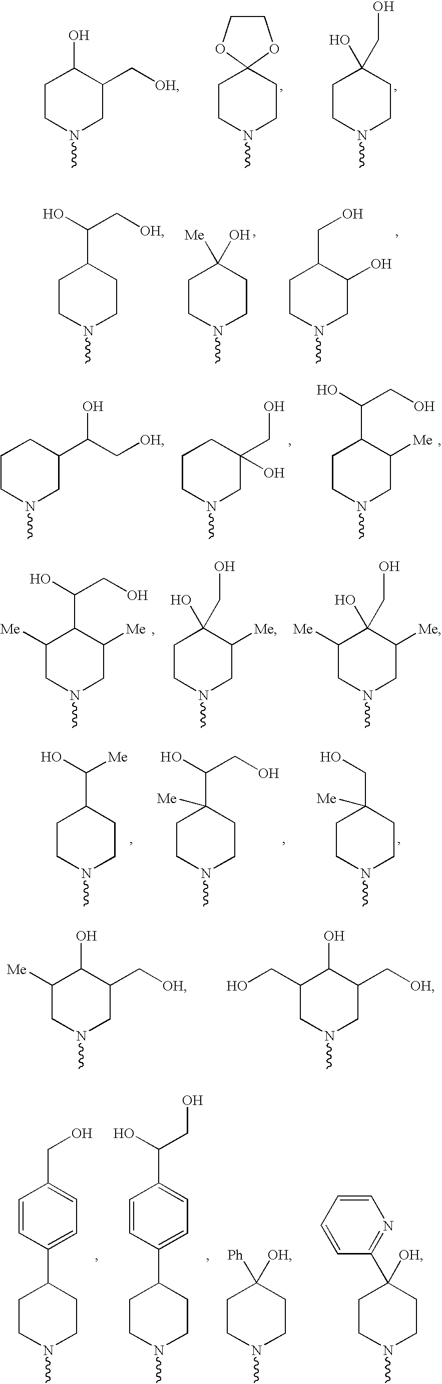

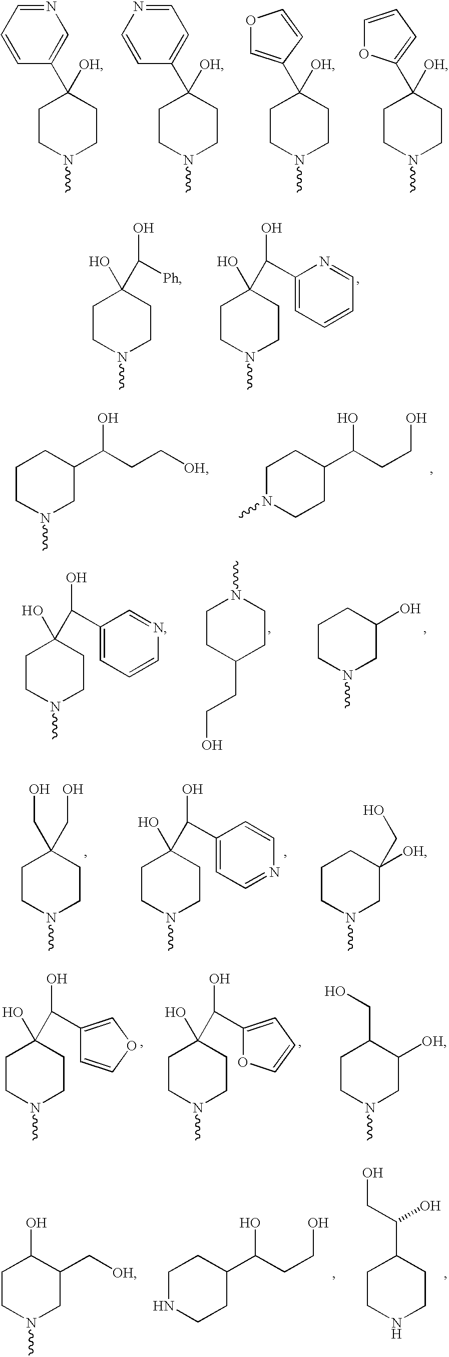

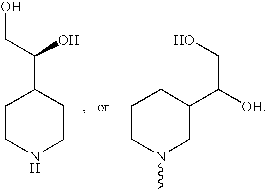

7. A compound of claim 1 having formula II, formula III, formula IV, formula V or formula VI: 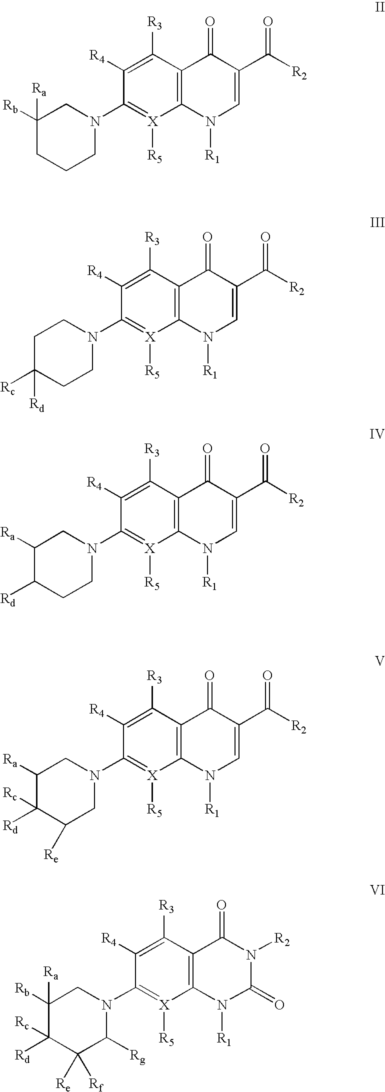
or a pharmaceutically acceptable salt thereof, wherein:
when the compounds has formula II:
X, and R₁-R₅ are as defined for compounds of formula I and
Rₐ is OH,
OPO(OH)₂,
OPO(O(C₁-C₆)alkyl)₂, 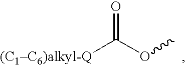
wherein 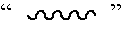
” indicates the point of attachment and Q is O or is absent 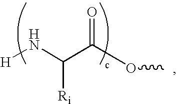
wherein 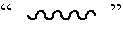
” indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)hydroxyalkyl
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 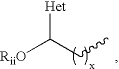
wherein 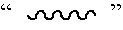
” indicates the point of attachment, het is a 5- or 6-membered heterocyclo or heteroaryl group, and x is an integer of from 0 to 10; 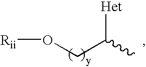
wherein 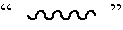
” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, 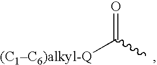
as defined above, or 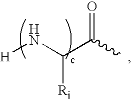
as defined above;
and R_{b} is
H,
(C₁-C₆)alkyl, 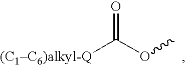
wherein 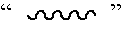
” indicates the point of attachment and Q is O or is absent 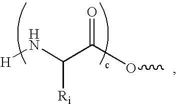
wherein 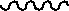
” indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)hydroxyalkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 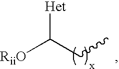
wherein 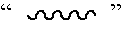
” indicates the point of attachment, het is a 5- or 6-membered heterocyclo or heteroaryl group, and x is an integer of from 0 to 10; 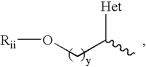
wherein 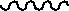
” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, 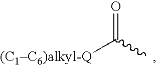
as defined above, or 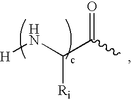
as defined above;
wherein when the compound has formula III:
X, and R₁-R₅ are as defined for compounds of formula I and
R_{c} is OH,
OPO(OH)₂,
OPO(O(C₁-C₆)alkyl)₂, 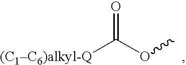
wherein 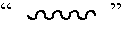
” indicates the point of attachment and Q is O or is absent 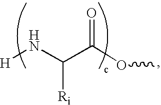
wherein 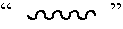
” indicates the point of attachment. Rᵢ is H or (C₁-C₆)alkyl, and c is an integer having a value of from 1 to 10,
RᵢᵢO(C₁-C₆)hydroxyalkyl
RᵢᵢO(C₁-C₆ haloalkyl,
RᵢᵢO(C₃-C₆)cycloalkyl,
RᵢᵢO(C₁-C₆)alkyl-O—,
RᵢᵢO(C₁-C₆ haloalkyl-O—,
RᵢᵢO(C₃-C₆)Cycloalkyl-O—, 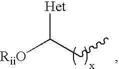
wherein 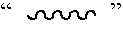
” indicates the point of attachment, het is a 5- or 6-membered heterocyclo or heteroaryl group, and x is an integer of from 0 to 10; 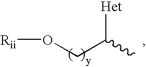
wherein 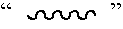
” indicates the point of attachment, het is as defined above, and y is an integer of from 1 to 10,
wherein Rᵢᵢ is H,
(C₁-C₆)alkyl,
P(OH)₂,
PO(O(C₁-C₆)alkyl)₂, 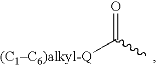
as defined above, or 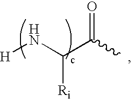
as defined above,
and R_{d} is
H,
(C₁-C₁₆)alkyl, 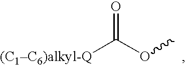
wherein 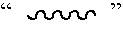
” indicates the point of attachment and Q is O or is absent 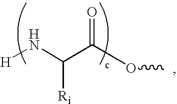
wherein 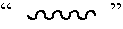
” indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)hydroxyalkyl,
RᵢᵢO(C₁-C₆ haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 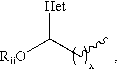
wherein 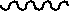
” indicates the point of attachment, het is a 5- or 6-membered heterocyclo or heteroaryl group, and x is an integer of from 0 to 10, 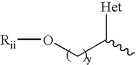
wherein 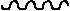
” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, 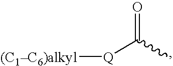
as defined above, or 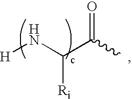
as defined above;
wherein when the compound has formula IV:
X, and R₁-R₅ are as defined for compounds of formula I and
Rₐ is OH,
OPO(OH)₂,
OPO(O(C₁-C₆)alkyl)₂, 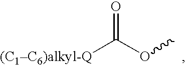
wherein 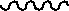
” indicates the point of attachment and Q is O or is absent 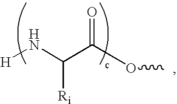
wherein 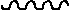
” indicates the point of attachment, Rᵢ is H or (C₁-C₆)alkyl, and c is an integer having a value of from 1 to 10,
RᵢᵢO(C₁-C₆)alkyl)
RᵢᵢO(C₁-C₆)hydroxyalkyl
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 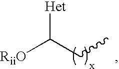
wherein 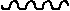
” indicates the point of attachment, het is a 5- or 6-membered heterocyclo or heteroaryl group, and x is an integer of from 0 to 10; 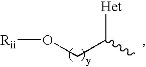
wherein 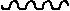
” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, 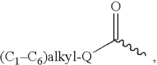
as defined above, or 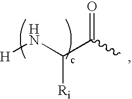
as defined above;
and R_{d} is
(C₁-C₆)alkyl 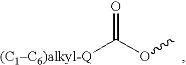
wherein 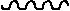
” indicates the point of attachment and is O is O or is absent 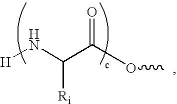
wherein 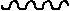
” indicates the point of attachment, Rᵢ is H or (C₁-C₆)alkyl, and c is an integer having a value of from 1 to 10,
RᵢᵢO(C₁-C₆)hydroxyalkyl,
RᵢᵢO(C₁-C₆)haloalkyl,
RᵢᵢO(C₁-C₆)cycloalkyl,
RᵢᵢO(C₃-C₆)alkyl-O—,
RᵢᵢO(C₁-C₆)haloalkyl-O—.
RᵢᵢO(C₃-C₆)cycloalkyl-O—, 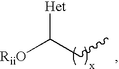
wherein 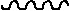
” indicates the point of attachment, het is a 5- or 6-membered heterocyclo or heteroaryl group, and x is an integer of from 0 to 10; 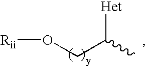
wherein 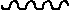
” indicates the point of attachment, het is as defined above, and v is an integer of from 1 to 10;
wherein Rᵢᵢ is H
(C₁₋₆)alkyl,
PO(OH)₂,
PO(O(C₁-C₆)alkyl)₂, 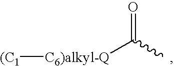
as defined above, or 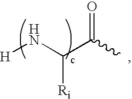
as defined above;
wherein when the compound has formula V:
X, and R₁-R₅ are as defined for compounds of formula I and
Rₐ and Rₑ are each independently (C₁-C₆)alkyl or hydroxy(C₁-C₆)alkyl;
R_{d} is H or OH;
R_{c} is hydroxy(C₁-C₆)alkyl;
provided that when R₅ is OMe, and R_{c} is OH, R_{d} is not H or Me; and
wherein the compound has formula VI:
X is N or C, provided that when X is N, R₅ is absent at that position;
halo(C₁-C₆)alkyl,
(C₃-C₆)Cycloalkyl
halo(C₃-C₆)cycloalkyl
aryl, and
heteroaryl;
R₂ is H,
NH₂, 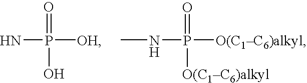
NH(C₁-C₆)alkyl,
NH(C₃-C₆)cycloalkyl,
NH-heteroaryl,
NHSO₂-(C₁-C₆)alkyl,
NHSO₂-aryl,
NHSO₂-heteroaryl, 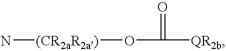
wherein, Q is O or is absent, and R₂ₐ and R_{2a′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 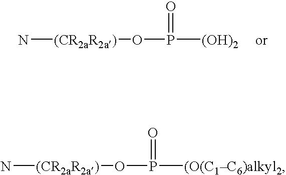
wherein R₂ₐ and R_{2a′} are as defined above, 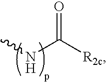
wherein 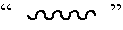
indicates the point of attachment, p is 0 or 1, and
R_{2c} is H
(C₁-C₆)alkyl,
(C₃-C₇)cycloalkyl,
aryl,
heterocyclo,
heteroaryl, or 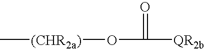
or —(CHR₂ₐ)ₙ—Y, wherein R₂ₐ, R_{2b}, and Q are as defined above, n is an integer from 0 to 10, and Y is OH, OPO(OH)₂, OPO(O(C₁-C₆)alkyl)₂, NR_{2d}R₂ₑ, wherein R_{2d} and R₂ₑ each independently H, (C₁-C₆)alkyl, or (C₃-C₇)cycloalkyl, 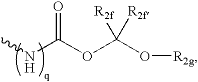
wherein q is 0 or 1R_{2f}, and R_{2f′} are each independently H, (C₁-C₆)alkyl, aryl, or heteroaryl, or taken together with the carbon to which they are attached form a 3, 4, 5, or 6 membered ring, and R_{2g} is
(C₁-C₆)alkyl,
(C₃-C₇)cycloalkyl,
aryl, or
heterocyclo, or
heteroaryl;
R₃, R₄, and R₅ are each independently H,
halo,
NH₂,
(C₁-C₆)alkyl,
halo(C₁-C₆ alkyl,
(C₁-C₆)alkoxy, or
halo(C₁-C₆)alkoxy;
at least one of Rₐ, R_{b}, R_{c}, R_{d}, Rₑ, R_{f} and R_{g} is
OH,
OPO(OH)₂,
OPO(O(C₁-C₆)alkyl)₂, 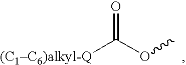
wherein 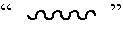
indicates the point of attachment and 0 is O or is absent 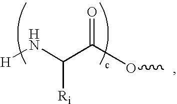
wherein 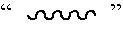
indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)hydroxyalkyl
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 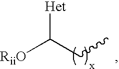
wherein 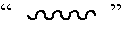
indicates the point of attachment, het is a 5- or 6-membered heterocyclo or heteroaryl group, and x is an integer of from 0 to 10; 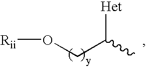
wherein 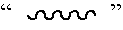
indicates the point of attachment, het is as defined above, and y is an integer of from 1 to 10;
wherein Rᵢᵢ is H,
(C₁-C₆)alkyl,
PO(OH)₂,
PO(O(C₁-C₆alky 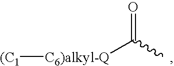
as defined above, or 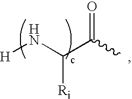
as defined above;
and the others of Rₐ, R_{b}, R_{c}, R_{d}, Rₑ, R_{f}, and R_{g} are each independently H,
H,
(C₁-C₆)alkyl,
OH,
OPO(OH)₂,
OPO(O(C₁-C₆)alkyl)₂, 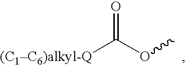
wherein “ 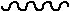
” indicates the point of attachment and Q is O or is absent 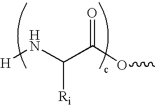
wherein “ 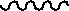
” indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C-4-C₆)cycloalkyl-O—, 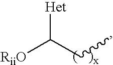
wherein “ 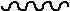
” indicates the point of attachment, het is a 5- or 6-membered heterocyclo or heteroaryl group, and x is an integer of from 0 to 10; 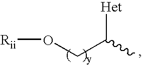
wherein “ 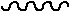
” indicates the point of attachment, het is as defined above, and y is an integer of from 1 to 10;
wherein Rᵢᵢ is H.
(C—C₆)alkyl,
PO(OH)₂,
PO(O(C₁-C₆)alkyl)₂, 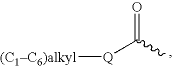
as defined above, or 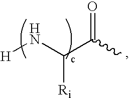
as defined above; or
R_{c} and Rₑ, together with the carbons to which they are attached, form a substituted or unsubstituted 4, 5, or 6-membered ring containing 0, 1, 2, or 3 heteroatoms selected from P, NH, N(C₁-C₆)alkyl, S, or O; or
R_{c} and R_{d} or Rₑ and R_{f}, independently, together with the carbon to which they are attached, form a substituted or unsubstituted 3, 4, 5, or 6-membered ring containing 0, 1, 2, or 3 heteroatoms selected from P, NH, N(C₁-C₆alkyl, S, or O.

8. The compound of claim 7, wherein
R₁ is (C₁-C₆)cycloalkyl and halo(C₁-C₆)cycloalkyl, aryl, or heteroaryl;
R₃ is H or NH₂;
R₄ is H or halo; and
R₅ is halo, methyl, trifluoromethyl, methoxy, fluoromethoxy, difluoromethoxy, or trifluoromethoxy.

9. The compound of claim 7, wherein
R₁ is cyclopropyl,
fluorocyclopropyl, 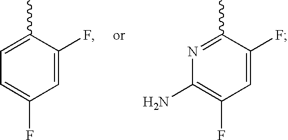
R₃ is H or NH₂;
R₄ is H or F; and
R₅ is halo,
methyl,
trifluoromethyl, or
methoxy.

10. The compound of claim 7, which is: 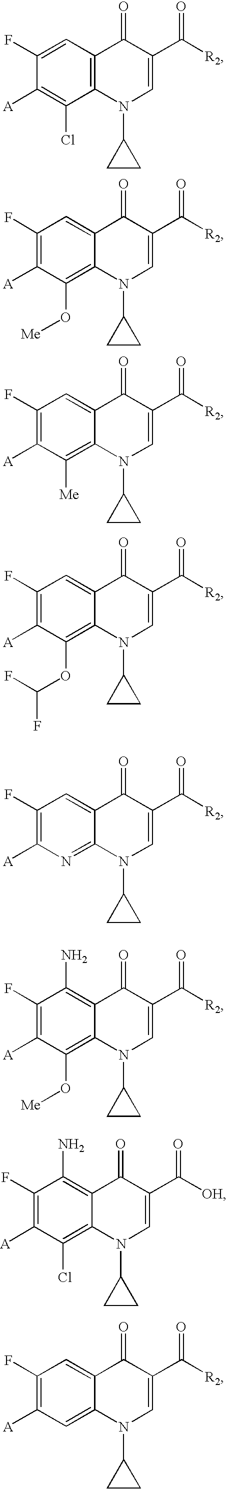 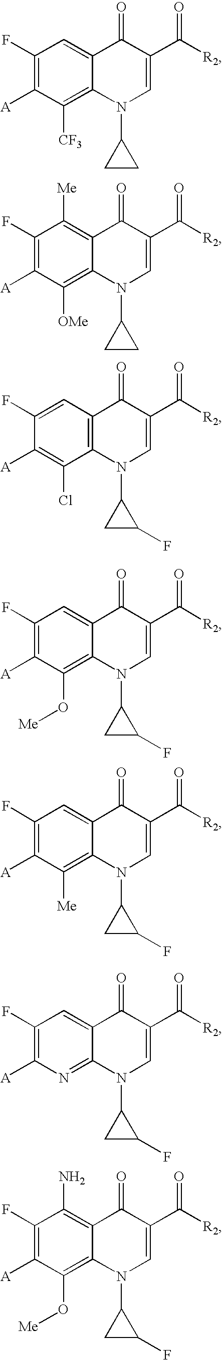 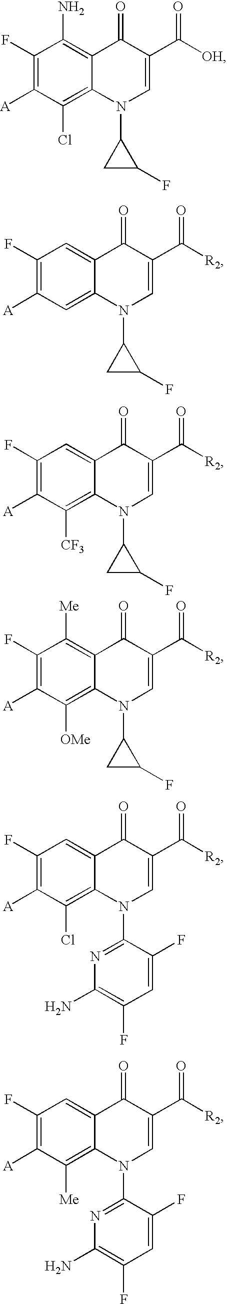 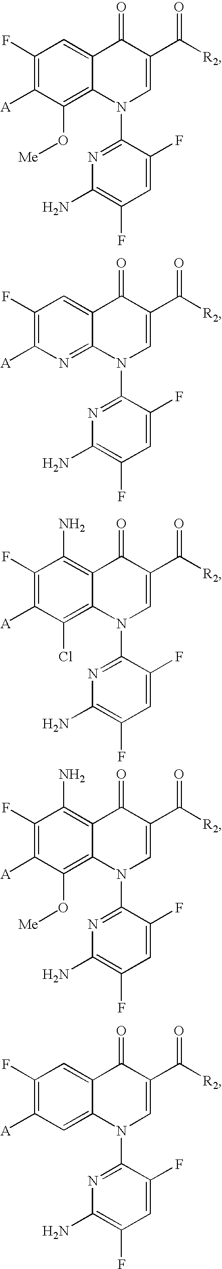 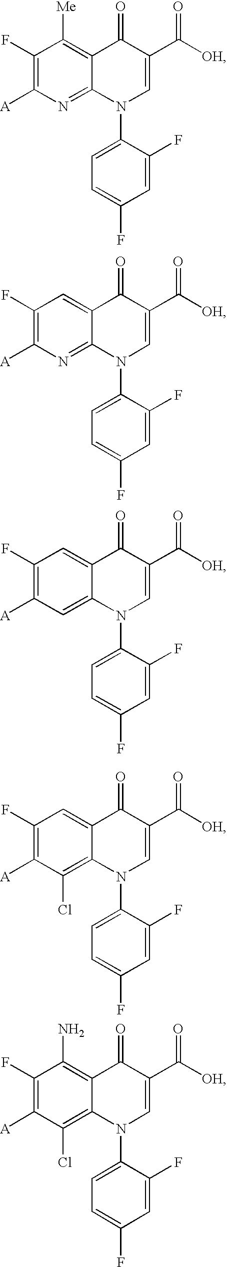 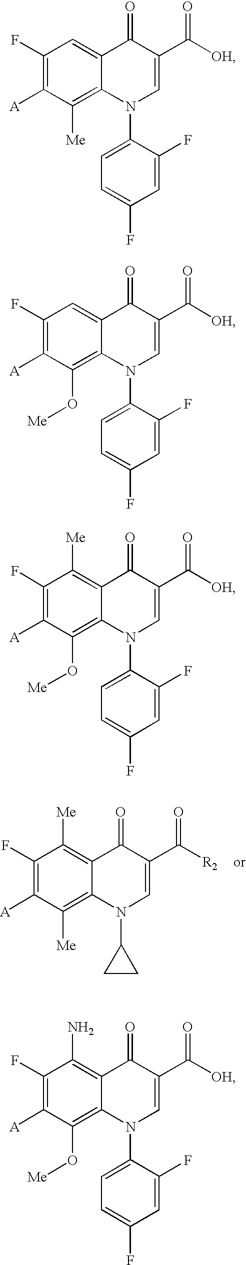
wherein when the compound has formula II, R₂ is OH, O(C₁-C₆)alkyl, or OBF₂,
R₄ is H or F and wherein A is 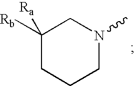
wherein when the compound has formula III, R₂ is OH, O(C₁-C₆)alkyl, or OBF₂,
R₄ is H or F and A is 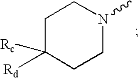
wherein when the compound has formula IV, R₂ is OH, O(C₁-C₆)alkyl, or OBF₂,
R₄ is H or F, and A is 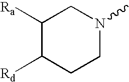
wherein when the compound has formula V, R₂ is OH, O(C₁-C₆)alkyl, or OBF₂,
R₄ is H or F, and A is

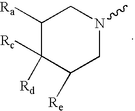

11. The compound of claim 10, wherein 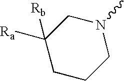
is 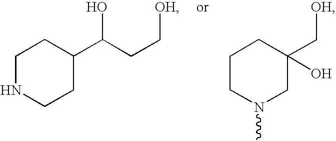
wherein “ 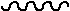
” indicates the point of attachment.

12. A compound of claim 1 which is 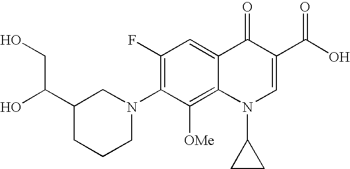
1-Cyclopropyl-7-[3-(1,2-dihydroxy-ethyl)-piperidin-1-yl]-6-fluoro-8-methoxy-4-oxo-1,4-dihydro-quinoline-3-carboxylic acid; 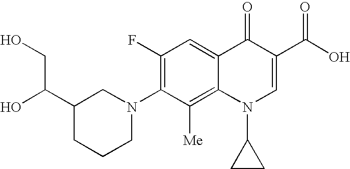
1-Cyclopropyl-7-[3-(1,2-dihydroxy-ethyl)-piperidin-1-yl]-6-fluoro-8-methyl-4-oxo-1,4-dihydro-quinoline-3-carboxylic acid; 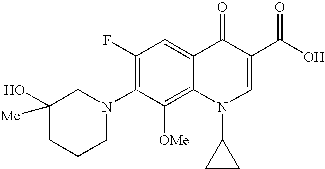
1-Cyclopropyl-6-fluoro-7-(3-hydroxy-3-methyl-piperidin-1-yl)-8-methoxy-4-oxo-1,4-dihydro-quinoline-3-carboxylic acid; 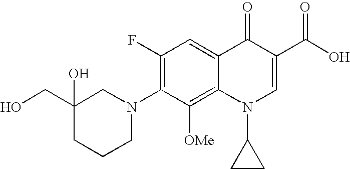
1-Cyclopropyl-6-fluoro-7-(3-hydroxy-3-hydroxymethyl-piperidin-1-yl)-8-methoxy-4-oxo-1,4-dihydro-quinoline-3-carboxylic acid; 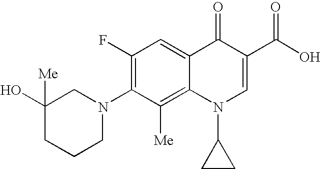
1-Cyclopropyl-6-fluoro-7-(3-hydroxy-3-methyl-piperidin-1-yl)-8-methyl-4-oxo-1,4-dihydro-quinoline-3-carboxylic acid;
1-Cyclopropyl-7-[3-(1,2-dihydroxy-ethyl)-piperidin-1-yl]-8-methoxy-4-oxo-1,4-dihydro-quinoline-3-carboxylic acid;
1-Cyclopropyl-7-[3-(1,2-dihydroxy-ethyl)-piperidin-1-yl]-8-methyl-4-oxo-1,4-dihydro-quinoline-3-carboxylic acid;
1-Cyclopropyl-7-(3-hydroxy-3-methyl-piperidin-1-yl)-8-methoxy-4-oxo-1,4-dihydro-quinoline-3-carboxylic acid;
1-Cyclopropyl-7-(3-hydroxy-3-hydroxymethyl-piperidin-1-yl)-8-methoxy-4-oxo-1,4-dihydro-quinoline-3-carboxylic acid;
1-Cyclopropyl-7-(3-hydroxy-3-methyl-piperidin-1-yl)-8-methyl-4-oxo-1,4-dihydro-quinoline-3-carboxylic acid; 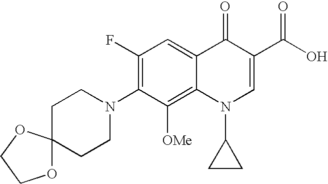
1-Cyclopropyl-7-(1,4-dioxa-8-aza-spiro[4.5]dec-8-yl)-6-fluoro-8-methoxy-4-oxo-1,4-dihydro-quinoline-3-carboxylic acid, 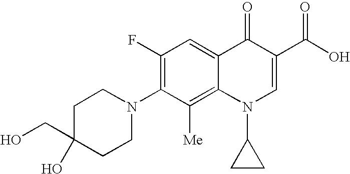
1-Cyclopropyl-6-fluoro-7-(4-hydroxy-4-hydroxymethyl-piperidin-1-yl)-8-methyl-4-oxo-1,4-dihydro-quinoline-3-carboxylic acid; 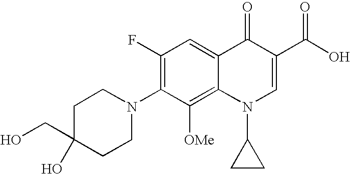
1-Cyclopropyl-6-fluoro-7-(4-hydroxy-4-hydroxymethyl-piperidin-1-yl)-8-methoxy-4-oxo-1,4-dihydro-quinoline-3-carboxylic acid; 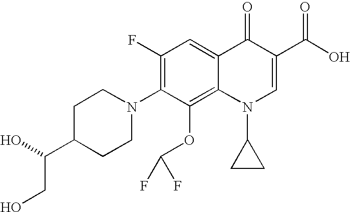
1-Cyclopropyl-8-difluoromethoxy-7-[4-(1,2-dihydroxy-ethyl)-piperidin-1-yl]-6-fluoro-4-oxo-1,4-dihydro-quinoline-3-carboxylic acid; 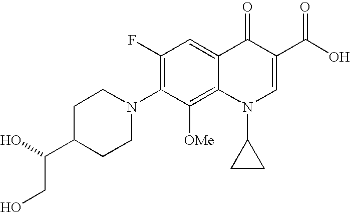
1-Cyclopropyl-8-methoxy-7-[4-(1,2-dihydroxy-ethyl)-piperidin-1-yl]-6-fluoro-4-oxo-1,4-dihydro-quinoline-3-carboxylic acid; 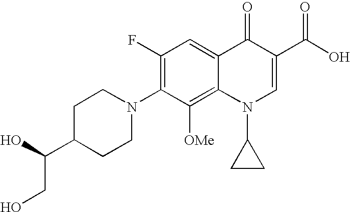
1-Cyclopropyl-8-methyl-7-[4-(1,2-dihydroxy-ethyl)-piperidin-1-yl]-6-fluoro-4-oxo-1,4-dihydro-quinoline-3-carboxylic acid; 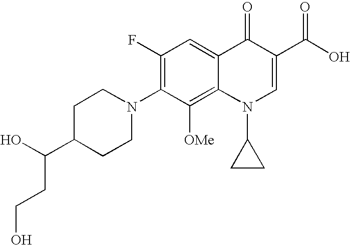
1-Cyclopropyl-7-[4-(1,3-dihydroxy-propyl)-piperidin-1-yl]-6-fluoro-8-methoxy-4-oxo-1,4-dihydro-quinoline-3-carboxylic acid; 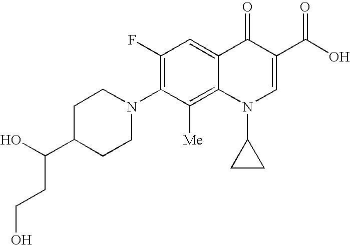
1-Cyclopropyl-7-[4-(1,3-dihydroxy-propyl)-piperidin-1-yl]-6-fluoro-8-methyl-4-oxo-1,4-dihydro-quinoline-3-carboxylic acid; 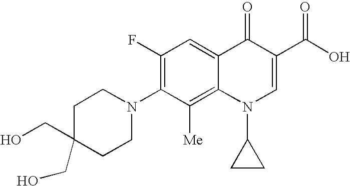
7-(4,4-Bis-hydroxymethyl-piperidin-1-yl)-1-cyclopropyl-6-fluoro-8-methyl-4-oxo-1,4-dihydro-quinoline-3-carboxylic acid; 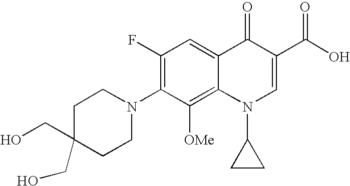
7-(4,4-Bis-hydroxymethyl-piperidin-1-yl)-1-cyclopropyl-6-fluoro-8-methoxy-4-oxo-1,4-dihydro-quinoline-3-carboxylic acid;
1-Cyclopropyl-7-(1,4-dioxa-8-aza-spiro[4.5]dec-8-yl)-8-methoxy-4-oxo-1,4-dihydro-quinoline-3-carboxylic acid; 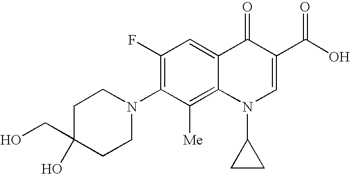
1-Cyclopropyl-7-(4-hydroxy-4-hydroxymethyl-piperidin-1-yl)-8-methyl-4-oxo-1,4-dihydro-quinoline-3-carboxylic acid;
1-Cyclopropyl-7-(4-hydroxy-4-hydroxymethyl-piperidin-1-yl)-8-methoxy-4-oxo-1,4-dihydro-quinoline-3-carboxylic acid;
1-Cyclopropyl-8-difluoromethoxy-7-[4-(1,2-dihydroxy-ethyl)-piperidin-1-yl]-4-oxo-1,4-dihydro-quinoline-3-carboxylic acid;
1-Cyclopropyl-8-methoxy-7-[4-(1,2-dihydroxy-ethyl)-piperidin-1-yl]-4-oxo-1,4-dihydro-quinoline-3-carboxylic acid;
1-Cyclopropyl-8-methyl-7-[4-(1,2-dihydroxy-ethyl)-piperidin-1-yl]-4-oxo-1,4-dihydro-quinoline-3-carboxylic acid;
1-Cyclopropyl-7-[4-(1,3-dihydroxy-propyl)-piperidin-1-yl]-8-methoxy-4-oxo-1,4-dihydro-quinoline-3-carboxylic acid;
1-Cyclopropyl-7-[4-(1,3-dihydroxy-propyl)-piperidin-1-yl]-8-methyl-4-oxo-1,4-dihydro-quinoline-3-carboxylic acid;
7-(4,4-Bis-hydroxymethyl-piperidin-1-yl)-1-cyclopropyl-8-methyl-4-oxo-1,4-dihydro-quinoline-3-carboxylic acid;
7-(4,4-Bis-hydroxymethyl-piperidin-1-yl)-1-cyclopropyl-8-methoxy-4-oxo-1,4-dihydro-guinoline-3-carboxylic acid; 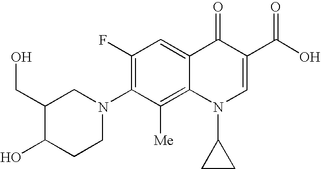
1-Cyclopropyl-6-fluoro-7-(4-hydroxy-3-hydroxymethyl-piperidin-1-yl)-8-methyl-4-oxo-1,4-dihydro-guinoline-3-carboxylic acid; 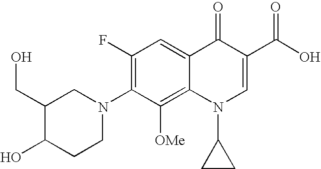
1-Cyclopropyl-6-fluoro-7-(4-hydroxy-3-hydroxymethyl-piperidin-1-yl)-8-methoxy-4-oxo-1,4-dihydro-quinoline-3-carboxylic acid; 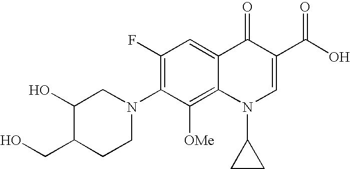
1-Cyclopropyl-6-fluoro-7-(3-hydroxy-4-hydroxymethyl-piperidin-1-yl)-8-methoxy-4-oxo-1,4-dihydro-quinoline-3-carboxylic acid; 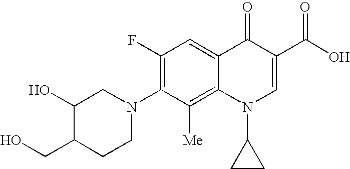
1-Cyclopropyl-6-fluoro-7-(3-hydroxy-4-hydroxymethyl-piperidin-1-yl)-8-methyl-4-oxo-1,4-dihydro-quinoline-3-carboxylic acid; 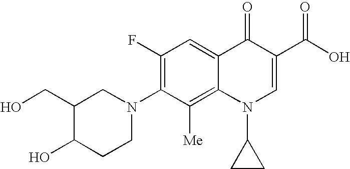
1-Cyclopropyl-6-fluoro-7-(4-hydroxy-3-hydroxymethyl-piperidin-1-yl)-8-methyl-4-oxo-1,4-dihydro-quinoline-3-carboxylic acid; 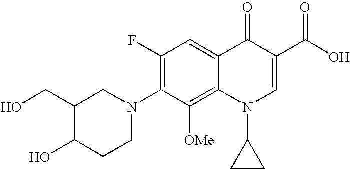
1-Cyclopropyl-6-fluoro-7-(4-hydroxy-3-hydroxymethyl-piperidin-1-yl)-8-methoxy-4-oxo-1,4-dihydro-quinoline-3-carboxylic acid;
1-Cyclopropyl-7-(4-hydroxy-3-hydroxymethyl-piperidin-1-yl)-8-methyl-4-oxo-1,4-dihydro-guinoline-3-carboxylic acid;
1-Cyclopropyl-7-(4-hydroxy-3-hydroxymethyl-piperidin-1-yl)-8-methoxy-4-oxo-1,4-dihydro-guinoline-3-carboxylic acid;
1-Cyclopropyl-7-(3-hydroxy-4-hydroxymethyl-piperidin-1-yl)-8-methoxy-4-oxo-1,4-dihydro-quinoline-3-carboxylic acid;
1-Cyclopropyl-7-(3-hydroxy-4-hydroxymethyl-piperidin-1-yl)-8-methyl-4-oxo-1,4-dihydro-quinoline-3-carboxylic acid;
1-Cyclopropyl-7-(4-hydroxy-3-hydroxymethyl-piperidin-1-yl)-8-methyl-4-oxo-1,4-dihydro-quinoline-3-carboxylic acid; or
1-Cyclopropyl-7-(4-hydroxy-3-hydroxymethyl-piperidin-1-yl)-8-methoxy-4-oxo-1,4-dihydro-guinoline-3-carboxylic acid 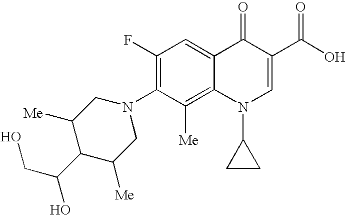
1-Cyclopropyl-7-[4-(1,2-dihydroxy-ethyl)-3,5-dimethyl-piperidin-1-yl]-6-fluoro-8-methyl-4-oxo-1,4-dihydro-guinoline-3-carboxylic acid 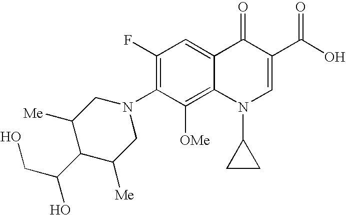
1-Cyclopropyl-7-[4-(1,2-dihydroxy-ethyl)-3,5-dimethyl-piperidin-1-yl]-6-fluoro-8-methoxy-4-oxo-1,4-dihydro-guinoline-3-carboxylic acid; 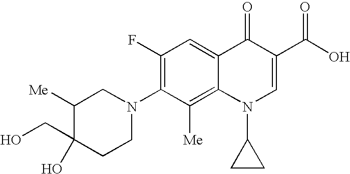
1-Cyclopropyl-6-fluoro-7-(4-hydroxy-4-hydroxymethyl-3-methyl-piperidin-1-yl)-8-methyl-4-oxo-1,4-dihydro-quinoline-3-carboxylic acid; 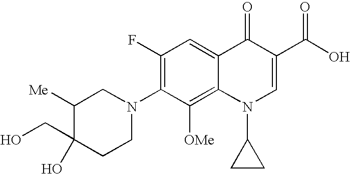
1-Cyclopropyl-6-fluoro-7-(4-hydroxy-4-hydroxymethyl-3-methyl-piperidin-1-yl)-8-methoxy-4-oxo-1,4-dihydro-quinoline-3-carboxylic acid; 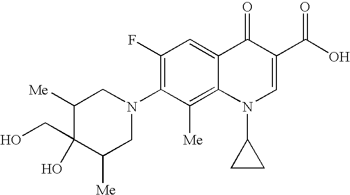
1-Cyclopropyl-6-fluoro-7-(4-hydroxy-4-hydroxymethyl-3,5-dimethyl-piperidin-1-yl)-8-methyl-4-oxo-1,4-dihydro-quinoline-3-carboxylic acid; 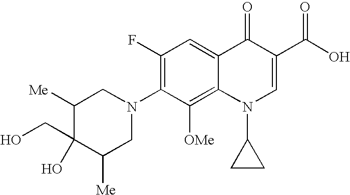
1-Cyclopropyl-6-fluoro-7-(4-hydroxy-4-hydroxymethyl-3,5-dimethyl-piperidin-1-yl)-8-methoxy-4-oxo-1,4-dihydro-quinoline-3-carboxylic acid; 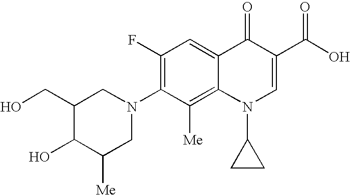
1-Cyclopropyl-6-fluoro-7-(4-hydroxy-3-hydroxymethyl-5-methyl-piperidin-1-yl)-8-methyl-4-oxo-1,4-dihydro-quinoline-3-carboxylic acid; 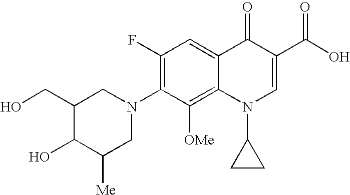
1-Cyclopropyl-6-fluoro-7-(4-hydroxy-3-hydroxymethyl-5-methyl-piperidin-1-yl)-8-methoxy-4-oxo-1,4-dihydro-quinoline-3-carboxylic acid; 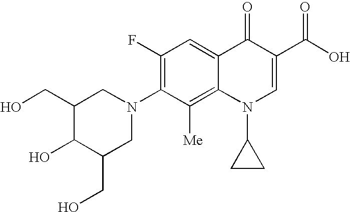
1-Cyclopropyl-6-fluoro-7-(4-hydroxy-3,5-bis-hydroxymethyl-piperidin-1-yl)-8-methyl-4-oxo-1,4-dihydro-quinoline-3-carboxylic acid; 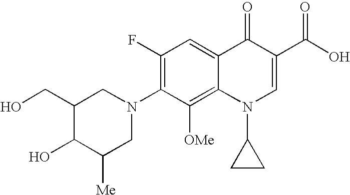
1-Cyclopropyl-6-fluoro-7-(4-hydroxy-3,5-bis-hydroxymethyl-piperidin-1-yl)-8-methoxy-4-oxo-1,4-dihydro-quinoline-3-carboxylic acid;
1-Cyclopropyl-7-[4-(1,2-dihydroxy-ethyl)-3,5-dimethyl-piperidin-1-yl]-8-methyl-4-oxo-1,4-dihydro-quinoline-3-carboxylic acid,
1-Cyclopropyl-7-[4-(1,2-dihydroxy-ethyl)-3,5-dimethyl-piperidin-1-yl]-8-methoxy-4-oxo-1,4-dihydro-quinoline-3-carboxylic acid;
1-Cyclopropyl-7-(4-hydroxy-4-hydroxymethyl-3-methyl-piperidin-1-yl)-8-methyl-4-oxo-1,4-dihydro-quinoline-3-carboxylic acid;
1-Cyclopropyl-7-(4-hydroxy-4-hydroxymethyl-3-methyl-piperidin-1-yl)-8-methoxy-4-oxo-1,4-dihydro-guinoline-3-carboxylic acid;
1-Cyclopropyl-7-(4-hydroxy-4-hydroxymethyl-3,5-dimethyl-piperidin-1-yl)-8-methyl-4-oxo-1,4-dihydro-guinoline-3-carboxylic acid;
1-Cyclopropyl-7-(4-hydroxy-4-hydroxymethyl-3,5-dimethyl-piperidin-1-yl)-8-methoxy-4-oxo-1,4-dihydro-guinoline-3-carboxylic acid;
1-Cyclopropyl-7-(4-hydroxy-3-hydroxymethyl-5-methyl-piperidin-1-yl)-8-methyl-4-oxo-1,4-dihydro-quinoline-3-carboxylic acid;
1-Cyclopropyl-7-(4-hydroxy-3-hydroxymethyl-5-methyl-piperidin-1-yl)-8-methoxy-4-oxo-1,4-dihydro-quinoline-3-carboxylic acid;
1-Cyclopropyl-7-(4-hydroxy-3,5-bis-hydroxymethyl-piperidin-1-yl)-8-methyl-4-oxo-1,4-dihydro-quinoline-3-carboxylic acid; or
1-Cyclopropyl-7-(4-hydroxy-3,5-bis-hydroxymethyl-piperidin-1-yl)-8-methoxy-4-oxo-1,4-dihydro-quinoline-3-carboxylic acid 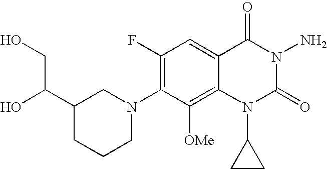
3-Amino-1-cyclopropyl-7-[3-(1,2-dihydroxy-ethyl)-piperidin-1-yl]-6-fluoro-8-methoxy 1H-quinazoline-2,4-dione; 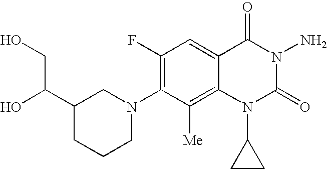
3-Amino-1-cyclopropyl-7-[3-(1,2-dihydroxy-ethyl)-piperidin-1-yl]-6-fluoro-8-methyl-1H-quinazoline-2,4-dione; 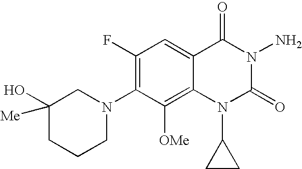
3-Amino-1-cyclopropyl-6-fluoro-7-(3-hydroxy-3-methyl-piperidin-1-yl)-8-methoxy-1H-quinazoline-2,4-dione; 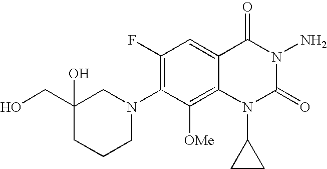
3-Amino-1-cyclopropyl-6-fluoro-7-(3-hydroxy-3-hydroxymethyl-piperidin-1-yl)-8-methoxy-1H-quinazoline-2,4-dione; 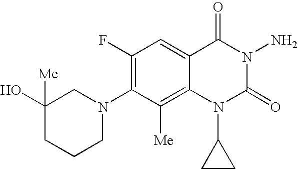
3-Amino-1-cyclopropyl-6-fluoro-7-(3-hydroxy-3-methyl-piperidin-1-yl)-8-methyl-1H-quinazoline-2,4-dione; 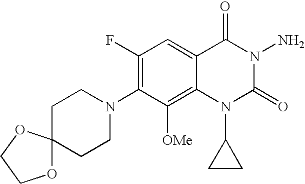
3-Amino-1-cyclopropyl-7-(1,4-dioxa-8-aza-spiro[4.5]dec-8-yl)-6-fluoro-8-methoxy-1H-quinazoline-2,4-dione; 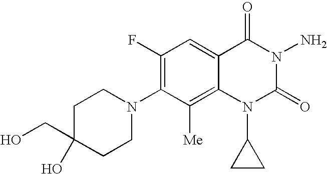
3-Amino-1-cyclopropyl-6-fluoro-7-(4-hydroxy-4-hydroxymethyl-piperidin-1-yl)-8-methyl-1H-quinazoline-2,4-dione; 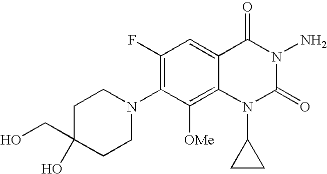
3-Amino-1-cyclopropyl-6-fluoro-7-(4-hydroxy-4-hydroxymethyl-piperidin-1-yl)-8-methoxy-1H-quinazoline-2,4-dione; 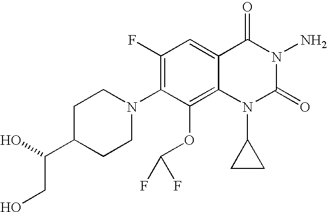
3-Amino-1-cyclopropyl-8-difluoromethoxy-7-[4-(1,2-dihydroxy-ethyl)-piperidin-1-yl]-6-fluoro-1H-quinazoline-2,4-dione; 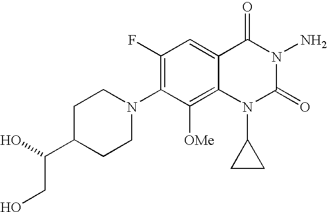
3-Amino-1-cyclopropyl-7-[4-(1,2-dihydroxy-ethyl)-piperidin-1-yl]-6-fluoro-8-methoxy-1H-quinazoline-2,4-dione; 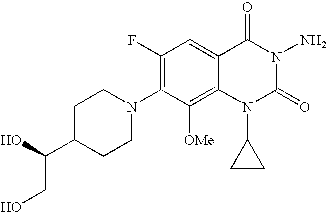
3-Amino-1-cyclopropyl-7-[4-(1,2-dihydroxy-ethyl)-piperidin-1-yl]-6-fluoro-8-methoxy-1H-quinazoline-2,4-dione; 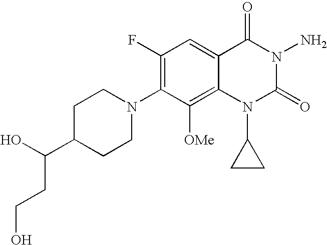
3-Amino-1-cyclopropyl-7-[4-(1,3-dihydroxy-propyl)-piperidin-1-yl-6-fluoro-8-methoxy-1H-quinazoline-2,4-dione, 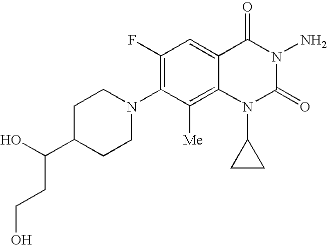
3-Amino-1-cyclopropyl-7-[4-(1,3-dihydroxy-propyl)-piperidin-1-yl]-6-fluoro-8-methyl-1H-quinazoline-2,4-dione; 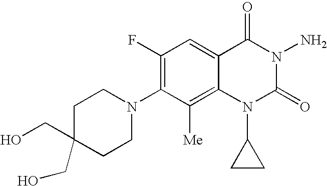
3-Amino-7-(4,4-bis-hydroxymethyl-piperidin-1-yl)-1-cyclopropyl-6-fluoro-8-methyl-1H-quinazoline-2,4-dione; 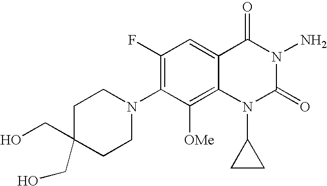
3-Amino-7-(4,4-bis-hydroxymethyl-piperidin-1-yl)-1-cyclopropyl-6-fluoro-8-methoxy-1H-quinazoline-2,4-dione; 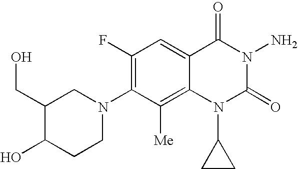
3-Amino-1-cyclopropyl-6-fluoro-7-(4-hydroxy-3-hydroxymethyl-piperidin-1-yl)-8-methyl-1H-quinazoline-2,4-dione; 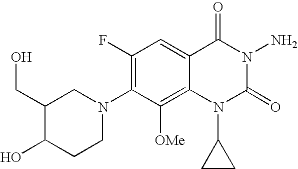
3-Amino-1-cyclopropyl-6-fluoro-7-(4-hydroxy-3-hydroxymethyl-piperidin-1-yl)-8-methoxy-1H-quinazoline-2,4-dione; 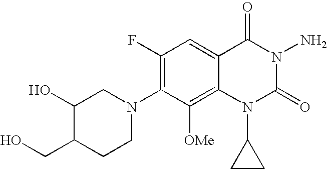
3-Amino-1-cyclopropyl-6-fluoro-7-(3-hydroxy-4-hydroxymethyl-piperidin-1-yl)-8-methoxy-1H-quinazoline-2,4-dione; 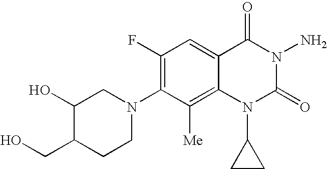
3-Amino-1-cyclopropyl-6-fluoro-7-(3-hydroxy-4-hydroxymethyl-piperidin-1-yl)-8-methyl-1H-quinazoline-2,4-dione; 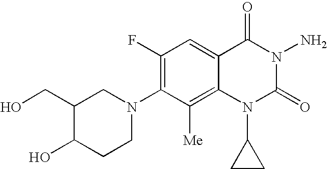
3-Amino-1-cyclopropyl-6-fluoro-7-(4-hydroxy-3-hydroxymethyl-piperidin-1-yl)-8-methyl-1H-quinazoline-2,4-dione, 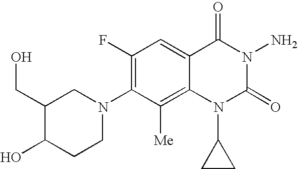
3-Amino-1-cyclopropyl-6-fluoro-7-(4-hydroxy-3-hydroxymethyl-piperidin-1-yl)-8-methoxy-1H-quinazoline-2,4-dione; 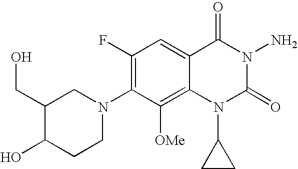
3-Amino-1-cyclopropyl-6-fluoro-7-(4-hydroxy-3-hydroxymethyl-piperidin-1-yl)-8-methoxy-1H-quinazoline-2,4-dione; 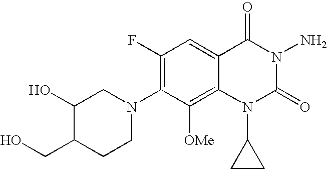
3-Amino-1-cyclopropyl-6-fluoro-6-7-(3-hydroxy-4-hydroxymethyl-piperidin-1-yl)-8-methoxy-1H-quinazoline-2,4-dione, 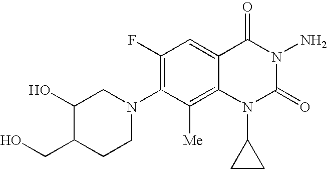
3-Amino-1-cyclopropyl-6-fluoro-7-(3-hydroxy-4-hydroxymethyl-piperidin-1-yl)-8-methyl-1H-quinazoline-2,4-dione; 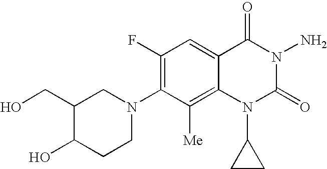
3-Amino-1-cyclopropyl-6-fluoro-7-(3-hydroxy-4-hydroxymethyl-piperidin-1-yl)-8-methyl-1H-quinazoline-2,4-dione,
3-Amino-1-cyclopropyl-7-[3-(1,2-dihydroxy-ethyl)-piperidin-1-yl]-8-methoxy-1H-quinazoline-2,4-dione,
3-Amino-1-cyclopropyl-7-[3-(1,2-dihydroxy-ethyl)-piperidin-1-yl]-8-methyl-1H-quinazoline-2,4-dione;
3-Amino-1-cyclopropyl-7-(3-hydroxy-3-meth yl-piperidin-1-yl)-8-methoxy-1H-quinazoline-2,4-dione;
3-Amino-1-cyclopropyl-7-(3-hydroxy-3-hydroxymethyl-piperidin-1-yl)-8-methoxy-1H-quinazoline-2,4-dione;
3-Amino-1-cyclopropyl-7-(3-hydroxy-3-methyl-piperidin-1-yl)-8-methyl-1H-quinazoline-2,4-dione;
3-Amino-1-cyclopropyl-7-(1,4-dioxa-8-aza-spiro[4.5]dec-8-yl)-8-methoxy-1H-quinazoline-2,4-dione;
3-Amino-1-cyclopropyl-7-(4-hydroxy-4-hydroxymethyl-piperidin-1-yl)-8-methyl-1H-quinazoline-2,4-dione;
3-Amino-1-cyclopropyl-7-(4-hydroxy-4-hydroxymethyl-piperidin-1-yl)-8-methoxy-1H-quinazoline-2,4-dione;
3-Amino-1-cyclopropyl-8-difluoromethoxy-7-[4-(1,2-dihydroxy-ethyl)-piperidin-1-yl]-6-fluoro-1H-quinazoline-2,4-dione;
3-Amino-1-cyclopropyl-7-[4-(1,2-dihydroxy-ethyl)-piperidin-1-yl]-8-methoxy-1H-quinazoline-2,4-dione;
3-Amino-1-cyclopropyl-7-[4-(1,2-dihydroxy-ethyl)-piperidin-1-yl]-8-methoxy-1H-quinazoline-2,4-dione;
3-Amino-1-cyclopropyl-7-[4-(1,3-dihydroxy-propyl)-piperidin-1-yl]-8-methoxy-1H-quinazoline-2,4-dione;
3-Amino-1-cyclopropyl-7-[4-(1,3-dihydroxy-propyl)-piperidin-1-yl]-8-methyl-1H-quinazoline-2,4-dione;
3-Amino-7-(4,4-bis-hydroxymethyl-piperidin-1-yl)-1-cyclopropyl-8-methyl-1H-quinazoline-2,4-dione;
3-Amino-7-(4,4-bis-hydroxymethyl-piperidin-1-yl)-1-cyclopropyl-8-methoxy-1H-quinazoline-2,4-dione;
3-Amino-1-cyclopropyl-7-(4-hydroxy-3-hydroxymethyl-piperidin-1-yl)-8-methyl-1H-quinazoline-2,4-dione;
3-Amino-1-cyclopropyl-7-(4-hydroxy-3-hydroxymethyl-piperidin-1-yl)-8-methoxy-1H-quinazoline-2,4-dione;
3-Amino-1-cyclopropyl-7-(3-hydroxy-4-hydroxymethyl-piperidin-1-yl)-8-methoxy-1H-quinazoline-2,4-dione;
3-Amino-1-cyclopropyl-7-(3-hydroxy-4-hydroxymethyl-piperidin-1-yl)-8-methyl-1H-quinazoline-2,4-dione;
3-Amino-1-cyclopropyl-7-(4-hydroxy-3-hydroxymethyl-piperidin-1-yl)-8-methyl-1H-quinazoline-2,4-dione;
3-Amino-1-cyclopropyl-7-(4-hydroxy-3-hydroxymethyl-piperidin-1-yl)-8-methyl-1H-quinazoline-2,4-dione;
3-Amino-1-cyclopropyl-7-(4-hydroxy-3-hydroxymethyl-piperidin-1-yl)-8-methoxy-1H-quinazoline-2,4-dione;
3-Amino-1-cyclopropyl-7-(3-hydroxy-4-hydroxymethyl-piperidin-1-yl)-8-methoxy-1H-quinazoline-2,4-dione;
3-Amino-1-cyclopropyl-7-(3-hydroxy-4-hydroxymethyl-piperidin-1-yl)-8-methyl-1H-quinazoline-2,4-dione;
3-Amino-1-cyclopropyl-7-(3-hydroxy-4-hydroxymethyl-piperidin-1-yl)-8-methyl-1H-quinazoline-2,4-dione;
1-cyclopropyl-7-[3-(1,2-dihydroxy-ethyl)-piperidin-1-yl]-8-methoxy-1H-quinazoline-2,4-dione;
1-cyclopropyl-7-[3-(1,2-dihydroxy-ethyl)-piperidin-1-yl]-8-methyl-1H-quinazoline-2,4-dione;
1-cyclopropyl-7-(3-hydroxy-3-methyl-piperidin-1-yl)-8-methoxy-1H-quinazoline-2,4-dione;
1-cyclopropyl-7-(3-hydroxy-3-hydroxymethyl-piperidin-1-yl]-8-methoxy-1H-quinazoline-2,4-dione;
1-cyclopropyl-7-(3-hydroxy-3-methyl-piperidin-1-yl)-8-methyl-1H-quinazoline-2,4-dione;
1-cyclopropyl-7-(1,4-dioxa-8-aza-spiro[4.5]dec-8-yl)-8-methoxy-1H-quinazoline-2,4-dione; 555555555
1-cyclopropyl-7-(4-hydroxy-4-hydroxymethyl-piperidin-1-yl)-8-methyl-1H-quinazoline-2,4-dione;
1-cyclopropyl-7-(4-hydroxy-4-hydroxymethyl-piperidin-1-yl)-8-methoxy-1H-quinazoline-2,4-dione;
1-cyclopropyl-8-difluoromethoxy-7-[4-(1,2-dihydroxy-ethyl)-piperidin-1-yl]-6-fluoro-1H-quinazoline-2,4-dione;
1-cyclopropyl-7-[4-(1,2-dihydroxy-ethyl)-piperidin-1-yl]-8-methoxy-1H-quinazoline-2,4-dione;
1-cyclopropyl-7-[4-(1,2-dihydroxy-ethyl)-piperidin-1-yl]-8-methoxy-1H-quinazoline-2,4-dione;
1-cyclopropyl-7-[4-(1,3-dihydroxy-propyl)-piperidin-1-yl]-8-methoxy-1H-quinazoline-2,4-dione;
1-cyclopropyl-7-[4-(1,3-dihydroxy-propyl)-piperidin-1-yl]-8-methyl-1H-quinazoline-2,4-dione;
7-(4,4-bis-hydroxymethyl-piperidin-1-yl)-1-cyclopropyl-8-methyl-1H-quinazoline-2,4-dione;
7-(4,4-bis-hydroxymethyl-piperidin-1-yl)-1-cyclopropyl-8-methoxy-1H-quinazoline-2,4-dione;
1-cyclopropyl-7-(4-hydroxy-3-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methyl-1H-quinazoline-2,4-dione;
1-cyclopropyl-7-(4-hydroxy-3-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methoxy-1H-quinazoline-2,4-dione;
1-cyclopropyl-7-(3-hydroxy-4-hydroxymethyl-piperidin-1-yl)-8-methyl-1H-quinazoline-2,4-dione;
1-cyclopropyl-7-(3-hydroxy-4-hydroxymethyl-piperidin-1-yl)-8-methyl-1H-quinazoline-2,4-dione;
1-cyclopropyl-7-[3-(1,2-dihydroxy-ethyl)-piperidin-1-yl]-6-fluoro-8-methoxy-1H-quinazoline-2,4-dione;
1-cyclopropyl-7-[3-(1,2-dihydroxy-ethyl)-piperidin-1-yl]-6-fluoro-8-methyl-1H-quinazoline-2,4-dione;
1-cyclopropyl-6-fluoro-7-(3-hydroxy-3-methyl-piperidin-1-yl)-8-methoxy-1H-quinazoline-2,4-dione;
1-cyclopropyl-6-fluoro-7-(3-hydroxy-3-hydroxymethyl-piperidin-1-yl)-8-methoxy-1H-quinazoline-2,4-dione;
1-cyclopropyl-6-fluoro-7-(3-hydroxy-3-methyl-piperidin-1-yl)-8-methyl-1H-quinazoline-2,4-dione;
1-cyclopropyl-7-(1,4-dioxa-8-aza-spiro[4.5]dec-8-yl)-6-fluoro-8-methoxy-1H-quinazoline-2,4-dione;
1-cyclopropyl-6-fluoro-7-(4-hydroxy-4-hydroxymethyl-piperidin-1-yl)-8-methyl-1H-quinazoline-2,4-dione;
1-cyclopropyl-6-fluoro-7-(4-hydroxy-4-hydroxymethyl-piperidin-1-yl)-8-methoxy-1H-quinazoline-2,4-dione;
1-cyclopropyl-8-difluoromethoxy-7-[4-(1,2-dihydroxy-ethyl)-piperidin-1-yl]-6-fluoro-1H-quinazoline-2,4-dione;
1-cyclopropyl-7-[4-(1,2-dihydroxy-ethyl)-piperidin-1-yl]-6-fluoro-8-methoxy-1H-quinazoline-2,4-dione;
1-cyclopropyl-7-[4-(1,2-dihydroxy-ethyl)-piperidin-1-yl]-6-fluoro-8-methoxy-1H-quinazoline-2,4-dione;
1-cyclopropyl-7-[4-(1,3-dihydroxy-propyl)-piperidin-1-yl]-6-fluoro-8-methoxy-1H-quinazoline-2,4-dione;
1-cyclopropyl-7-[4-(1,3-dihydroxy-propyl)-piperidin-1-yl]-6-fluoro-8-methyl-1H-quinazoline-2,4-dione;
7-(4,4-bis-hydroxymethyl-piperidin-1-yl)-1-cyclopropyl-6-fluoro-8-methyl-1H-quinazoline-2,4-dione;
7-(4,4-bis-hydroxymethyl-piperidin-1-yl)-1-cyclopropyl-6-fluoro-8-methoxy-1H-quinazoline-2,4-dione;
1-cyclopropyl-6-fluoro-7-(4-hydroxy-3-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methyl-1H-quinazoline-2,4-dione;
1-cyclopropyl-6-fluoro-7-(4-hydroxy-3-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methoxy-1H-quinazoline-2,4-dione;
1-cyclopropyl-6-fluoro-7-(3-hydroxy-4-hydroxymethyl-piperidin-1-yl)-8-methyl-1H-quinazoline-2,4-dione; or
1-cyclopropyl-6-fluoro-7-(3-hydroxy-4-hydroxymethyl-piperidin-1-yl)-8-methyl-1H-quinazoline-2,4-dione.

13-16. (canceled)

17. The compound of claim 10, wherein 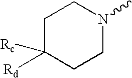
is

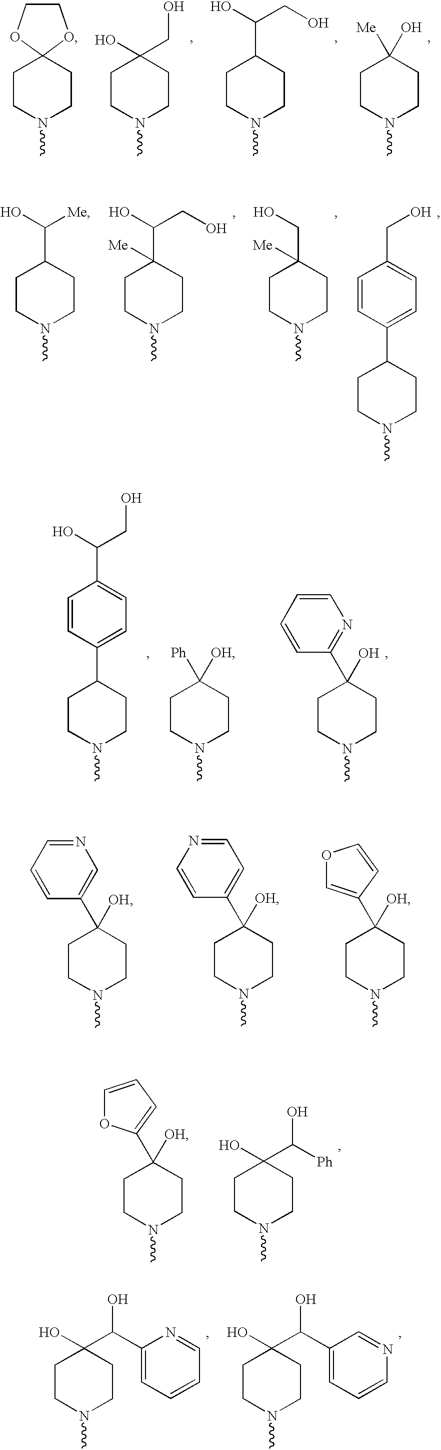

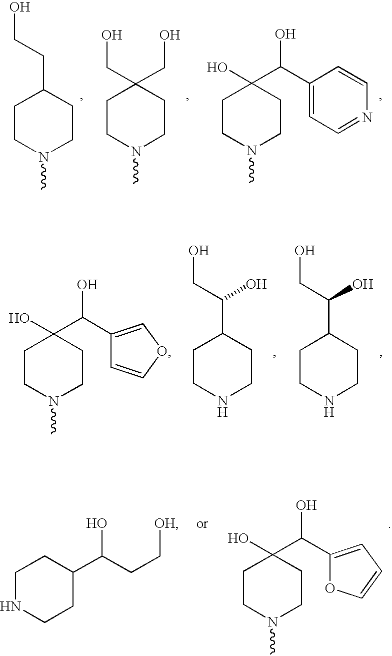

18-22. (canceled)

23. The compound of claim 10, wherein 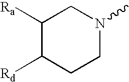
is

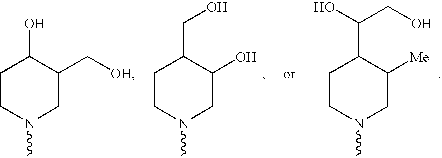

24-28. (canceled)

29. The compound of claim 10, wherein 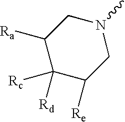
is 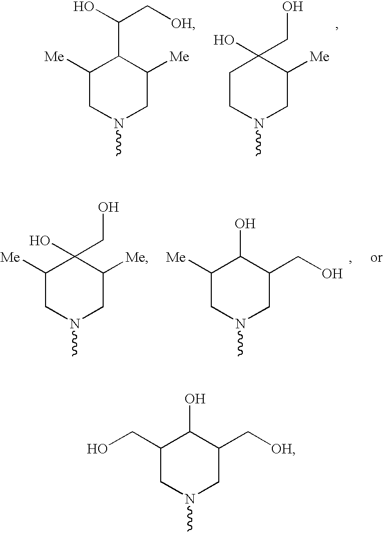
wherein “ 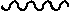
” indicates the point of attachment.

30-33. (canceled)

34. The compound of claim 10 having the structure of formula VI, which is: 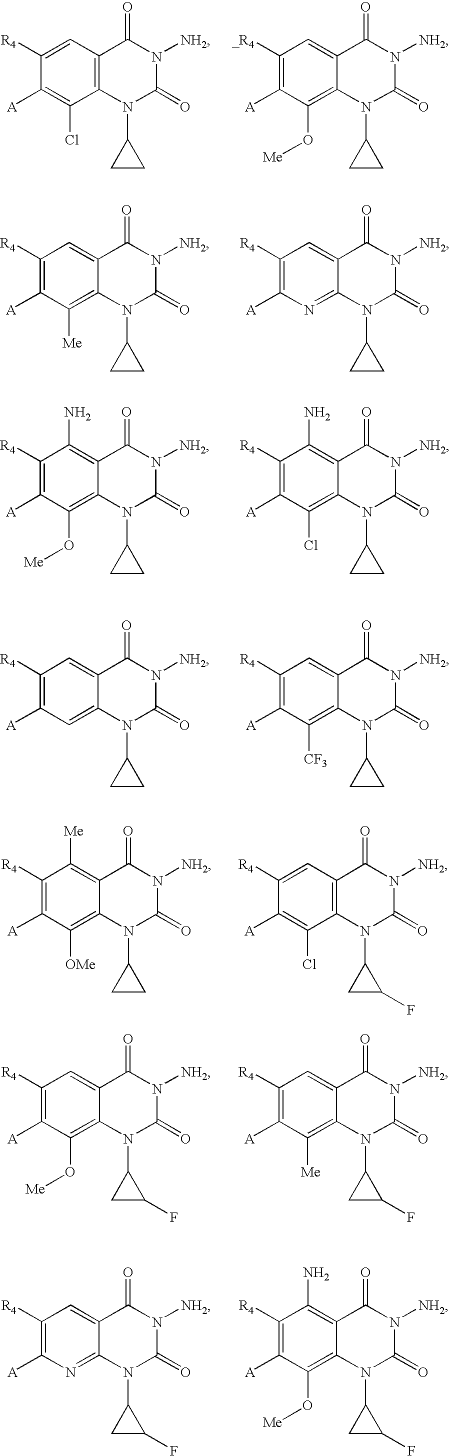 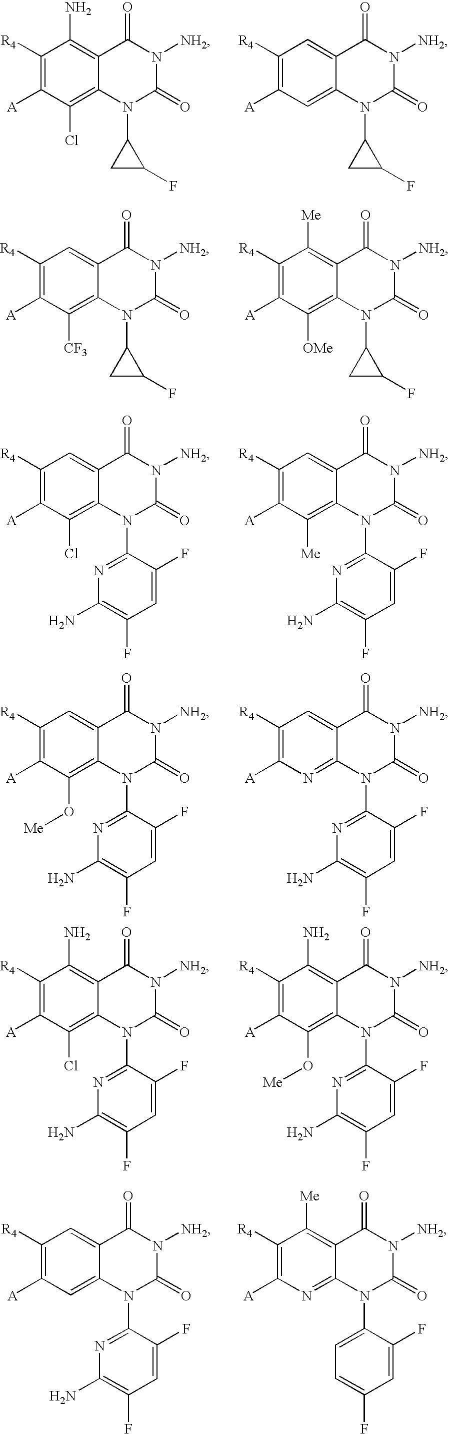 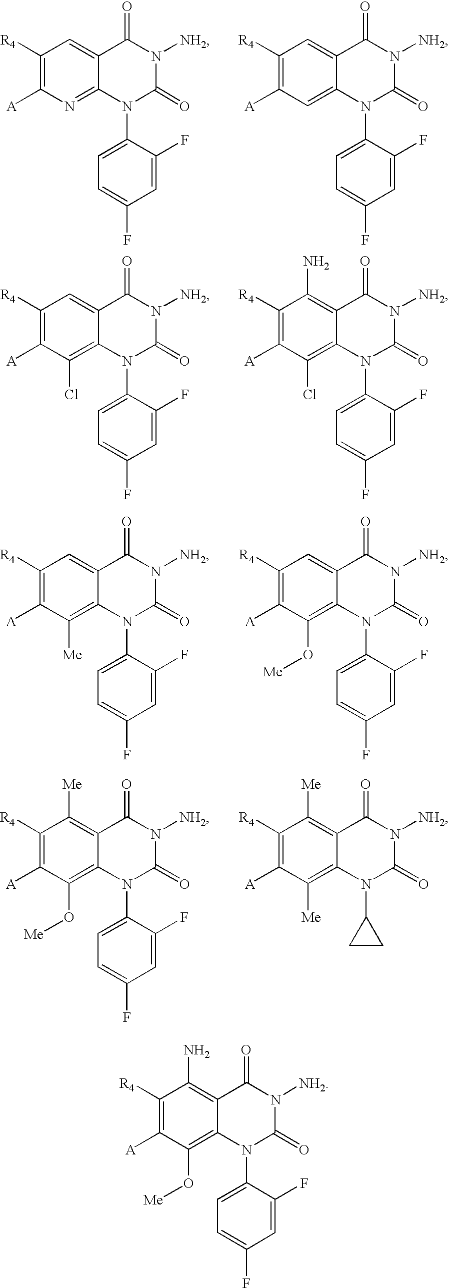 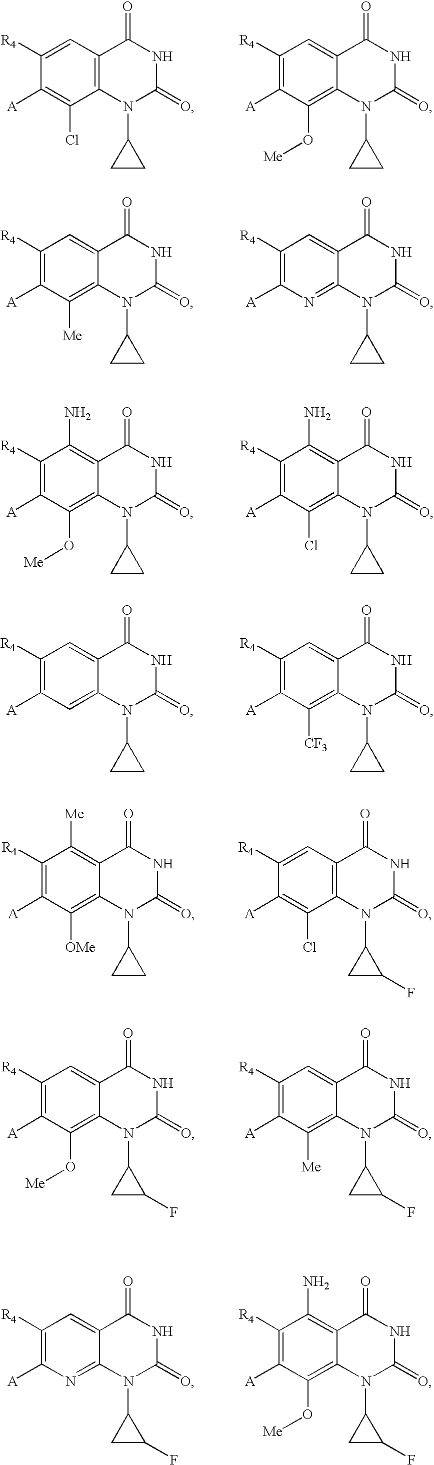 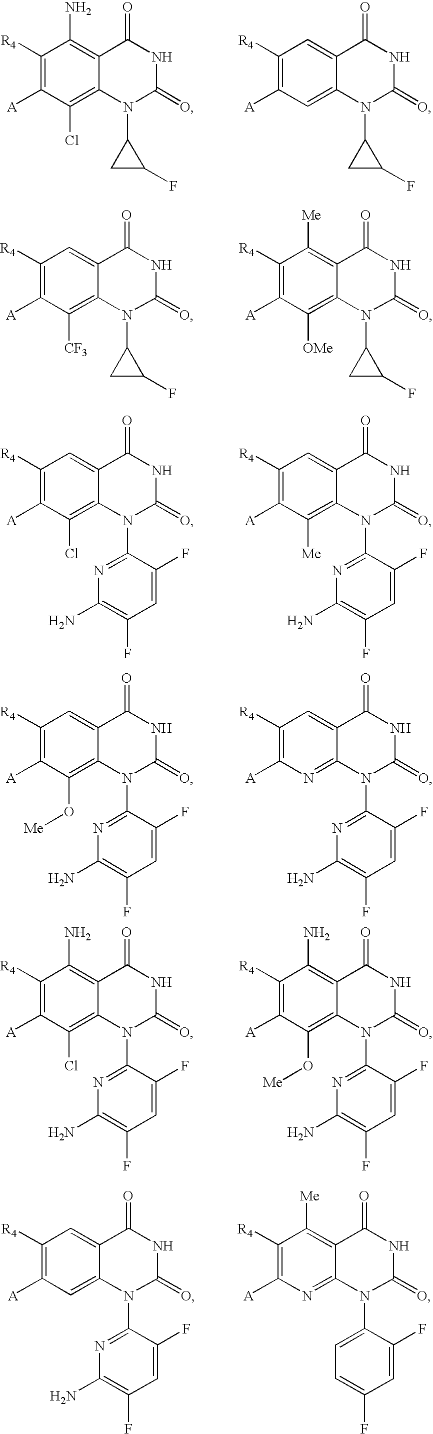 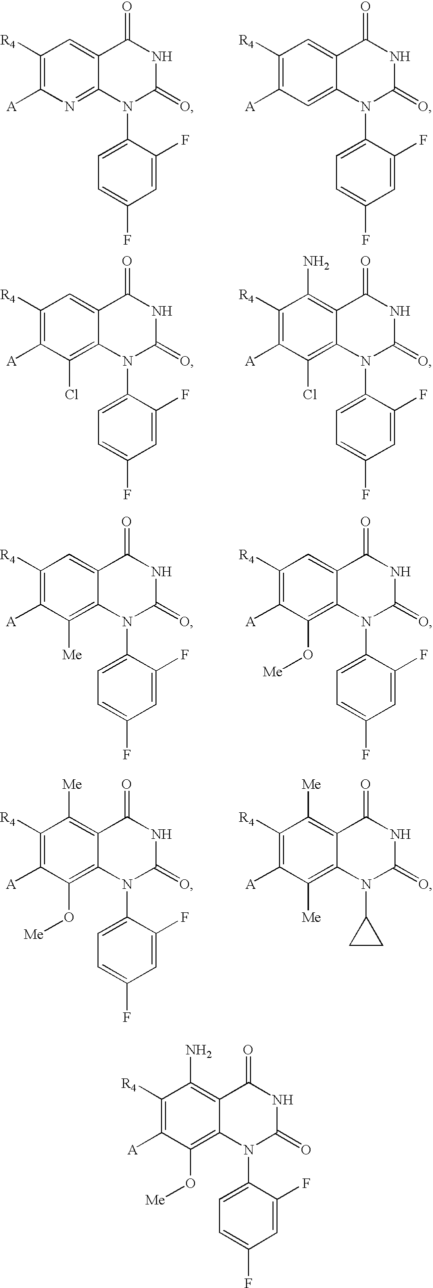
wherein R₄ is H or F, and wherein A is

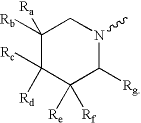

35. The compound of claim 34, wherein R_{g} in wherein A is H.

36. The compound of claim 35, wherein 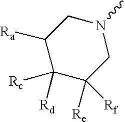
is

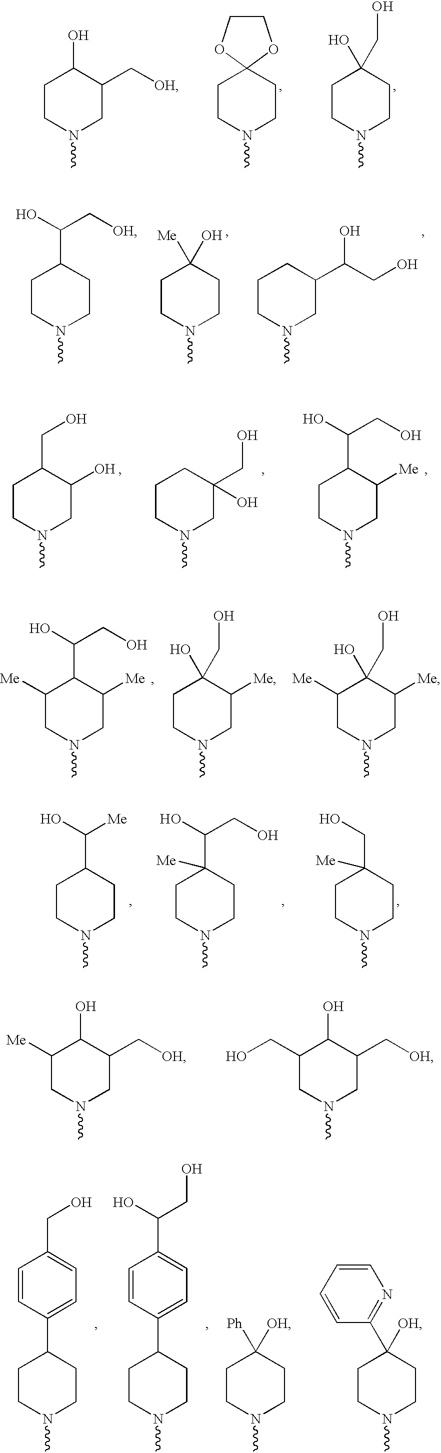

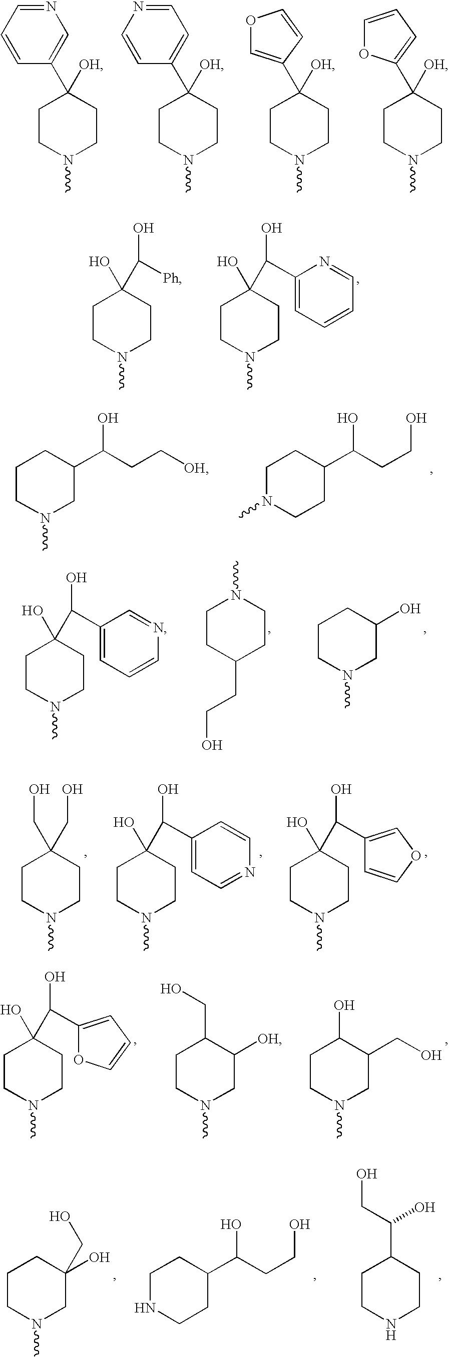

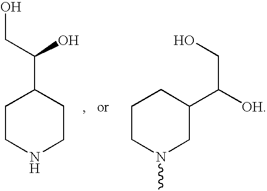

37. (canceled)

38. A pharmaceutical formulation comprising a compound of formula I, II, III, IV, V, or VI admixed with a pharmaceutically acceptable diluent, carrier, or excipient.

39. A method of treating a bacterial infection in a mammal, comprising administering to a mammal in need thereof an effective amount of a compound of a compound of formula I, II, III, IV, V, or VI.
